SEQUENCE LISTING (39 sequences)

SEQ ID NO: 1 - protein, 482 aa, Clitoria ternatea
MKNPLAILFLIATVVAVVSGIRDDFLRLPSQASKFFQADDNVEGTRWAVLVAGSKGYVNYRHQADVCHAYQILKKGGLKDENIIVFMYDDIAYNESNPHPGVIINHPYGSDVYKGVPKDYVGEDINPPNFYAVLLANKSALTGTGSGKVLDSGPNDHVFIYYTDHGGAGVLGMPSKPYIAASDLNDVLKKKHASGTYKSIVFYVESCESGSMFDGLLPEDHNIYVMGASDTGESSWVTYCPLQHPSPPPEYDVCVGDLFSVAWLEDCDVHNLQTETFQQQYEVVKNKTIVALIEDGTHVVQYGDVGLSKQTLFVYMGTDPANDNNTFTDKNSLGTPRKAVSQRDADLIHYWEKYRRAPEGSSRKAEAKKQLREVMAHRMHIDNSVKHIGKLLFGIEKGHKMLNNVRPAGLPVVDDWDCFKTLIRTFETHCGSLSEYGMKHMRSFANLCNAGIRKEQMAEASAQACVSIPDNPWSSLHAGFSV

SEQ ID NO: 2 - protein, 480 aa, Clitoria ternatea
  source 1..480: note = sequence comprises His Tag, G252V and G182A mutations
MGHHHHHHSSGVDLGTENLYFQSMARLNPQKEWDSVIRLPTEPVDADTDEVGTRWAVLVAGSNGYENYRHQADVCHAYQLLIKGGLKEENIVVFMYDDIAWHELNPRPGVIINNPRGEDVYAGVPKDYTGEDVTAENLFAVILGDRSKVKGGSGKVINSKPEDRIFIFYSDHGAPGVLGMPNEQILYAMDFIDVLKKKHASGGYREMVIYVEACESGSLFEGIMPKDLNVFVTTASNAQENSWVTYCPGTEPSPPPEYTTCLGDLYSVAWMEDSESHNLRRETVNQQYRSVKERTSNFKDYAMGSHVMQYGDTNITAEKLYLFQGFDPATVNLPPHNGRIEAKMEVVHQRDAELLFMWQMYQRSNHLLGKKTHILKQIAETVKHRNHLDGSVELIGVLLYGPGKGSPVLQSVRDPGLPLVDNWACLKSMVRVFESHCGSLTQYGMKHMRAFANICNSGVSESSMEEACMVACGGHDAGHL

SEQ ID NO: 3 - protein, 480 aa, Clitoria ternatea
  source 1..480: note = Sequence comprises His tag, G252V and P183A mutations
MGHHHHHHSSGVDLGTENLYFQSMARLNPQKEWDSVIRLPTEPVDADTDEVGTRWAVLVAGSNGYENYRHQADVCHAYQLLIKGGLKEENIVVFMYDDIAWHELNPRPGVIINNPRGEDVYAGVPKDYTGEDVTAENLFAVILGDRSKVKGGSGKVINSKPEDRIFIFYSDHGGAGVLGMPNEQILYAMDFIDVLKKKHASGGYREMVIYVEACESGSLFEGIMPKDLNVFVTTASNAQENSWVTYCPGTEPSPPPEYTTCLGDLYSVAWMEDSESHNLRRETVNQQYRSVKERTSNFKDYAMGSHVMQYGDTNITAEKLYLFQGFDPATVNLPPHNGRIEAKMEVVHQRDAELLFMWQMYQRSNHLLGKKTHILKQIAETVKHRNHLDGSVELIGVLLYGPGKGSPVLQSVRDPGLPLVDNWACLKSMVRVFESHCGSLTQYGMKHMRAFANICNSGVSESSMEEACMVACGGHDAGHL

SEQ ID NO: 4 - protein, 483 aa, Viola yedoensis
MQLFAAGVILFFLLALSGTIAGGLDVDSLQLPSEAAKFFHNDNSTNDDDSIGTRWAVLIAGSKGYHNYRHQADVCHMYQILRKGGVKDENIIVFMYDDIAYNESNPFPGIIINKPGGENVYKGVPKDYTGEDINNVNFLAAILGNKSAIIGGSGKVLDTSPNDHIFIYYADHGAPGKIGMPSKPYLYADDLVDTLKQKAATGTYKSMVFYVEACNAGSMFEGLLPEGTNIYAMAASNSTEGSWITYCPGTPDFPPEFDVCLGDLWSITFLEDCDAHNLRTETVHQQFELVKKKIAYASTVSQYGDIPISKDSLSVYMGTDPANDNRTFVDENSLRPPLKVIHQHDADLYHIWCKYNMAPEGSSKKIEAQKQLLELMSHRAHVDNSITLIGKLLFGVNKASKVLNTVRPVGQPLVDDWQCLKAMIRTFETHCGSLSEYGMKHTLSFANMCNAGIQKEQLAEAAAQACVTFPSNPYSSLAEGFSA

SEQ ID NO: 5 - protein, 449 aa, Viola yedoensis
MQLFAAGVILFFLLALSGTIAGGLDVDSLQLPSEAAKFFHNDNSTNDDSSAGTKWAVLIAGSKGYQNYRHQADVCHAYQILRRGGVKDENIIVFMYDDIAYDIRNPYPGTITNSPDKKDVYKGVPKDYTGEDVNVQNFLAVILGNKTALTGGSGKVLDTRPNDHIFIYYTDHGYAGVLGMPTQPYLYANDLIDTLKKKHASGTYESLVFYVEACESASIFEGLLPDGLNIYVSTAAKAGEGSWVVYCPTQQPPVPAEYGTCVGDLYSVTWMEDCDLYNLRTQTLHQQYEMVKKKIAYASTVSQFGDLTITKDSLFEYMGTDPANEKHHYEDQENSLRPHVDAVHQREADLYHFWDKYQKASEGSRNKVAARKQLVEVMLHRMHVDDSIESIAKLLFGSDAKASEMMNTIRPPGQPLVSDWDCLKTMVRTFETHCGSLSEYGMKYTRFLA

SEQ ID NO: 6 - protein, 537 aa, Oldenlandia affinis
  source 1..537: note = sequence comprises His tag, C247A mutation
MGMAHHHHHHMQIFVKTLTGKTITLEVEPSDTIENVKAKIQDKEGIPPDQQRLIFAGKQLEDGRTLSDYNIQKESTLHLVLRLRGGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWAYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 7 - protein, 481 aa, Afrohybanthus enneaspermus
MKLLVPGVLLLFLLALSGIAAGRPDDFLRLPSEAAKSFLHNDDDSVGTRWAVLIAGSKGWQNYRHQADVCHAYQILKKGGLKDENIIVFMYDDIAYNESNPRPGIVINKPKGEDVYKGVPKDYTGENVNAVNFLAVLLANRSALTGGSGKVLDSGPNDRIFIYYTDHGAPVTIGMPSKPYLVAKDLVDTLKKKHAAGTYKSMVFYIESCESGSMFDGLLPEDANIYGMTATNSTEGSWVTYCPGQTDDYPEDDEYDVCFGDLWSVAWLEDCDAHNLRTETLDQQYEVVKKKIEYAHIPAQYGNVSLAKDSLFVYMGTDPANDNKTFVEENTLRRPLKAVHSRDADLLHFWHKYHKAPEGTSRKIDAQKQLVEVLSHRTHVDNSIKLVGELLFGVGKASEVLNTIRPAGQPLVDDWDCLKTMVRTFETHCGSLSEYGMKHMRSFANMCNAGVQKEQMAVAAGQACVTFPSNPWSSLDEGFSV

SEQ ID NO: 8 - protein, 455 aa, Arabidopsis thaliana
  source 1..455: note = Sequence comprises His tag
SLEHHHHHHENLYFQGVGTRWAVLVAGSSGYGNYRHQADVCHAYQILRKGGLKEENIVVLMYDDIANHPLNPRPGTLINHPDGDDVYAGVPKDYTGSSVTAANFYAVLLGDQKAVKGGSGKVIASKPNDHIFVYYAXHGGPGVLGMPNTPHIYAADFIETLKKKHASGTYKEMVIYVEAAESGSIFEGIMPKDLNIYVTTASNAQESSYGTYCPGMNPSPPSEYITCLGDLYSVAWMEDSETHNLKKETIKQQYHTVKMRTSNYNTYSGGSHVMEYGNNSIKSEKLYLYQGFDPATVNLPLNELPVKSKIGVVNQRDADLLFLWHMYRTSEDGSRKKDDTLKELTETTRHRKHLDASVELIATILFGPTMNVLNLVREPGLPLVDDWECLKSMVRVFEEHCGSLTQYGMKHMRAFANVCNNGVSKELMEEASTAACGGYSEARYTVHPSILGYSA

SEQ ID NO: 9 - protein, 484 aa, Viola uliginosa
MKLLAAGVILVSLLALSGTVAGGLDVDPLRLPSEAAKFFHNDNSTNDDDSIGTRWAVLIAGSKDYHNYRHQADVCHMYQILRKGGVKDENIIVFMYDDIAYNESNPHPGIIINKPGGEDVYKGVPKDYTGEDVNNINFLAAILGNKSAIIGGSGKVLDTSPNDHIFIYYTDHGAPGKIGMPSKPYLYADDLVDTLKQKAATGTYKSMVFYVEACNAGSMFEGLLPEGTNIYAMAASNSTEGSWITYCPGATPDFPPEYDICLGDLWSITFLEDCDAHNLRTETVHQQFELVKKNIAYASTVSQYGDIPISKDSLSVYMGTDPANDNRTFVDENSLKPPLKVIHQRDADLYHLWYKYNKAPEGSSKKIEAQKQLLELMSHRAHVDNSITLIGKLLFGVDKASKVLNTVRPVGQPLVDDWQCLKAMIRTFETHCGSLSEYGMKHTLSFANMCNAGIQKEQLAEAAAQACVTFPSNSYSSLAEGFSA

SEQ ID NO: 10 - protein, 487 aa, Helianthus annuus
MACFSYRLICLLLVLMMVMALPNGAAAARRGSDYWDPFIRSPVDLEDDELGNGTRWALLVAGSKGYQSYRHQANVCHAYQILKRGGLKDENIVVFMYDDIATCDENPRPGTIIHHPEGGDVYAGVPKDYTGDAVTADNFFAVILGDKSSVKGGSGKVIDSKPDDRIFLYYTDHGAAGLLGMPEKPYVVANDFVEVLKKKHAMGTYKEMVIYLEACESGSIFEGLLPEDLNIYAITSTKPEEPSYIIYCPDMNPPPPPEYTTCLGDTFSVAWMEDSETHNLKKESLAQQINKVKERTSMFGTYANGSHVMEYGTKVIKPEKVYLYQGYNPETANLPANRIHFDKKMESVNQRDGDLIYLWQKYKRSSVSNRAEALKQMTETLRYMAHLDSSVDMIGVLLFGPQNGGSILRSSRGRGLPLVDDWDCLKSMTRLFEKHCGLLTEYGMKHMRAFANICNNLVEETEVEEAIIATCSGKNIGPYASLGAYSV

SEQ ID NO: 11 - protein, 537 aa, Oldenlandia affinis
  source 1..537: note = sequence comprises His tag
MGMAHHHHHHMQIFVKTLTGKTITLEVEPSDTIENVKAKIQDKEGIPPDQQRLIFAGKQLEDGRTLSDYNIQKESTLHLVLRLRGGARDGDYLHLPSEVSRFFRPQETNDDHGEDSVGTRWAVLIAGSKGYANYRHQAGVCHAYQILKRGGLKDENIVVFMYDDIAYNESNPRPGVIINSPHGSDVYAGVPKDYTGEEVNAKNFLAAILGNKSAITGGSGKVVDSGPNDHIFIYYTDHGAAGVIGMPSKPYLYADELNDALKKKHASGTYKSLVFYLEACESGSMFEGILPEDLNIYALTSTNTTESSWCYYCPAQENPPPPEYNVCLGDLFSVAWLEDSDVQNSWYETLNQQYHHVDKRISHASHATQYGNLKLGEEGLFVYMGSNPANDNYTSLDGNALTPSSIVVNQRDADLLHLWEKFRKAPEGSARKEEAQTQIFKAMSHRVHIDSSIKLIGKLLFGIEKCTEILNAVRPAGQPLVDDWACLRSLVGTFETHCGSLSEYGMRHTRTIANICNAGISEEQMAEAASQACASIP

SEQ ID NO: 12 - protein, 339 aa, Homo sapiens
  source 1..339: note = Sequence comprises His tag
VSPSASAWPEEKNYHQPAILNSSALRQIAEGTSISEMWQNDLQPLLIERYPGSPGSYAARQHIMQRIQRLQADWVLEIDTFLSQTPYGYRSFSNIISTLNPTAKRHLVLACHYDSKYFSHWNNRVFVGATDSAVPCAMMLELARALDKKLLSLKTVSDSKPDLSLQLIFFDGEEAFLHWSPQDSLYGSRHLAAKMASTPHPPGARGTSQLHGMDLLVLLDLIGAPNPTFPNFFPNSARWFERLQAIEHELHELGLLKDHSLEGRYFQNYSYGGVIQDDHIPFLRRGVPVLHLIPSPFPEVWHTMDDNEENLDESTIDNLNKILQVFVLEYLHLHHHHHH

SEQ ID NO: 13 - protein, 327 aa, Mus musculus
AWTQEKNHHQPAHLNSSSLQQVAEGTSISEMWQNDLRPLLIERYPGSPGSYSARQHIMQRIQRLQAEWVVEVDTFLSRTPYGYRSFSNIISTLNPEAKRHLVLACHYDSKYFPRWDSRVFVGATDSAVPCAMMLELARALDKKLHSLKDVSGSKPDLSLRLIFFDGEEAFHHWSPQDSLYGSRHLAQKMASSPHPPGSRGTNQLDGMDLLVLLDLIGAANPTFPNFFPKTTRWFNRLQAIEKELYELGLLKDHSLERKYFQNFGYGNIIQDDHIPFLRKGVPVLHLIASPFPEVWHTMDDNEENLHASTIDNLNKIIQVFVLEYLHL

SEQ ID NO: 14 - protein, 340 aa, Drosophila melanogaster [sequence omitted]

SEQ ID NO: 15 - protein, 300 aa, Arabidopsis thaliana
MATRSPYKRQTKRSMIQSLPASSSASSRRRFISRKRFAMMIPLALLSGAVFLFFMPFNSWGQSSGSSLDLSHRINEIEVVAEFPHDPDAFTQGLLYAGNDTLFESTGLYGKSSVRKVDLRTGKVEILEKMDNTYFGEGLTLLGERLFQVAWLTNTGFTYDLRNLSKVKPFKHHMKDGWGLATDGKALFGSDGTSTLYRMDPQTMKVTDKHIVRYNGRESDCIARISPKDGSLLGWILLSKLSRGLLKSGHRGIDVLNGIAWDSDKQRLFVTGKLWPKLYQIKLKQASAKSGNYIEQQCLV

SEQ ID NO: 16 - protein, 345 aa, Conus frigidus
MMEKVTTAATYVRLLLLCSAVASNRALQNLGCGSLTSQYTVDNLSNLTVGMSDDGLRKKALPPLLKPRVSGTRGNFNVRNSIIKWMRREGWSVQEDPFIAKTPYGWVRFSNVIATLNPRAARRVVLACHYDSKLILFHGLSFVGATDSAVPCALLMDSAKKLRQVFQEKVADASFQELTLQFIFFDGEEAYVQWSRSDSLYGARHLAQKWASTPDPTAAGLNYLQTIGVFILLDLIGSADTRFANLFNQTAGVYAKLQSIEMCLTENGYLDATANPLPLFTSEQKQGTIEDDHLPFLRRGVPVVHLISTPFPSVWHKLSDNLHALDFQRTENLARILRLFLVDLL

SEQ ID NO: 17 - protein, 368 aa, Sistrurus tergeminus
MARERRDSKAATFFCLAWTLCLALPGFPQHVSGREDRADWTQEKYSHRPTILNATCILQVTSQTNVNRMWQNDLHPILIERYPGSPGSYAVRQHIKHRLQGLQAGWLVEEDTFQSHTPYGYRTFSNIISTLNPLAKRHLVIACHYDSKYFPPQLDGKVFVGATDSAVPCAMMLELARSLDRQLSFLKQSSLPPKADLSLKLIFFDGEEAFVRWSPSDSLYGSRSLAQKMASTPHPPGARNTYQIQGIDLFVLLDLIGARNPVFPVYFLNTARWFGRLEAIERNLYDLGLLNNYSSERQYFRSNLRRHPVEDDHIPFLRRGVPILHLIPSPFPRVWHTMEDNEENLDKPTIDNLSKILQVFVLEYLNLG

SEQ ID NO: 18 - protein, 267 aa, Xanthomonas campestris
MPRLVPALLLILALLPAMAVARDPVPTQGYRVVKRYPHDTTAFTEGLFYLRGHLYESTGETGRSSVRKVDLETGRILQRAEVPPPYFGEGIVAWRDRLIQLTWRNHEGFVYDLATLTPRARFRYPGEGWALTSDDSHLYMSDGTAVIRKLDPDTLQQVGSIKVTAGGRPLDNLNELEWVNGELLANVWLTSRIARIDPASGKVVAWIDLQALVPDADALTDSTNDVLNGIAFDAEHDRLFVTGKRWPMLYEIRLTPLPHAAAGKHAQ

SEQ ID NO: 19 - protein, 85 aa, synthetic construct
MQIFVKTLTGKTITLEVEPSDTIENVKAKIQDKEGIPPDQQRLIFAGKQLEDGRTLSDYNIQKESTLHLVLRLRGGNQLHHHHHH

SEQ ID NO: 20 - protein, 78 aa, synthetic construct
GIMQIFVKTLTGKTITLEVEPSDTIENVKAKIQDKEGIPPDQQRLIFAGKQLEDGRTLSDYNIQKESTLHLVLRLRGG

SEQ ID NO: 21 - protein, 254 aa, synthetic construct
MGIGSKKVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSGHHHHHH

SEQ ID NO: 22 - protein, 261 aa, synthetic construct
MGIGSGGGSGGGSKKVSKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYKGSHHHHHH

SEQ ID NO: 23 - protein, 175 aa, synthetic construct
MHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTPFDLAIDNGNEDIAEVLQKAAKLGSGSNQL

SEQ ID NO: 24 - protein, 173 aa, synthetic construct
MGISSHHHHHHGSDLGKKLLEAARAGQDDEVRILMANGADVNAKDFYGITPLHLAAAYGHLEIVEVLLKHGADVNAHDWNGWTPLHLAAKYGHLEIVEVLLKHGADVNAIDNAGKTPLHLAAAHGHLEIVEVLLKYGADVNAQDKFGKTPFDLAIDNGNEDIAEVLQKAAKLN

SEQ ID NO: 25 - protein, 81 aa, Synthetic construct
MKKGSSHHHHHHLQVDNKFNKEMWAAWEEIRNLPNLNGWQMTAFIASLVDDPSQSANLLAEAKKLNDAQAPKVDGSGSNQL

SEQ ID NO: 26 - protein, 310 aa, synthetic construct
MKKGSSHHHHHHLQVDNKFNKEMWAAWEEIRNLPNLNGWQMTAFIASLVDDPSQSANLLAEAKKLNDAQAPKVDGSGSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKGSNQL

SEQ ID NO: 27 - protein, 6 aa, synthetic construct
  SITE 1: note = Residue 1 is N-terminally linked to Ac (acetyl group)
SYRNQL

SEQ ID NO: 28 - protein, 6 aa, synthetic construct
  SITE 1: note = Residue 1 is N-terminally linked to Ac (acetyl group)
SYRNGL

SEQ ID NO: 29 - protein, 6 aa, synthetic construct
GIGGIR

SEQ ID NO: 30 - protein, 6 aa, synthetic construct
  SITE 1: note = Residue 1 is N-terminally linked to biotin
GRSNQL

SEQ ID NO: 31 - protein, 7 aa, synthetic construct
  SITE 7: note = Lysine side chain is linked to biotin
GIGGIRK

SEQ ID NO: 32 - protein, 5 aa, synthetic construct
QLGSA

SEQ ID NO: 33 - protein, 5 aa, synthetic construct
QFGSA

SEQ ID NO: 34 - protein, 5 aa, synthetic construct
QIGSA

SEQ ID NO: 35 - protein, 5 aa, synthetic construct
QVGSA

SEQ ID NO: 36 - protein, 6 aa, synthetic construct
  SITE 1: note = Residue 1 is N-terminally linked to Fmoc
  SITE 6: note = Residue 6 is C-terminally linked to PABC-MMAE
GIGKVA

SEQ ID NO: 37 - protein, 6 aa, synthetic construct
  SITE 6: note = Lysine side chain is linked to DOTA-FE3+
GIGGGK

SEQ ID NO: 38 - protein, 11 aa, synthetic construct
  SITE 9: note = side chain of the lysine is linked via an amide linkage to another sequence GISGGRAG
GISGGRAGKGC

SEQ ID NO: 39 - protein, 8 aa, synthetic construct
  SITE 8: note = glycine in position number 8 is linked to another sequence GISGGRAGKGC
GISGGRAG

(19) United States
(12) Patent Application Publication
(10) Pub. No.: US 2025/0270610 A1
(43) Pub. Date: Aug. 28, 2025

(54) Improving the Efficiency of PAL-Catalyzed Protein Ligation By A Cascade Enzymatic Scheme

(71) Applicant: Nanyang Technological University, Singapore (SG)

(72) Inventors: Chuan Fa Liu, Singapore (SG); Yiyin Xia, Singapore (SG); Fupeng Li, Singapore (SG)

(21) Appl. No.: 18/569,054

(22) PCT Filed: Mar. 31, 2023
(86) PCT No.: PCT/SG2023/050219

§ 371 Date: Dec. 11, 2023

(30) Foreign Application Priority Data
Mar. 31, 2022 (SG) 10202203286W
Mar. 1, 2023 (SG) 10202300549P

(51) Int. Cl. C12P 21/02; A61K 47/64; A61K 47/68; A61K 51/08; C12N 9/00; C12N 9/10

(52) U.S. Cl. CPC C12P 21/02; A61K 47/6415; A61K 47/68031; A61K 47/68033; A61K 47/68037; A61K 47/6831; A61K 51/088; C12N 9/104; C12N 9/93; C12Y 203/02005; C12Y 601/01022

(57) ABSTRACT

The present invention generally relates to enzymatic peptide or protein ligation. In particular, the present invention provides an improved method of enzymatic peptide or protein ligation, which comprises coupling a peptidyl asparaginyl ligase (PAL)-catalyzed ligation to a glutaminyl cyclase (QC)-catalyzed pyroglutamyl formation to improve yield of ligated product.

### CROSS-REFERENCE TO RELATED APPLICATIONS

This application is a national phase entry pursuant to 35 U.S.C. § 371 of International Application No. PCT/SG2023/050219, filed Mar. 31, 2023, which claims the benefit of priority of Singapore application Ser. No. 10/202,203286W, filed Mar. 31, 2022, and Singapore application Ser. No. 10/202,300549P, filed Mar. 1, 2023, each of which is incorporated herein by reference in its entirety for any purpose.

### FIELD OF THE INVENTION

The present invention generally relates to enzymatic peptide or protein ligation. In particular, the present invention provides an improved method of enzymatic peptide or protein ligation, which comprises coupling a peptidyl asparaginyl ligase (PAL)-catalyzed ligation to a glutaminyl cyclase (QC)-catalyzed pyroglutamyl formation to improve yield of ligated product.

### BACKGROUND OF THE INVENTION

Site-specific protein ligation facilitates protein function studies and enables the development of protein therapeutics (Spicer, C. D., and Davis, B. G., Nat. Commun. 5:4740 (2014); Hoyt, E. A., et al., Nat. Rev. Chem. 3, 147-171 (2019); Zhang, G., et al., Chem. Soc. Rev. 44, 3405-3717 (2015); Sletten, E. M. and Bertozzi, C. R., Angew. Chem. Int. Ed. 48, 6974-6998 (2009); Rosen, C. B., and Francis, M. B., Nat. Chem. Biol. 13, 697-705 (2017)). Besides the well-established chemical ligation methods, recent years have seen an increasing use of peptide ligases for protein modification (Zhang, Y., et al., Chem. Soc. Rev. 47, 9106-9136 (2018); Lotze, J., et al., Mol Biosyst. 12, 1731-1745 (2016); Weeks, A. M. and Wells, J. A., Chem. Rev. 120, 3127-3160 (2020); Pishesha, N., et al., L. Annu. Rev. Cell Dev. Biol. 34, 163-88 (2018)). For example, a popular peptide ligase has been sortase A, which recognizes a sorting sequence LPXTG and ligates after Thr (Pishesha, N., et al., L. Annu. Rev. Cell Dev. Biol. 34, 163-88 (2018)). Nevertheless, the catalytic efficiency of sortase A is low, requiring a stoichiometric amount of the enzyme for a practicable ligation reaction. In contrast, the recently discovered peptidyl asparaginyl ligases (PALs) as represented by butelase-1 (Nguyen, G. K. T., et al., Nat Chem Biol. 10, 732-738 (2014)), have a catalytic efficiency that is 10⁴ times that of sortase A (Nguyen, G. K. T., et al., Nat. Protoc. 11, 1977-1988 (2016) Tam, J. P., et al., Science China Chemistry, 63, 296-307 (2020)). Furthermore, PALs require a minimal asparaginyl tripeptide recognition motif, Asn-P1′-P2′, for ligation after Asn, where P1′ can tolerate a broad range of amino acid residues and P2′ is usually a large hydrophobic residue (Nguyen, G. K. T., et al., Nat. Protoc. 11, 1977-1988 (2016); Tam, J. P., et al., Science China Chemistry, 63, 296-307 (2020)). As members of the asparaginyl endopeptidase (AEP) family, PALs utilizes the catalytic cysteinyl thiol to cleave the Asn-P1′ peptide bond in an acyl donor substrate, and the resultant asparaginyl thioester intermediate is then resolved by the amine nucleophile of an acyl acceptor substrate. Therefore, the ligation product is formed through transpeptidation. Like butelase-1, VyPAL2 and OaAEP1b-C247A are two other PALs that have excellent transpeptidase activity (Hemua, X., et al., Proc. Natl. Acad. Sci. 116, 11737-11746 (2019); Harris, K. S., et al., Nat. Commun. 6, 10199 (2015); Yang, R., et al., J. Am. Chem. Soc. 2017, 139, 5351-5358 (2017)). However, since transpeptidation is reversible, an excess amount of the incoming nucleophile is needed for a high-yielding intermolecular ligation reaction.

Several methods have been developed to shift the equilibrium of the PAL-mediated ligation to the product side (Nguyen, G. K. T., et al., Angew. Chem. Int. Ed. 54, 15694-15698 (2015); Cao, Y., et al., Bioconj. Chem. 27, 2592-2596 (2016); Rehm, F. B. H., et al., J. Am. Chem. Soc. 141 (43), 17388-17393 (2019); Tang, T. M. S., et al., Chem. Sci. 11, 5881-5888 (2020); Rehm, F. B. H., et al., Angew. Chem. Int. Ed. 60, 4004-4008 (2021)). The thiodepsipeptide method was first developed which utilizes an asparaginyl thioester peptide as the acyl donor substrate (peptide-Asn-thioglc-Xaa) to make the ligation reaction irreversible (Nguyen, G. K. T., et al., Angew. Chem. Int. Ed. 54, 15694-15698 (2015); Cao, Y., et al., Bioconj. Chem. 27, 2592-2596 (2016)). Nevertheless, a limitation of this method lies with the need to prepare the thioester substrates. In a second method developed by Rehm et al., a glycinyl-valinyl acyl acceptor substrate was used to ligate with an NGL-containing acyl donor as the resultant Asn-Gly-Val (NGV) motif was more stable than Asn-Gly-Leu (NGL) toward OaAEP-C247A, which reduced both product hydrolysis and reversibility of the ligation reaction. However, NGV is only relatively more stable than NGL. As a poorer acceptor substrate, the GV-peptide must be used in a large excess (>20-fold) to the acyl donor. No protein-protein ligation was demonstrated. Another method, developed by Tang et al., used Asn-Cys-Leu as the P1-P1′-P2′ tripeptide motif for OaAEP1-C247A (Tang, T. M. S., et al., Chem. Sci. 11, 5881-5888 (2020)). Quenching the N-terminal cysteine of the Cys-Leu leaving group by the aldehyde compound, 2-formyl phenylboronic acid (FPBA), reduced reversibility of the ligation reaction. However, FPBA was found to slow down the ligation reaction significantly, likely because the aldehyde could form imines with amine substrates and/or the enzyme. Lastly, a method reported by Rehm et al. made use of Ni²⁺ to chelate a GLH tripeptide leaving group to mask the nucleophilicity of its N-terminal amine (Rehm, F. B. H., et al., Angew. Chem. Int. Ed. 60, 4004-4008 (2021). This approach gave a great increase in the yield of protein N- and C-terminal labelling reactions with small synthetic peptides. However, for protein-to-protein ligations, only a modest yield increase was observed-from 24% to 36% in one case and 28% to 39% in another (Rehm, F. B. H., et al., Angew. Chem. Int. Ed. 60, 4004-4008 (2021). Therefore, all these currently known methods have certain limitations, especially when protein-protein ligation is concerned.

Accordingly, there is a need to provide improved methods of protein ligation that overcome or at least ameliorate, one or more of the drawbacks described above.

### SUMMARY OF THE INVENTION

The present invention relates to a method of enzymatic peptide ligation in which PAL-mediated intermolecular ligation is coupled to glutaminyl cyclase (QC)-catalyzed pyroglutamyl formation to significantly increase the yield of ligated product.

In one aspect, there is provided a method of enzymatic peptide ligation, said method comprising providing
i) a peptidyl asparaginyl ligase (PAL) and a glutaminyl cyclase (QC);
ii) a first peptide or protein having a P1-P1′-P2′ tripeptide PAL motif as an acyl donor, wherein P1 is Asn or Asp, P1′ is Gln or Glu and P2′ is a hydrophobic amino acid or a β-branched amino acid;
iii) a second peptide or protein which may be the same or different to the first peptide or protein, having a P1″-P2″ motif as an acyl acceptor at the N-terminus, wherein P1″ is any amino acid and P2″ is a hydrophobic amino acid or a B-branched amino acid;
iv) contacting the peptidyl asparaginyl ligase (PAL) and the glutaminyl cyclase (QC) with said first and second peptides/proteins;
   wherein PAL cleaves the first peptide/protein after P1 in the tripeptide PAL motif and ligates said first peptide/protein to the P1″-P2″ motif of said second peptide/protein, and QC cyclizes P1′ in the released P1′-P2′ dipeptide motif to pyroglutamyl (pGlu).

It would be understood that peptides are generally shorter than proteins, comprise amino acids, and are both suitable for ligation according to the invention. In some embodiments, the P2′ is selected from the group comprising Leu, Met, Phe, Tyr, Trp, Val, Ile and Thr.

In some embodiments, the P2″ is selected from the group comprising Leu, Phe, Tyr, Trp, Val, Ile and Thr.

In some embodiments, the PAL is selected from the group comprising butelase1 comprising the amino acid sequence set forth in SEQ ID NO: 1, butelase-2 comprising the amino acid sequence set forth in SEQ ID NO: 2 or SEQ ID NO: 3, VyPAL2 comprising the amino acid sequence set forth in SEQ ID NO: 4, VyPAL3 comprising the amino acid sequence set forth in SEQ ID NO: 5, OaAEP1b-C247A comprising the amino acid sequence set forth in SEQ ID NO: 6, HeAEP3 comprising the amino acid sequence set forth in SEQ ID NO: 7, AtLEGY comprising the amino acid sequence set forth in SEQ ID NO: 8, VuPAL1 comprising the amino acid sequence set forth in SEQ ID NO: 9, HaPAL1 comprising the amino acid sequence set forth in SEQ ID NO: 10 and OaAEP1b comprising the amino acid sequence set forth in SEQ ID NO: 11.

In some embodiments, the QC is selected from the group comprising Human glutaminyl cyclase comprising the amino acid sequence set forth in SEQ ID NO: 12, Mouse glutaminyl cyclase comprising the amino acid sequence set forth in SEQ ID NO: 13, Drosophila glutaminyl cyclase comprising the amino acid sequence set forth in SEQ ID NO: 14, Arabidopsis glutaminyl cyclase comprising the amino acid sequence set forth in SEQ ID NO: 15, Conus glutaminyl cyclase comprising the amino acid sequence set forth in SEQ ID NO: 16 Sistrurus glutaminyl cyclase comprising the amino acid sequence set forth in SEQ ID NO: 17, and Bacterial glutaminyl cyclase comprising the amino acid sequence set forth in SEQ ID NO: 18.

In some embodiments, the second peptide or protein further comprises a spacer of at least one amino acid between the P1″-P2″ acyl acceptor and said second peptide/protein.

In some embodiments, the ratio of QC: PAL: first peptide or protein is in the range of 0.1:1:1000 to 1:1:50, respectively, preferably in the range of 0.1:1:1000 to 0.1:1:50, respectively.

In some embodiments, the said first and second peptides or proteins are the same and form a dimer upon ligation.

In some embodiments, one of said first and second peptides or proteins is an epitope-binding peptide or protein and the other peptide or protein comprises a payload.

In some embodiments, the payload further comprises a payload-releasing linkage.

In some embodiments, the epitope-binding peptide or protein is selected from the group comprising an antibody or functional fragment thereof, an affibody such as ZEGFR Or ZEGFR-FC, and DARPin.

In some embodiments, the antibody or functional fragment thereof is selected from the group comprising minibody, diabody, scFv, nanobody and F(ab′)₂.

In some embodiments, the payload is an imaging agent or a therapeutic agent.

In some embodiments, the imaging agent is a radiolabel chelator or an optical label.

In some embodiments, the radiolabel chelator is selected from the group comprising 1,4,7-triazacyclononanetriacetic acid (NOTA), 1,4,7,10-tetraazacyclododecane-1,4,7,10-tetraacetic acid (DOTA) and 1,4,7-triazacyclononane-1-glutaric acid-4,7-diacetic acid (NODAGA) and/or the optical label is HRP or GFP or the like.

In some embodiments, the therapeutic agent is Monomethyl auristatin E (MMAE) or radiolabelled DOTA.

Advantageously, the method of the present disclosure allows protein-to-protein, protein-peptide, and peptide-peptide ligation to be conducted in a much greater efficiency, and can achieve near-quantitative yields even at an equal molar ratio between the two ligation partners. These and other advantages of the present disclosure will become readily apparent to those skilled in this art from the following detailed description.

### BRIEF DESCRIPTION OF THE FIGURES

The accompanying drawings illustrate disclosed embodiments and serve to explain the principles of the disclosed embodiments. It is to be understood, however, that the drawings are designed for purposes of illustration only, and not as a definition of the limits of the invention.

FIG. 1 shows a model study using the ligation reaction between Ac-SYRNQL and GIGGIR as a proof of concept for the cascade enzymatic reaction scheme. (a) The product yield as a function of time when the reaction was conducted in the absence (filled triangles) or presence of QC (filled circles for 0.1 eq QC to PAL and filled squares for 1 eq QC to PAL). Reaction conditions: 5 mM acyl acceptor and 5 mM acyl donor, OaAEP1b-C247A (0.0005 eq) in 20 mM PBS (pH 7) at 37° C., with or without QC (0.0005 or 0.00005 eq). The yields were determined by HPLC (UV absorbance at 220 nm). Yields are presented as means±SEM from triplicate experiments (b) HPLC traces. Upper trace: Ac-SYRNQL and GIGGIR only; middle trace: ligation reaction without QC; lower trace: ligation reaction with QC. Reaction time=32 min.

FIG. 2 shows the use of PAL-QC coupled scheme for protein-peptide ligation. (a) Ligation between ubiquitin-NQL-His₆ and GIGGIRK (biotin); (b) ligation between biotin-GRSNQL and Gl-ubiquitin. Both reactions were conducted at 37° C. using 500 μM of ubiquitin, 1.2 eq of peptide, 0.001 eq of OaAEP1b-C247A with or without 0.0001 eq of QC in 20 mM PBS (pH 7) for 1 h. Both reactions were monitored using HPLC (middle panel) and the labelling products were characterized by ESI-MS (lower panel).

FIG. 3 shows the use of the PAL-QC coupled cascade scheme for preparing C-terminal conjugates of the affibody protein Z_{EGFR}. (a) Ligation of Z_{EGFR}-NQL with GIGGGK[Fe(DOTA)]; (b) Ligation of Z_{EGFR}-NQL with GIGKVA-PABC-MMAE. The reactions were conducted at 23° C. using 500 μM of Z_{EGFR}-NQL and 1.5 eq of the nucleophile substrate and 0.001 eq of OaAEP1b-C247A with or without 0.0001 eq of QC in 20 mM PBS (pH 7) for 2 h. (c) Preparation of a C-terminal dimer of Z_{EGFR}. 1.1 mM of Z_{EGFR}-NQL was reacted with 500 μM of the bivalent peptide (GISGGRG)₂KGC, 2 μM of OaAEP1b-C247A with or without 0.2 μM of QC in 20 mM PBS (pH 7) at 23°° C. for 2 h. Reactions were monitored using HPLC and ESI-MS.

FIG. 4 depicts enhancement of protein-protein ligation efficiency by coupled use of VyPAL2 with QC. (a) HPLC monitoring of VyPAL2-mediated ligation between DARPin-NQL and GI-ubiquitin with or without QC. (b) Yields of various protein-protein ligations in the presence or absence of QC (i: DARPin-NQL and Gl-ubiquitin, ii: DARPin-NQL and GI-DARPin, iii: Z_{EGFR}-NQL and Gl-ubiquitin, iv: DARPin-QL and GI-GFP, v: DARPin-NQL and GI-GGGSGGGS-GFP). All reactions were conducted at 37° C. using 400 μM of acyl donor protein, 1.8 eq of acyl acceptor protein, 0.001 eq of VyPAL2 with/without 0.0001 eq of QC in 20 mM PBS (pH 7) for 3-4 h. The reactions were monitored using HPLC and ESI-MS (FIGS. 13-18). The yields in b were calculated based on the integrated areas of the reactant and product peaks in HPLC and are represented as means±SEM from triplicated experiments.

FIG. 5 depicts Ligation between Z_{EGFR}-Fc-NQL and GI-GGGSGGGS-GFP. (a) SDS-PAGE of the ligation reaction of Z_{EGFR}-Fc-NQL (200 μM) with GI-GGGSGGGS-GFP (500 μM) using 0.4 μM VyPAL2 with or without 0.04 μM QC in 20 mM PBS (pH 7). The reaction mixture was treated with 50 mM DTT and analyzed by SDS-PAGE gel. The bands at ˜63 kDa and 34 kDa correspond to the reduced forms of the ligation product and Z_{EGFR}-Fc-NQL, respectively. The ligation product was also characterized by LC-MS (ESI) after reduction with DTT. (b). Confocal microscopy image of Z_{EGFR}-Fc-GFP binding to A431 cells (c). Flow cytometry analysis of Z_{EGFR}-FC-GFP and GFP targeting A431 cells.

FIG. 6 shows the ligation between Z_{EGFR}-Fc-NQL and GVA-PABC-MMAE. Left panel: without QC. Right panel: with QC.

FIG. 7 shows QC-catalyzed pyro-glutamate (pGlu) formation for 4 substrates (QFGSA, QLGSA, QIGSA and QVGSA). The reactions were performed using 5 mM of QXGSA, 0.0001 eq of QC at 37° C. for 15 min in 20 mM PBS (pH 7) and monitored by RP-HPLC. All Bar charts represent mean±SEM from triplicated measurements.

FIG. 8 shows the yields from ligation between Ac-SYRNQL and GIGGIR catalyzed by different PALs (OaAEP1b-C247A, VyPAL2 or butelase-1) in the absence or presence of QC. The ligation reactions were performed using 5 mM Ac-SYRNQL, 5 mM GIGGIR, 0.0005 eq of PAL, with or without 0.00005 eq of QC, at 37° C. for 30 min in 20 mM PBS PH 7 (OaAEP1b-C247A) or pH 6.5 (VyPAL2, butelase-1). All Bar charts represent mean±SEM from triplicated measurements.

FIG. 9 depicts RP-HPLC monitoring of the ligation reaction between Z_{EGFR}-NQL and GIGGGK[Fe(DOTA)] at 2 h. The ligated product was analyzed using ESI-MS. The ligation reaction was performed at 37° C. using 500 μM Z_{EGFR}-NQL, 1.5 eq of GIGGGK[Fe(DOTA)], 0.001 eq of VyPAL2 in 20 mM PBS (pH 7), in the absence or presence of 0.0001 eq of QC.

FIG. 10 depicts RP-HPLC monitoring of the ligation reaction between Z_{EGFR}-NQL and GIGKVA-PABC-MMAE. The ligated product was analyzed using ESI-MS. The ligation reaction was performed at 37° C. for 2 h using 500 μM Z_{EGFR}-NQL, 1.5 eq of GIGKVA-PABC-MMAE, 0.001 eq of VyPAL2 in 20 mM PBS (pH 7), in the absence or presence of 0.0001 eq of QC.

FIG. 11 depicts RP-HPLC monitoring of the ligation reaction between Z_{EGFR}-NQL and (GISGGRAG)₂KGC, a bivalent peptide. The ligated product was analyzed using ESI-MS. The ligation reaction was performed at 37° C. for 2 h using 1.1 mM Z_{EGFR}-NQL, 500 μM of (GISGGRAG)₂KGC, 2 μM of VyPAL2 in 20 mM PBS (pH 7), in the absence or presence of 0.2 μM of QC.

FIG. 12 shows the IC₅₀ of the Z_{EGFR}-MMAE conjugate against A431 cells and MCF7 cells under 3 days treatment with Z_{EGFR}-PABC-MMAE. IC₅₀ against A431 cells ˜12.9 nM; IC₅₀ against MCF7 cells >100 nM. To test the viability of A431 and MCF-7 cells toward the Z_{EGFR}-MMAE conjugate, ˜5000 A431 or MCF-7 cells were seeded separately on a 98-well plate and incubated in the medium at 37° C. under 5% CO₂ overnight. Z_{EGFR}-MMAE at different concentrations were added and incubation continued at 37° C. under 5% CO₂ for 3 days. Cell viability was measured by the MTT assay following recommended protocols.

FIG. 13 depicts RP-HPLC monitoring of the ligation reaction between DARPin-NQL and GI-ubiquitin at different time points. The ligated product was analyzed using ESI-MS. The ligation reaction was performed at 37° C. using 400 μM DARPin-NQL, 1.8 eq of GI-ubiquitin, 0.001 eq of VyPAL2 in 20 mM PBS (pH 7), in the absence or presence of 0.0001 eq of QC.

FIG. 14 depicts RP-HPLC monitoring of the ligation reaction between DARPin-NQL and GI-GFP at different time points. The ligated product was analyzed using ESI-MS. The ligation reaction was performed at 37° C. using 400 μM DARPin-NQL, 1.8 eq of GI-GFP, 0.001 eq of OaAEP1b-C247A and 0.0001 eq of QC in 20 mM PBS (pH 7). The reaction led to about 15% of the hydrolysis product DARPin-N-OH (labelled as H).

FIG. 15 depicts RP-HPLC monitoring of the ligation reaction between DARPin-NQL and GI-DARPin at different time points. The ligated product was analyzed using ESI-MS. The ligation reaction was performed at 37° C. using 400 μM DARPin-NQL, 1.8 eq of GI-DARPin, 0.001 eq of VyPAL2 in 20 mM PBS (pH 7), in the absence or presence of 0.0001 eq of QC.

FIG. 16 shows RP-HPLC monitoring of the ligation reaction between Z_{EGFR}-NQL and GI-ubiquitin at different time points. The ligated product was analyzed using ESI-MS. The ligation reaction was performed at 37° C. using 400 μM Z_{EGFR}-NQL, 1.8 eq of GI-ubiquitin, 0.001 eq of VyPAL2 in 20 mM PBS (pH 7), in the absence or presence of 0.0001 eq of QC. *peak=the [Z_{EGFR}]₂-ubi by-product formed by the addition of an extra Z_{EGFR} onto the N-terminus of the desired product.

FIG. 17 shows RP-HPLC monitoring of the ligation reaction between DARPin-NQL and GI-GFP at different time points. The ligated product was analyzed using ESI-MS. The ligation reaction was performed at 37° C. using 400 μM DARPin-NQL, 1.8 eq of GI-GFP, 0.001 eq of VyPAL2 in 20 mM PBS (pH 7), in the absence or presence of 0.0001 eq of QC. Peak H=hydrolysis product DARPin-N-OH.

FIG. 18 shows RP-HPLC monitoring of the ligation reaction between DARPin-NQL and GI-linker-GFP at different time points. The ligated product was analyzed using ESI-MS. The ligation reaction was performed at 37° C. using 400 μM DARPin-NQL, 1.8 eq of GI-GGGSGGGS-GFP, 0.001 eq of VyPAL2 in 20 mM PBS (pH 7), in the absence or presence of 0.0001 eq of QC.

FIG. 19 shows the results of monitoring the ligation reaction between Z_{EGFR}-Fc-NQL and GI-GGGSGGGS-GFP at different time points by non-reducing SDS-PAGE gel. The ligation reaction was performed at 37° C. using 200 μM Z_{EGFR}-Fc-NQL, 500 μM GI-GGGSGGGS-GFP, and 0.4 μM VyPAL2 in 20 mM PBS (pH 7), in the absence or presence of 0.04 μM QC.

FIG. 20 depicts the fluorescence intensity (measured by flow cytometry, λex=488 nm) of untreated A431 and MCF7 cells (ctrl) shown in dark grey fill (Blank), and cells treated with either GFP (100 nM, 30 min), or with Z_{EGFR}-Fc-GFP (100 nM, 30 min) (shown by arrow), respectively. Forward scatter (FSC-A) versus side scatter (SSC-A) were used to gate intact cells.

FIG. 21 shows confocal fluorescence microscopy images of EGFR-positive A431 cells after incubating with Z_{EGFR}-Fc-GFP or GFP. Z_{EGFR}-FC-GFP staining of the membrane was much brighter than that for GFP when the top left panel of each block is compared. The plasma membrane was stained with PKH26 red-fluorescent dye and is shown in the lower left panel and merged image of each block. Scale bar=20 μm.

FIG. 22 depicts the mass spectra of protein substrates of the present invention.

FIG. 23 depicts the structure of Gly-Val-Ala-PABC-MMAE (or GVA-PABC-MMAE).

FIG. 24 shows the structures of payload drug examples in which the amine group can be modified with a linker for ligation by PALs as shown in FIGS. 25, 26, and 29.

FIG. 25 shows the structures of examples of the linker-payload compounds as acyl acceptor substrates for PAL-mediated ligation with monoclonal antibodies or other proteins. There is a payload-releasing linkage in these compounds.

FIG. 26 depicts the structures of examples of the linker-payload compounds as acyl acceptor substrates for PAL-mediated ligation with monoclonal antibodies or other proteins. These compounds do not have a payload-releasing linkage.

FIG. 27 depicts the structures of payload drug examples in which the hydroxyl group can be modified with a linker for ligation by PALs as shown in FIGS. 28 and 30.

FIG. 28 shows the structures of examples of linker-payload compounds as acyl acceptor substrates for ligation with proteins and monoclonal antibodies by PALs.

FIG. 29 depicts the structure of a bivalent drug-linker compound as an acyl acceptor substrate for ligation with proteins and monoclonal antibodies by PALs. The drug payload is linked to the bivalent linker through PABC via an amino group in the drug.

FIG. 30 depicts the structure of a bivalent drug-linker compound as an acyl acceptor substrate for ligation with proteins and monoclonal antibodies by PALs. The drug payload is linked to the bivalent linker through its hydroxyl group.

FIG. 31 depicts a ligation reaction between Ac-SYRNQL and GIGGIR using mouse QC and OaAEP1b. Reaction conditions: 5 mM acyl acceptor and 5 mM acyl donor, OaAEP1b-C247A (0.0005 eq or 0.05 mol %) in 20 mM PBS (pH 7) at 37° C., with QC (0.00005 eq). In the absence of QC, the reaction gave the ligation product in about 45% yield (see FIG. 1). The yields were determined by HPLC (UV absorbance at 220 nm).

FIG. 32 shows a schematic diagram of a method of the present invention. (A) PAL-catalyzed intermolecular ligation is a reversible reaction; (B) Coupling QC with PAL forms a cascade enzymatic reaction scheme which overcomes the reversibility problem of PAL-mediated ligation.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

### Definitions

For convenience, certain terms employed in the specification, examples and appended claims are collected here.

In general, technical, scientific and medical terminologies used herein has the same meaning as understood by those skilled in the art to which this invention belongs. Further, the following technical comments and definitions are provided. These definitions should in no way limit the scope of the present invention to those terms alone, but are put forth for a better understanding of the following description.

As used herein, “a” or “an” may mean one or more than one unless indicated to the contrary or otherwise evident from the context. The inventors found that the more enzyme used the faster the reaction proceeded.

As used herein, the term “amino acid” may refer to natural and/or unnatural or synthetic amino acids, including both the D and L optical isomers, amino acid analogs (for example norleucine is an analog of leucine) and peptidomimetics. As used in the context of the present application, the term “amino acid” typically refers to the 20 naturally occurring L-amino acids, namely Gly, Ala, Val, Leu, He, Phe, Cys, Met, Pro, Thr, Ser, Glu, Gln, Asp, Asn, His, Lys, Arg, Tyr, and Trp.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “functional fragment” refers to a portion of a protein that retains some or all of the activity or function (e.g., biological activity or function, such as enzymatic activity) of the full-length protein, such as, e.g., the ability to catalyse a ligation reaction between two peptide. The functional fragment can be any size, provided that the fragment retains the activity/functionality of the full-length protein/enzyme.

As used herein, the terms “peptide”, “polypeptide” and “protein” are used interchangeably to denote a polymer of at least two amino acids covalently linked by an amide bond. Whereas peptides are considered to be short amino acid chains, polypeptides are long amino acid chains and proteins tend to have a stable structure and may comprise modifications (e.g., glycosylation or phosphorylation). The term “protein” may encompass a naturally-occurring as well as artificial (e.g., engineered or variant) full-length protein as well as a functional fragment of the protein. It would be understood that, for the purpose of the invention, any combination of peptide, polypeptide or protein may be ligated in a reaction using PAL and QC providing one has a PAL acyl donor and the other has an acyl acceptor.

As used herein, the term “QC” refers to glutaminyl cyclase (QC) enzyme and QC-like enzymes. QC and QC-like enzymes have identical or similar enzymatic activity, i.e., catalysing the intramolecular cyclization of N-Terminal glutaminyl and glutamyl residues of peptides and proteins to form pyroglutamyl residue (pGlu). In this regard, QC-like enzymes can fundamentally differ in their molecular structure from QC.

As used herein, the term “variant”, refers to an amino acid sequence that is altered by one or more amino acids of the non-variant reference sequence, but retains the ability to recognize its target and affect its function. For example, a QC peptide variant is altered by one or more amino acids of the non-variant QC peptide reference sequence, but retains the ability to catalyse the intramolecular cyclization of N-Terminal glutaminyl and glutamyl residues of peptides and proteins to form pyroglutamyl residue (pGlu). The variant may have “conservative” changes, wherein a substituted amino acid has similar structural or chemical properties (e.g., replacement of leucine with isoleucine). More rarely, a variant may have “non-conservative” changes (e.g., replacement of glycine with tryptophan). Analogous minor variations may also include amino acid deletions or insertions, or both. Guidance in determining which amino acid residues may be substituted, inserted, or deleted without abolishing biological activity may be found using computer programs well known in the art, for example, DNASTAR® software (DNASTAR, Inc. Madison, Wisconsin, USA).

A description of exemplary, non-limiting embodiments of the invention follows.

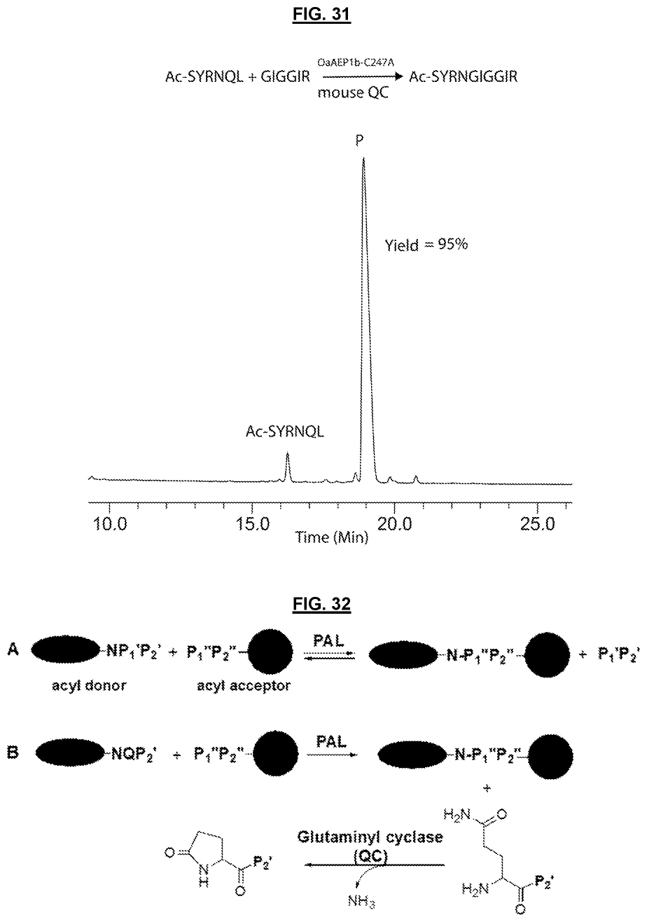

The present invention provides an improved method of peptide ligation. In this regard, the present invention is based, in part, on the inventors' discovery that coupling QC with PAL forms a cascade enzymatic reaction scheme which overcomes the reversibility problem of PAL-mediated ligation (see FIG. 32).

As disclosed herein, the acyl donor substrate of PALs in the present invention is designed to preferably have an asparagine (Asn/N) at the P1 position, and glutamine (Gln/Q) at the P1′ position of the P1-P1′-P2′ tripeptide PAL recognition motif. Upon ligation with an acyl acceptor substrate, the acyl donor substrate releases a leaving group in which the exposed N-terminal glutamine is cyclized by QC, quenching the Gln N^{α}-amine in a lactam.

Without being bound to theory, it is believed that, upon cleavage of the Asn-Gln peptide bond, QC will cyclize the exposed Gln to form a pyroglutamyl residue (pGlu), thereby quenching its nucleophilic Na-amine in a lactam. Coupling PAL-mediated ligation with QC-catalyzed pGlu formation therefore advantageously overcomes the reversibility problem of the transpeptidative ligation reaction and provides an increased yield of ligated product.

To this end, provided in one aspect of the present disclosure is a method of enzymatic peptide ligation, said method comprising providing
i) a peptidyl asparaginyl ligase (PAL) and a glutaminyl cyclase (QC);
ii) a first peptide or protein having a P1-P1′-P2′ tripeptide PAL motif as an acyl donor, wherein P1 is Asn or Asp, P1′ is Gln or Glu and P2′ is a hydrophobic amino acid or a β-branched amino acid;
iii) a second peptide or protein which may be the same or different to the first peptide or protein, having a P1″-P2″ motif as an acyl acceptor at the N-terminus, wherein P1″ is any amino acid and P2″ is a hydrophobic amino acid or a B-branched amino acid;
iv) contacting the peptidyl asparaginyl ligase (PAL) and the glutaminyl cyclase (QC) with said first and second peptides/proteins;
wherein PAL cleaves the first peptide/protein after P1 in the tripeptide PAL motif and ligates said first peptide/protein to the P1″-P2″ motif of said second peptide/protein, and QC cyclizes P1′ in the released P1′-P2′ dipeptide motif to pyroglutamyl (pGlu).

It would be appreciated that PALs perform site-specific ligation reactions and require a minimal tripeptide recognition motif, P1-P1′-P2′, for ligation after P1, wherein P1 is typically Asn or Asp, and P1′ and P2′ may be any of the naturally occurring amino acids Gly, Ala, Val, Leu, He, Phe, Cys, Met, Pro, Thr, Ser, Glu, Gln, Asp, Asn, His, Lys, Arg, Tyr, and Trp.

For the purposes of the present invention, P1 is preferably Asn or Asp, P1′ is preferably Gln or Glu and P2′ is preferably a hydrophobic amino acid or a β-branched amino acid. In some embodiments, P1 is preferably Asn and P1′ is preferably Gln. It is known that Glu can act as a replacement for Gln at P1′ of the acyl donor (Seifert, F., et al., Biochemistry 48, 11831-11833 (2009)) and that Asp can act as a replacement for Asn at P1 of the acyl donor (Zhang, D., et al., (2021) Journal of the American Chemical Society 143 (23): 8704-8712). Accordingly in some embodiments, P1 may be Glu. In various embodiments, P1′ may be Asp.

In some embodiments, P2′ and/or P2″ may be a hydrophobic amino acid or a B-branched amino acid. Examples of a hydrophobic amino acid may include Gly, Ala, Val, Leu, Ile, Pro, Phe, Met, Tyr and Trp. Examples of a B-branched amino acid include Thr, Val, and Ile.

In some embodiments, P2′ may be selected from the group comprising Leu, Met, Phe, Tyr, Trp, Val, Ile and Thr. In various embodiments, P2″ may be selected from the group comprising Leu, Phe, Tyr, Trp, Val, Ile and Thr.

In some embodiments, the P1-P1′-P2′ tripeptide PAL motif of the acyl donor may be Asn-Gln-Leu.

It would be appreciated by a person skilled in the art that different PALs and variants thereof having the desired protein ligase activity may be suitable for the practice of the present invention. Accordingly in some embodiments, the PAL may be a butelase-1, butelase-2, VyPAL2, VyPAL3, OaAEP1b-C247A, HeAEP3, AtLEGy, VuPAL1, HaPAL1, OaAEP1b or a functional fragment or variant thereof.

In certain embodiments, the PAL may be selected from the group comprising butelase-1 comprising the amino acid sequence set forth in SEQ ID NO: 1, butelase-2 comprising the amino acid sequence set forth in SEQ ID NO: 2 or SEQ ID NO: 3, VyPAL2 comprising the amino acid sequence set forth in SEQ ID NO: 4, VyPAL3 comprising the amino acid sequence set forth in SEQ ID NO: 5, OaAEP1b-C247A comprising the amino acid sequence set forth in SEQ ID NO: 6, HeAEP3 comprising the amino acid sequence set forth in SEQ ID NO: 7, AtLEGγ comprising the amino acid sequence set forth in SEQ ID NO: 8, VuPAL1 comprising the amino acid sequence set forth in SEQ ID NO: 9, HaPAL1 comprising the amino acid sequence set forth in SEQ ID NO: 10, OaAEP1b comprising the amino acid sequence set forth in SEQ ID NO: 11 and a functional fragment or a variant thereof.

It is also envisaged that various QCs having the desired QC enzymatic activity may be suitable for use in the practice of the present invention. Accordingly in some embodiments, the QC may be a Human glutaminyl cyclase, a Mouse glutaminyl cyclase, a Drosophila glutaminyl cyclase, an Arabidopsis glutaminyl cyclase, a Conus glutaminyl cyclase, a Sistrurus glutaminyl cyclase, a Bacterial glutaminyl cyclase or a functional fragment or variant thereof.

In some embodiments, the QC may be selected from the group comprising Human glutaminyl cyclase comprising the amino acid sequence set forth in SEQ ID NO: 12, Mouse glutaminyl cyclase comprising the amino acid sequence set forth in SEQ ID NO: 13, Drosophila glutaminyl cyclase comprising the amino acid sequence set forth in SEQ ID NO: 14, Arabidopsis glutaminyl cyclase comprising the amino acid sequence set forth in SEQ ID NO: 15, Conus glutaminyl cyclase comprising the amino acid sequence set forth in SEQ ID NO: 16, Sistrurus glutaminyl cyclase comprising the amino acid sequence set forth in SEQ ID NO: 17, Bacterial glutaminyl cyclase comprising the amino acid sequence set forth in SEQ ID NO: 18 and a functional fragment or a variant thereof.

As those skilled in the art would appreciate, a protein/enzyme's function is directly related to its structure and sequence, and that there is a positive relationship between sequence identity and function similarity. In this regard, methods of determining a protein sequence identity are known in the art.

Accordingly, the sequences of the enzymes of the present disclosure may be sufficiently varied so long as the enzymes maintain their functionality and can exhibit the required activity (for example, the QC variant being able to catalyse the intramolecular cyclization of N-Terminal glutaminyl and glutamyl residues of peptides and proteins to form pyroglutamyl residue (pGlu)).

In some embodiments, the PAL may be a butelase-1 comprising the amino acid sequence with at least 85%, at least 90%, at least 95%, at least 96%, at least 97%, at least 98%, or at least 99% identity to the amino acid sequence set forth in SEQ ID NO: 1, a butelase-2 comprising the amino acid sequence with at least 85%, at least 90%, at least 95%, at least 96%, at least 97%, at least 98%, or at least 99% identity to the amino acid sequence set forth in set forth in SEQ ID NO: 2 or 3, a VyPAL2 comprising the amino acid sequence with at least 85%, at least 90%, at least 95%, at least 96%, at least 97%, at least 98%, or at least 99% identity to the amino acid sequence set forth in SEQ ID NO: 4, a VyPAL3 comprising the amino acid sequence with at least 85%, at least 90%, at least 95%, at least 96%, at least 97%, at least 98%, or at least 99% identity to the amino acid sequence set forth in SEQ ID NO: 5, a OaAEP1b-C247A comprising the amino acid sequence with at least 85%, at least 90%, at least 95%, at least 96%, at least 97%, at least 98%, or at least 99% identity to the amino acid sequence set forth in SEQ ID NO: 6, a HeAEP3 comprising the amino acid sequence with at least 85%, at least 90%, at least 95%, at least 96%, at least 97%, at least 98%, or at least 99% identity to the amino acid sequence set forth in SEQ ID NO: 7, a AtLEGy comprising the amino acid sequence with at least 85%, at least 90%, at least 95%, at least 96%, at least 97%, at least 98%, or at least 99% identity to the amino acid sequence set forth in SEQ ID NO: 8, a VuPAL1 comprising the amino acid sequence with at least 85%, at least 90%, at least 95%, at least 96%, at least 97%, at least 98%, or at least 99% identity to the amino acid sequence set forth in SEQ ID NO: 9, a HaPAL1 comprising the amino acid sequence with at least 85%, at least 90%, at least 95%, at least 96%, at least 97%, at least 98%, or at least 99% identity to the amino acid sequence set forth in SEQ ID NO: 10 or a OaAEP1b comprising the amino acid sequence with at least 85%, at least 90%, at least 95%, at least 96%, at least 97%, at least 98%, or at least 99% identity to the amino acid sequence set forth in SEQ ID NO: 11.

In some embodiments, the QC may be a Human glutaminyl cyclase comprising the amino acid sequence with at least 85%, at least 90%, at least 95%, at least 96%, at least 97%, at least 98%, or at least 99% identity to the amino acid sequence set forth SEQ ID NO: 12, a Mouse glutaminyl cyclase comprising the amino acid sequence with at least 85%, at least 90%, at least 95%, at least 96%, at least 97%, at least 98%, or at least 99% identity to the amino acid sequence set forth SEQ ID NO: 13, a Drosophila glutaminyl cyclase comprising the amino acid sequence with at least 85%, at least 90%, at least 95%, at least 96%, at least 97%, at least 98%, or at least 99% identity to the amino acid sequence set forth SEQ ID NO: 14, an Arabidopsis glutaminyl cyclase comprising the amino acid sequence with at least 85%, at least 90%, at least 95%, at least 96%, at least 97%, at least 98%, or at least 99% identity to the amino acid sequence set forth SEQ ID NO: 15, a Conus glutaminyl cyclase comprising the amino acid sequence with at least 85%, at least 90%, at least 95%, at least 96%, at least 97%, at least 98%, or at least 99% identity to the amino acid sequence set forth SEQ ID NO: 16, a Sistrurus glutaminyl cyclase comprising the amino acid sequence with at least 85%, at least 90%, at least 95%, at least 96%, at least 97%, at least 98%, or at least 99% identity to the amino acid sequence set forth SEQ ID NO: 17, or a Bacterial glutaminyl cyclase comprising the amino acid sequence with at least 85%, at least 90%, at least 95%, at least 96%, at least 97%, at least 98%, or at least 99% identity to the amino acid sequence set forth SEQ ID NO: 18.

In some embodiments, the second peptide or protein may comprise a spacer of at least one amino acid between the P1″-P2″ acyl acceptor and said second peptide or protein. As described herein, introducing a spacer between the P1″-P2″ acyl acceptor and said second peptide or protein may improve accessibility for the PAL to catalyse the ligation and consequently improve its yield, especially in cases where the second protein is large enough to hinder accessibility of PAL.

The rate of reaction of the method of the present disclosure may be controlled by varying the ratio of the enzyme to the substrate in question. In this regard, the inventors have found that the more enzyme used, the faster the reaction proceeded. In some embodiments, a small amount of enzyme (for example 0.005% eq of QC to the substrate and 1/10 eq to PAL) may be sufficient to carry out the invention. For some difficult protein substrates such as antibodies, a higher enzyme to substrate ratio may be required, such as 0.1:1:100 or 0.1:1:50 (QC: PAL: first peptide/protein). The ratio of enzyme to substrate to use is largely dependent on the substrate and the specific application, and may be easily determined using standard techniques known to those skilled in the art, or may be deduced by reference to the pertinent literature.

In some embodiments, the ratio of QC: PAL: first peptide or protein is in the range of 0.1:1:2000 to 1:1:50, respectively, and preferably in the range of 0.1:1:1000 to 0.1:1:50, respectively. In some embodiments, the ratio of QC: PAL: first peptide or protein is 0.1:1:20 respectively.

As described herein, the method of the present disclosure is suitable for protein-protein ligation and may be adapted for the preparation of, for example, antibody-drug conjugates, by appropriately selecting and modifying the acyl acceptor peptides and acyl donor substrates in accordance with the method of the present invention. As such, it is also within the scope of the present invention that the method of the present disclosure may be adapted for the effective ligation of monoclonal antibodies and other proteins with a broad range of linker-payload drug compounds (for example, with the linker-payload drug compounds as the acyl acceptor substrates). Accordingly, modified peptides for use in the present invention may be prepared using standard techniques known to those skilled in the art of synthetic organic chemistry, or may be deduced by reference to the pertinent literature.

In some embodiments, the first and second peptides or proteins to be ligated in accordance with the present application may be further modified to comprise a labelling component. A labelling component may be any molecules such as, without limitation, an affinity tag, a detectable label, a therapeutic agent, a scaffold molecule, an epitope-binding peptide, ubiquitin molecule, biotin molecule, His₆ tag, Green fluorescent protein (GFP), an epitope-binding peptide, and affibodies such as Z_{EGFR}, Z_{EGFR}-Fc and DARPin.

In some embodiments, the said first and second peptides or proteins may be the same and may form a dimer upon ligation.

It would be appreciated that the key components of antibody-drug conjugates may include an antibody, a linker and a payload. Accordingly, in some embodiments, one of said first and second peptides or proteins may be an epitope-binding peptide or protein and the other peptide or protein may comprise a payload. In some embodiments, the payload may comprise a payload-releasing linkage.

In some embodiments, the payload may an imaging agent or a therapeutic agent. In particular, the imaging agent may be a radiolabel chelator or an optical label.

In some embodiments, the radiolabel chelator may be selected from the group comprising 1,4,7-triazacyclononanetriacetic acid (NOTA), 1,4,7, 10-tetraazacyclododecane-1,4,7,10-tetraacetic acid (DOTA) and 1,4,7-triazacyclononane-1-glutaric acid-4,7-diacetic acid (NODAGA) and/or the optical label is HRP or GFP or the like. In some embodiments, the therapeutic agent may be Monomethyl auristatin E (MMAE) or radiolabelled DOTA.

In various embodiments, the epitope-binding peptide or protein may be selected from the group comprising an antibody or functional fragment thereof, an affibody such as Z_{EGFR} or Z_{EGFR}-FC, and DARPin.

In certain embodiments, the antibody or functional fragment thereof is selected from the group comprising minibody, diabody, scFv, nanobody and F(ab′)₂.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Materials and General Methods

All the solvents and reagents were purchased from commercial suppliers and used without further purification. Human Glutaminyl Cyclase (QC) was purchase from Abcam (ab206806), aliquoted and stored at −80° C.

Peptides were synthesized following standard Fmoc solid phase synthesis protocols. Synthesized peptides were purified using semi-preparative RP-HPLC. Semi-preparative RP-HPLC was performed using a Shimadzu HPLC system equipped with a Phenomenex-C18 RP column (10×250 mm, 5 μm) with a flow rate of 2.5 mL/min, eluting using a gradient of buffer B (90% acetonitrile, 10% H₂O, 0.045% TFA) in buffer A (H₂O, 0.045% TFA). All the synthesized compounds were stored at 4° C. or −20° C.

Proteins were generated using recombinant DNA methods. For protein purification, Immobilized Metal Affinity Chromatography (IMAC), Protein A affinity chromatography and Size-Exclusion chromatography (SEC) were used. SEC was performed on the ÄKTA FPLC UPC-900 using HiLoad™ 16/600 Superdex™ 200 pg column. Protein A and NiNTA affinity chromatography was conducted on ÄKTAstart using HiTrap 5 ml MabSelect™ column or His Trap HP 5 ml column, respectively.

For analysis, mass spectra for peptides were obtained using a Bruker Ultraflex Extreme Matrix Assisted Laser Desorption/Ionization (MALDI) Tandem TOF or electrospray ionization (ESI) mass spectroscopy (Thermo Fisher LTQ XL). Data from MALDI was analysed using Data Explorer software, and data from ESI was analysed using Thermo Xcalibur Qual Browser and Magtran software. The deconvolution of protein mass spectra was done using MagTran. Analytical reverse-phase HPLC (RP-HPLC) was performed on a Shimadzu HPLC system equipped with a Phenomenex-C18 RP column (4.6×150 mm, 55 μm, 100 Å) or a Phenomenex Jupiter-C4 column (4.6×150 mm, 3.6 μm, 200 Å) with a flow rate of 1.0 mL per minute, eluting with a gradient of buffer B (90% ACN, 10% H₂O, 0.045% TFA) in buffer A (H₂O, 0.045% TFA).

### Solid Phase Peptide Synthesis (SPPS)

All the peptides were synthesized as C-terminal amides using Rink amide MBHA resin by standard Fmoc chemistry using Liberty Blue Peptide Synthesizer or using 2-Chlorotrityl chloride resin. For 5(6)-carboxyfluorescein and biotin coupling to Lys (MTT) sidechain, the MTT protecting group was first removed using TFA/TIS/DCM (2.5%/2.5%/95%), followed by 5(6)-carboxyfluorescein (or biotin) coupling to the Lys sidechain amine using 2.5 eq 5(6)-carboxyfluorescein (or biotin), 2.5 eq Oxyma, 2.5 eq DIC in NMP for 3 h. For peptide cleavage from resin and deprotection of sidechain protecting groups at the end of SPPS, the peptidyl-resin was treated with a cocktail of TFA/H₂O/TIS (95%/2.5%/2.5%) for 1-3 hours. The cleavage solution was separated from the resin by filtration and the cleaved peptide was precipitated in the cold Et₂O. The crude product was isolated by centrifugation and purified by RP-HPLC. The peptide fractions after HPLC purification were lyophilized to afford the peptide in powder form.

### Synthesis of GIGKVA-PABC-MMAE (Compound 4)

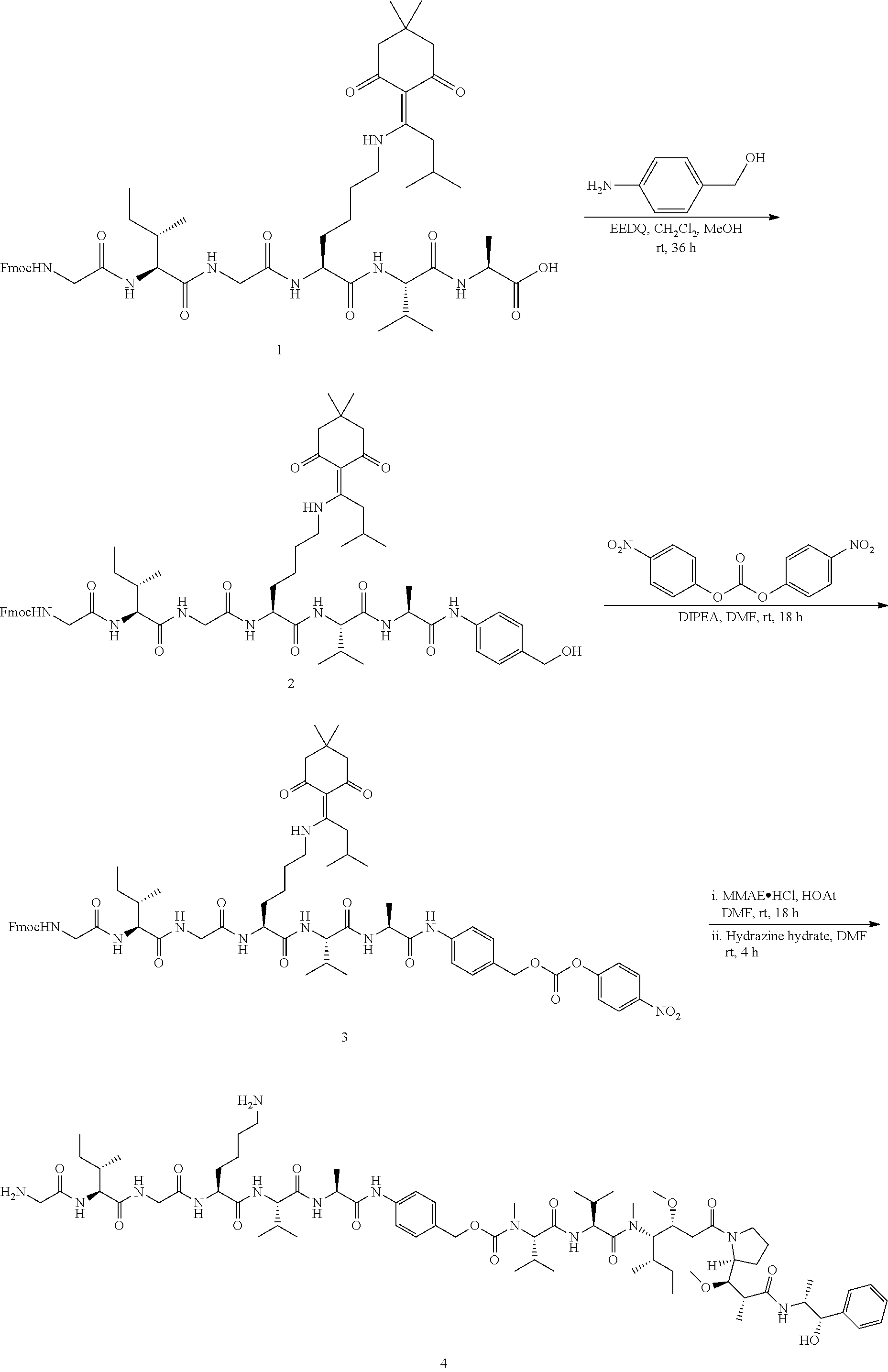

Compound 1 was synthesized using standard SPPS chemistry.

FmocGIGK (ivDde) VA-PAB-OH (2): To a suspension of compound 1 (110 mg, 0.113 mmol) in MeOH (2.5 mL) and DCM (5.0 mL) were added EEDQ (84 mg, 0.34 mmol) and 4-aminobenzyl alcohol (27.8 mg, 0.226 mmol). The mixture was stirred under the dark at room temperature for 36 h. After evaporation of the solvent, the residue was subjected to column chromatography (2-6% MeOH in DCM) to yield compound 2 (70 mg, 58%) as off-white solid.

FmocGIGK(ivDde)VA-PAB-PNP carbonate 3 was prepared by adding DIPEA (60 μL, 0.33 mmol) and bis (p-nitrophenyl) carbonate (100 mg, 0.33 mmol) to a solution of compound 2 (120 mg, 0.11 mmol) in anhydrous DMF, and the mixture under N₂ atmosphere was stirred at room temperature for 18 h. After solvent removal by rotary evaporation, the residue was subjected to column chromatography (2-4% MeOH in DCM) to yield the PNP-carbonate 3 (110mg, 79%) as off-white solid.

MMAE⋅HCl (66 mg, 0.088 mmol) was added to a solution of compound 3 (100 mg, 0.081 mmol), HOAt (5.5 mg, 0.04 mmol) and DIPEA (0.07 mL, 0.405 mmol) in anhydrous DMF. The resulting reaction mixture was stirred at room temperature for 18 h. Hydrazine hydrate (0.5mL) was then added, and the mixture was stirred for 4 h. After removing solvent by rotary evaporation, the residue was subjected to reverse-phase HPLC purification (Buffer A: 0.045% TFA in H₂O, Buffer B: 0.045% TFA in 90% acetonitrile, 10% H₂O). The fractions containing the product were pooled and freeze dried to afford compound 4 as off-white powder. MS (ESI): m/z [M+H]⁺ calc. 1392.9, found 1393.0.

### Synthesis of GIGGGK[Fe(DOTA)] (Compound 6)

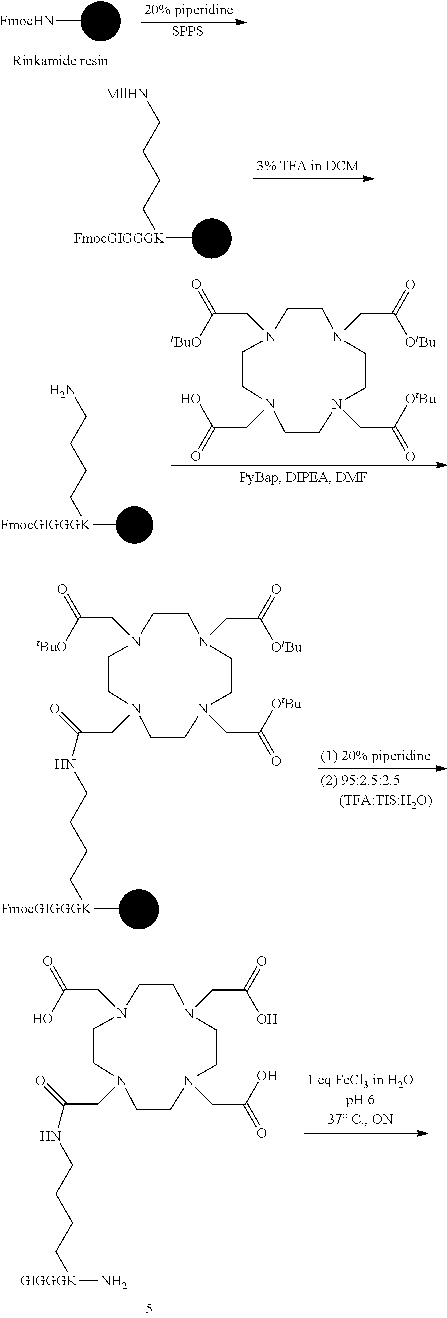

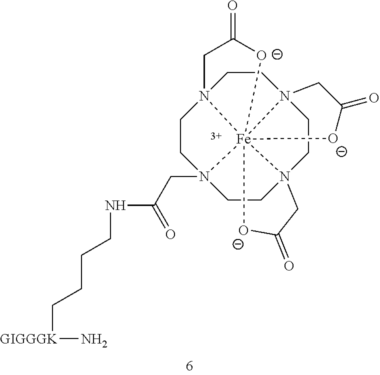

Compound 5 was synthesized using standard SPPS chemistry. 1 eq of compound 5 (10 mM) was mixed with 1 eq of FeCl₃ in water and the pH of the solution was adjusted to pH 6 with 2 M NaOH. The mixture was left at 37° C. for overnight to afford GIGGGK[Fe(DOTA)] which was used without purification. MS (ESI): m/z [M+H]⁺ calc. 926.6, found 926.5.

### OaAEP1_{b}-C247A Expression, Purification and Activation

OaAEP1_{b}-C247A was cloned into vector pET28a (Genscript) and expressed using T7 SHuffle E. coli. Pro-OaAEP1_{b}-C247A was activated at pH 4 in acetic buffer (0.1 M NaCl, 0.5 mM TCEP) for 2 h at 37° C. After activation, the activated enzyme was purified by size-exclusion chromatography (SEC) at pH 7 (20 mM PBS, 0.1 M NaCl). Purified enzyme was stored at −80° C. in 5% glycerol, pH 7 (20 mM PBS, 0.5 mM TCEP).

### VyPAL2 Expression, Purification and Activation

VyPAL2 was expressed using sf9 insect cells. 100 mL of the viral vector containing VyPAL2 gene was used to infect sf9 cells at cell density of 2.5×10⁶ cells/mL. MOI for infection was set between 1-10 for protein expression. The culture was incubated in a 27° C. shaker for 3 days (72 hours) at 135 rpm. Protein purification was performed in three steps: Immobilized Metal Affinity Chromatography (IMAC), lon-Exchange Chromatography (IEX), and Size-Exclusion chromatography (SEC). Pro-VyPAL2 was activated at pH 4.5 in 50 mM sodium citrate buffer (0.1 M NaCl, 1 mM DTT, 0.5 mM LS) for 2-3 h at 37° C. After activation, the activated enzyme was purified by SEC at pH 6.5 (20 mM PBS, 0.1 M NaCl, 1 mM DTT). Purified enzyme was stored at −80° C. in 5% glycerol, pH 7 (20 mM PBS, 0.5 mM TCEP).

### DARPin9 26, GFP, Ubiquitin, Z_{EGFR} and Z_{EGFR}-Fc Expression and Purification

All protein genes were cloned in pET28a, pET3a, pTxB1 or pETDuet (Genscript), and expressed in E. coli (DE3) or T7 SHuffle. The expressions (except for GI-ubi) were induced with 0.1-0.4 mM IPTG after the OD₆₀₀ of bacteria reached 0.4-0.6 in Luria Bertani broth (Kana or Amp) at 37° C. or 30° C. After induction, the cells were incubated at 16° C. for 18 h. The cells were harvested by centrifugation (5000×g, 10 min) and resuspended in lysis buffer (50 mM PBS, 0.1 M NaCl, 10 mM imidazole, 0.01% 100× triton, pH7.5). The solution mixture was lysed using ultrasonicator probe (Vibra cell™) with alternative cycles of 3 s pulse after every 8 s interval for 15-30 min on ice. The protein solution was then centrifuged at 15000×g (20 min) at 4° C., filtered using 0.2 μm membrane, and bound to NiNTA beads or protein A beads for 1 h at 4° C. The Ni beads were washed with 20 mM imidazole, 0.1 M NaCl, 20 mM PBS buffer (pH 7.5), then protein was eluted using 500 mM imidazole, 0.1 M NaCl, 20 mM PBS buffer (pH 7.5). The protein A beads were washed with 20 mM PBS (pH7.5), then the protein was eluted using 30 mM citrate buffer (pH 3.5). For GI-ubi, it was expressed as a C-terminal intein fusion protein in E.coli (DE3), the protein solution was bound to chitin beads and the GI-ubi was cleaved from bounded intein by incubating in 50 mM DTT, 20 mM PBS (pH 8) overnight, at RT. All the proteins were exchanged into 20 mM PBS (pH 7) and stored at 4° C. for short term and −20° C. for long term.

### Ligation of Model Peptides

Enzyme-meditated ligation reactions were performed in 20 mM PBS buffer (pH 6.5 or pH 7) at 37° C. for various time courses with or without QC. The ratio of QC to ligase to substrate (NQL peptide) is 0.1:1:2000. The reactions were quenched by 10% TFA and monitored by analytical RP-HPLC. The ligated products were characterized by MALDI-MS or ESI-MS.

### Protein N or C Terminal Labelling

The ligation reactions were conducted at pH 7 under 37° C. for various time courses with or without QC. The ratio of QC/ligase/protein substrate is 0.1/1/1000. The reactions were quenched by 6 M Guanidine-HCl (pH 3) and the reaction was monitored by analytical RP-HPLC. The ligated products were characterized by ESI-MS.

### Protein-Protein Ligation

The ligation reactions were conducted at pH 7 under 37° C. for various time courses with or without QC. The ratio of Qc/ligase/protein substrate is 0.1/1/1000 (500). The reactions were quenched by 6 M Guanidine-HCl (pH 3) and the completion reaction was monitored by analytical RP-HPLC. The ligated products were characterized by ESI-MS. The ligation reaction of Z_{EGFR}-FC-NQL and GI-GGGSGGGS-GFP was analysed by SDS-PAGE under reducing or non-reducing conditions (reducing condition: 50 mM DTT, pH 8.8 for 20 min).

### Purification of Ligated Proteins

The ligated Z_{EGFR}-Fc-GFP protein was purified by Size-Exclusion chromatography (SEC) at pH 7 (20 mM PBS, 0.1 M NaCl). The purified protein was stored at −20° C. The ligated Z_{EGFR}-MMAE was purified by Immobilized Metal Affinity Chromatography (IMAC) and stored in pH 7 buffer (20 mM PBS, 0.1 M NaCl).

### Flow Cytometry Assay

To study binding capacities of Z_{EGFR}-Fc-GFP, A-431 (ATCC, USA) and MCF-7 (ATCC, US) live cells were washed three times with PBS (HyClone, USA), trypsinized by 0.05% Trypsin-EDTA (Gibco, USA), and then resuspended in chilled DMEM (Gibco, USA) with 10% FBS (Gibco, USA). One million cells of each cell line were then incubated with Z_{EGFR}-Fc-GFP (100 nM) and GFP (100 nM) on ice for 30 min. After incubation, the cells were washed with chilled PBS for three times and analyzed by the Fortessa X-20 flow cytometer (BD, USA). The cytometer was set to record 10,000 events per sample, to excite the fluorophore with 488 nm laser, and to collect emitting fluorescent signals in 530/30 nm. The generated raw data were analyzed by Flowjo™ 10 (BD, USA).

### Live Cell Confocal Imaging

To visualize Z_{EGFR}-Fc-GFP binding activities, A431 and MCF-7 cells were seeded on an 8-well chamber slide (ibidi, USA) and incubated at 37° C. under 5% CO₂ overnight. The cells were stained with 2 μM PKH26 red-fluorescent dye (Sigma, USA) for 10 min at 37° C. The stained cells were then incubated with Z_{EGFR}-Fc-GFP (100nM) and GFP (100nM) on ice for 30 min. After incubation, the cells were washed with chilled PBS for three times and fixed with cold 4% formaldehyde for 15 min. The fixed cells were imaged by the LSM 980 confocal microscope (Zeiss, Germany). Microscopic key settings are as follows: 1) excitation laser wavelengths: 488 nm and 561 nm; 2) emission fillers: 507-552 nm and 575-620 nm; 3) imaging mode: Z-stack. The 3D Z-stack images were processed into 2D images by the technique MIP (maximum intensity projection) using the Zen software (Zeiss, Germany).

### Cell Viability Assay

To test the cytotoxicity of Z_{EGFR}-MMAE, ˜5000 A431 and MCF-7 cells were seeded separately on a 96-well plate and incubated at 37° C. under 5% CO₂ overnight. Z_{EGFR}-MMAE was added to wells at different concentrations and incubated at 37° C. under 5% CO₂ for 3 days. Then 0.5 mg/ml of MTT was added and incubation continued at 37° C. for 1 h. The viability of cells was determined based on the absorbance at 570 nm.

### Peptide and Protein Sequences and Mass Spectrometry Data

TABLE 1
Peptides used in the study

| | |
|---|---|
| 01 | 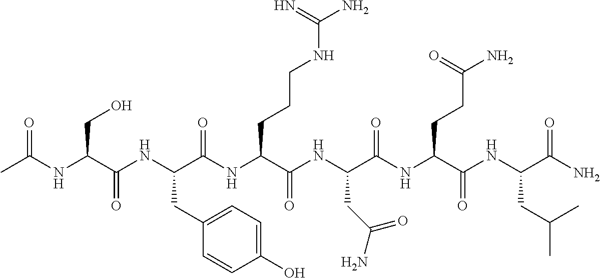 [SMILES: CC(=O)N[C@@H](CO)C(=O)N[C@@H](Cc1ccc(O)cc1)C(=O)N[C@@H](CCCNC(=N)N)C(=O)N[C@@H](CC(N)=O)C(=O)N[C@@H](CCC(N)=O)C(=O)N[C@@H](CC(C)C)C(N)=O] SEQ ID NO. 27: Ac-SYRNQL (m/z [M + H]⁺ calc. 821.4, obvs. 821.5) |
| 02 | 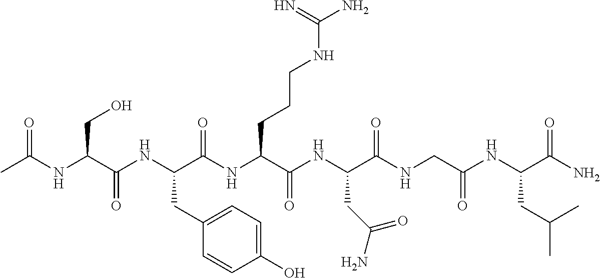 [SMILES: CC(=O)N[C@@H](CO)C(=O)N[C@@H](Cc1ccc(O)cc1)C(=O)N[C@@H](CCCNC(=N)N)C(=O)N[C@@H](CC(N)=O)C(=O)NCC(=O)N[C@@H](CC(C)C)C(N)=O] SEQ ID NO. 28: Ac-SYRNGL (m/z [M + H]⁺ calc. 750.4, obvs. 750.5) |
| 03 | 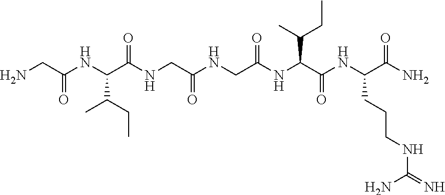 [SMILES: CCC(C)[C@H](NC(=O)CN)C(=O)NCC(=O)NCC(=O)N[C@H](C(=O)N[C@@H](CCCNC(=N)N)C(N)=O)C(C)CC] SEQ ID NO. 29: GIGGIR (m/z [M + H]⁺ calc. 571.4, obvs. 571.5) |
| 04 | 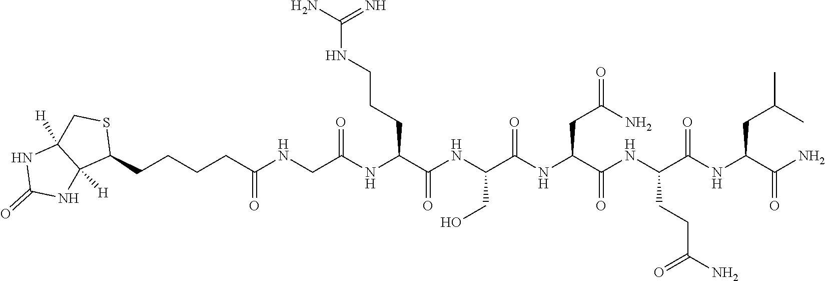 [SMILES: CC(C)C[C@H](NC(=O)[C@H](CCC(N)=O)NC(=O)[C@H](CC(N)=O)NC(=O)[C@H](CO)NC(=O)[C@H](CCCNC(=N)N)NC(=O)CNC(=O)CCCC[C@@H]1SC[C@@H]2NC(=O)N[C@@H]21)C(N)=O] SEQ ID NO. 30: Biotin-GRSNQL (m/z [M + H]⁺ calc. 899.4, obvs.899.8) |
| 05 | 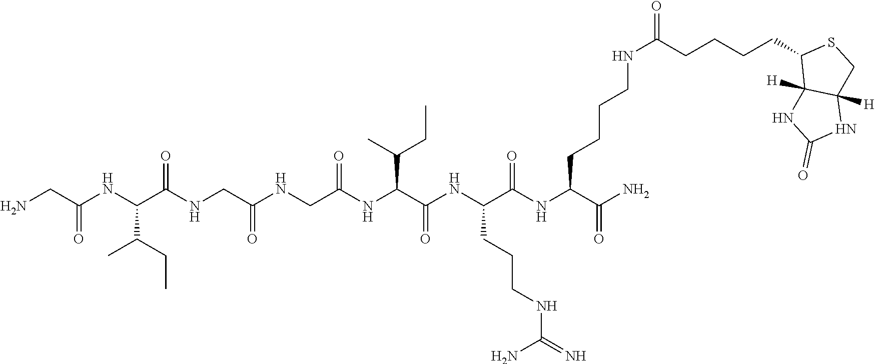 [SMILES: CCC(C)[C@H](NC(=O)CN)C(=O)NCC(=O)NCC(=O)N[C@H](C(=O)N[C@@H](CCCNC(=N)N)C(=O)N[C@@H](CCCCNC(=O)CCCC[C@@H]1SC[C@@H]2NC(=O)N[C@@H]21)C(N)=O)C(C)CC] SEQ ID NO. 31: GIGGIRK(biotin) (m/z [M + H]⁺ calc. 925.5, obvs. 925.7) |
| 06 | R = Leu, Ile, Val or Phe 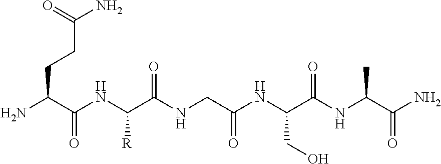 SEQ ID NO. 32: QLGSA (m/z [M + H]⁺ calc. 474.3, obvs. 474.5); SEQ ID NO. 33: QFGSA (m/z [M + H]⁺ calc. 508.2, obvs. 508.3); SEQ ID NO. 34: QIGSA (m/z [M + H]⁺ calc. 474.3, obvs. 474.3); SEQ ID NO. 35: QVGSA (m/z [M + H]⁺ calc. 460.3, obvs. 460.5) |
| 07 | 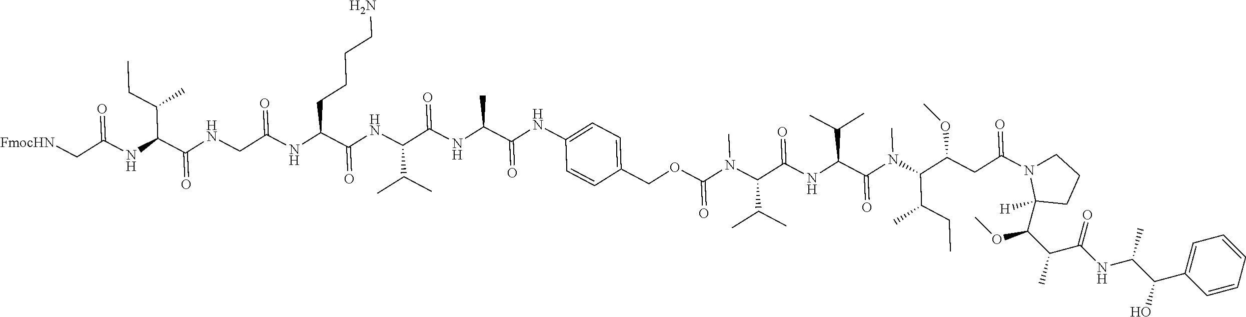 SEQ ID NO. 36: GIGKVA-PABC-MMAE (m/z [M + H]⁺ calc. 1392.9, obvs. 1393.0) |
| 08 | 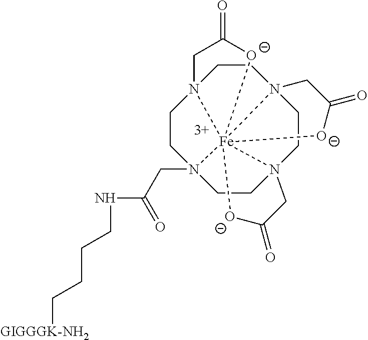 SEQ ID NO. 37: GIGGGK(DOTA-Fe³⁺) (m/z [M + H]⁺ calc. 926.4, obvs. 926.6) |
| 09 | 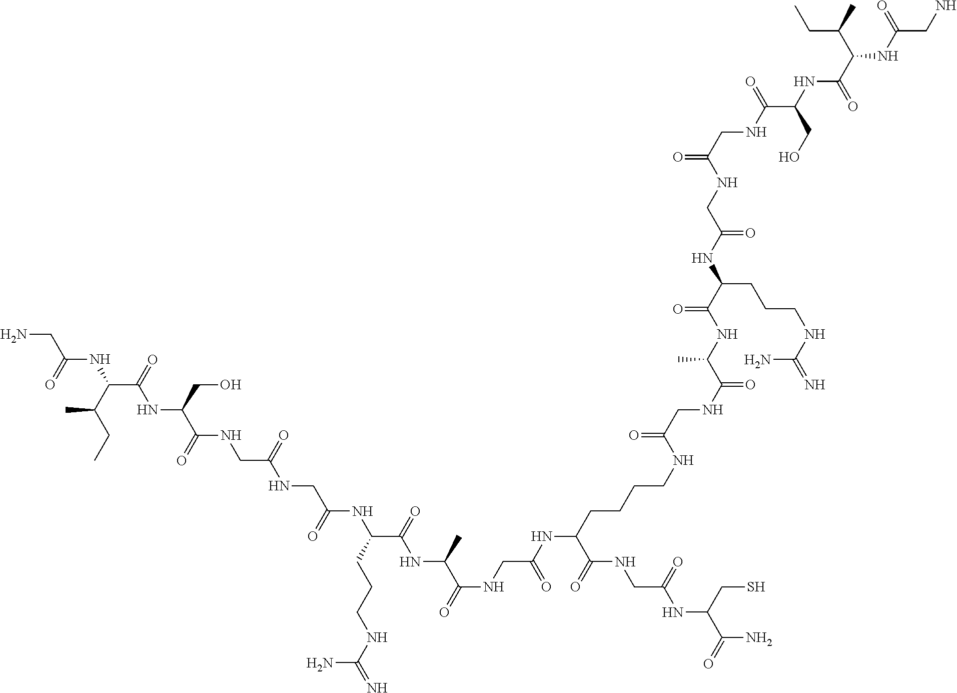 [SMILES: CC[C@@H](C)[C@H](NC(=O)CN)C(=O)N[C@@H](CO)C(=O)NCC(=O)NCC(=O)N[C@@H](CCCNC(=N)N)C(=O)N[C@@H](C)C(=O)NCC(=O)NCCCCC(NC(=O)CNC(=O)[C@H](C)NC(=O)[C@H](CCCNC(=N)N)NC(=O)CNC(=O)CNC(=O)[C@H](CO)NC(=O)[C@@H](NC(=O)CN)[C@H](C)CC)C(=O)NCC(=O)NC(CS)C(N)=O] (GISGGRAG)₂KGC (calc. 1615.8 [M + H]⁺; obsv. 808.6 [M + 2H]²⁺) SEQ ID NO. 38: GISGGRAGKGC SEQ ID NO. 39: GISGGRAG |

TABLE 2
Peptide Asparaginyl Ligases/Asparaginyl Endopeptidases
and their amino acid sequences

| | |
|---|---|
| Butelase-1 (Clitoria ternatea) | MKNPLAILFL IATVVAVVSG IRDDFLRLPS |
| SEQ ID NO: 1 | QASKFFQADD NVEGTRWAVL VAGSKGYVNY |
| Length: 482 amino acids | RHQADVCHAY QILKKGGLKD ENIIVFMYDD |
| | IAYNESNPHP GVIINHPYGS DVYKGVPKDY |
| | VGEDINPPNF YAVLLANKSA LTGTGSGKVL |
| | DSGPNDHVFI YYTDHGGAGV LGMPSKPYIA |
| | ASDLNDVLKK KHASGTYKSI VFYVESCESG |
| | SMFDGLLPED HNIYVMGASD TGESSWVTYC |
| | PLQHPSPPPE YDVCVGDLFS VAWLEDCDVH |
| | NLQTETFQQQ YEVVKNKTIV ALIEDGTHVV |
| | QYGDVGLSKQ TLFVYMGTDP ANDNNTFTDK |
| | NSLGTPRKAV SQRDADLIHY WEKYRRAPEG |
| | SSRKAEAKKQ LREVMAHRMH IDNSVKHIGK |
| | LLFGIEKGHK MLNNVRPAGL PVVDDWDCFK |
| | TLIRTFETCH GSLSEYGMKH MRSFANLCNA |
| | GIRKEQMAEA SAQACVSIPD NPWSSLHAGF |
| | SV |
| Butelase-2 (Clitoria ternatea) | MGHHHHHHSS GVDLGTENLY FQSMARLNPQ |
| G252V G182A | KEWDSVIRLP TEPVDADTDE VGTRWAVLVA |
| SEQ ID NO: 2 | GSNGYENYRH QADVCHAYQL LIKGGLKEEN |
| Length: 480 amino acids | IVVFMYDDIA WHELNPRPGV IINNPRGEDV |
| | YAGVPKDYTG EDVTAENLFA VILGDRSKVK |
| | GGSGKVINSK PEDRIFIFYS DHGAPGVLGM |
| | PNEQILYAMD FIDVLKKKHA SGGYREMVIY |
| | VEACESGSLF EGIMPKDLNV DHGAPGVLGM |
| | NSWVTYCPGT EPSPPPEYTT SGGYREMVIY |
| | MEDSESHNLR RETVNQQYRS FVTTASNAQE |
| | YAMGSHVMQY GDTNITAEKL YLFQGFDPAT |
| | VNLPPHNGRI EAKMEVVHQR DAELLEMWQM |
| | YQRSNHLLGK KTHILKQIAE TVKHRNHLDG |
| | SVELIGVLLY GPGKGSPVLQ SVRDPGLPLV |
| | DNWACLKSMV RVFESHCGSL TQYGMKHMRA |
| | FANICNSGVS ESSMEEACMV ACGGHDAGHL |
| Butelase-2 (Clitoria ternatea) | MGHHHHHHSS GVDLGTENLY FQSMARLNPQ |
| G252V P183A | KEWDSVIRLP TEPVDADTDE VGTRWAVLVA |
| SEQ ID NO: 3 | GSNGYENYRH QADVCHAYQL LIKGGLKEEN |
| Length: 480amino acids | IVVFMYDDIA WHELNPRPGV IINNPRGEDV |
| | YAGVPKDYTG EDVTAENLFA VILGDRSKVK |
| | GGSGKVINSK PEDRIFIFYS DHGGAGVLGM |
| | PNEQILYAMD FIDVLKKKHA SGGYREMVIY |
| | VEACESGSLF EGIMPKDLNV FVTTASNAQE |
| | NSWVTYCPGT EPSPPPEYTT CLGDLYSVAW |
| | MEDSESHNLR RETVNQQYRS VKERTSNFKD |
| | YAMGSHVMQY GDTNITAEKL YLFQGFDPAT |
| | NLPPPHNGRI EAKMEVVHQR DAELLFMWQM |
| | YQRSNHLLGK KTHILKQIAE TVKHRNHLDG |
| | SVELIGVLLY GPGKGSPVLQ SVRDPGLPLV |
| | DNWACLKSMV RVFESHCGSL TQYGMKHMRA |
| | FANICNSGVS ESSMEEACMV ACGGHDAGHL |
| VyPAL2 (Viola yedoensis) | MQLFAAGVIL FFLLALSGTI AGGLDVDSLQ |
| SEQ ID NO: 4 | LPSEAAKFFH NDNSTNDDDS IGTRWAVLIA |
| Length: 483 amino acids | GSKGYHNYRH QADVCHMYQI LRKGGVKDEN |
| | IIVFMYDDIA YNESNPFPGI IINKPGGENV |
| | YKGVPKDYTG EDINNVNFLA AILGNKSAII |
| | GGSGKVLDTS PNDHIFIYYA DHGAPGKIGM |
| | PSKPYLYADD LVDTLKQKAA TGTYKSMVFY |
| | VEACNAGSMF EGLLPEGTNI YAMAASNSTE |
| | GSWITYCPGT PDFPPEFDVC LGDLWSITFL |
| | EDCDAHNLRT ETVHQQFELV KKKIAYASTV |
| | SQYGDIPISK DSLSVYMGTD PANDNRTFVD |
| | ENSLRPPLKV IHQHDADLYH IWCKYNMAPE |
| | GSSKKIEAQK QLLELMSHRA HVDNSITLIG |
| | KLLFGVNKAS KVLNTVRPVG QPLVDDWQCL |
| | KAMIRTFETH CGSLSEYGMK HTLSFANMCN |
| | AGIQKEQLAE AAAQACVTFP SNPYSSLAEG |
| | FSA |
| VyPAL2 (Viola yedoensis) | MQLFAAGVIL FFLLALSGTI AGGLDVDSLQ |
| SEQ ID NO: 5 | LPSEAAKFFH NDNSTNDDSS AGTKWAVLIA |
| Length: 449 amino acids | GSKGYQNYRH QADVCHAYQI LRRGGVKDEN |
| | IIVFMYDDIA YDIRNPYPGT ITNSPDKKDV |
| | YKGVPDKYTG EDVNVQNFLA VILGNKTALT |
| | GGSGKVLDTR PNDHIFIYYT DHGYAGVLGM |
| | PTQPYLYAND LIDTLKKKHA SGTYESLVFY |
| | VEACESASIF EGLLPDGLNI YVSTAAKAGE |
| | GSWVVYCPTQ QPPVPAEYGT CVGDLYSVTW |
| | MEDCDLYNLR TQTLHQQYEM VKKKIAYAST |
| | VSQFGDLTIT KDSLFEYMGT DPANEKHHYE |
| | DQENSLRPHV DAVHQREADL YHFWDKYQKA |
| | SEGSRNKVAA RKQLVEVMLH RMHVDDSIES |
| | IAKLLFGSDA KASEMMNTIR PPGQPLVSDW |
| | DCLKTMVRTF ETHCGSLSEY GMKYTRFLA |
| OaAEP1b-C247A (oldenlandia affinis) | MGMAHHHHHH MQIFVKTLTG KTITLEVEPS |
| SEQ ID NO: 6 | DTIENVKAKI QDKEGIPPDQ QRLIFAGKQL |
| Length: 537 amino acids | EDGRTLSDYN IQKESTLHLV LRLRGGARDG |
| | DYLHLPSEVS RFFRPQETND DHGEDSVGTR |
| | WAVLIAGSKG YANYRHQAGV CHAYQILKRG |
| | GLKDENIVVF MYDDIAYNES NPRPGVIINS |
| | PHGSDVYAGV PKDYTGEEVN AKNFLAAILG |
| | NKSAIKGGSG KVVDSGPNDH IFIYYTDHGA |
| | AGVIGMPSKP YLYADELNDA LKKKHASGTY |
| | KSLVFYLEAC ESGSMFEGIL PEDLNIYALT |
| | STNTTESSWA YYCPAQENPP PPEYNVCLGD |
| | LFSVAWLEDS DVQNSWYETL NQQYHHVDKR |
| | ISHASHATQY GNLKLGEEGL FVYMGSNPAN |
| | DNYTSLDGNA LTPSSIVVNQ RDADLLHLWE |
| | KFRKAPEGSA RKEEAQTQIF KAMSHRVHID |
| | SSIKLIGKLL FGIEKCTEIL NAVRPAGQPL |
| | VDDWACLRSL VGTFETHCGS LSEYGMRHTR |
| | TIANICNAGI SEEQMAEAAS QACASIP |
| HeAEP3 | MKLLVPGVLL LFLLALSGIA AGRPDDFLRL |
| (Afrohybanthus enneaspermus) | PSEAAKSFLH NDDDSVGTRW AVLIAGSKGW |
| SEQ ID NO: 7 | QNYRHQADVC HAYQILKKGG LKDENIVVFM |
| Length: 481 amino acids | YDDIAYNESN PRPGIVINKP KGEDVYKGVP |
| | KDYTGENVNA VNFLAVLLAN RSALTGGSGK |
| | VLDSGPNDRI FIYYTDHGAP VTIGMPSKPY |
| | LVAKDLVDTL KKKHAAGTYK SMVFYIESCE |
| | SGSMFDGLLP EDANIYGMTA TNSTEGSWVT |
| | YCPGQTDDYP EDDEYDVCFG DLWSVAWLED |
| | CDAHNLRTET LDQQYEVVKK KIEYAHIPAQ |
| | YGNVSLAKDS LFVYMGTDPA NDNKTFVEEN |
| | TLRRPLKAVH SRDADLLHFW HKYHKAPEGT |
| | SRKIDAQKQL VEVLSHRTHV DNSIKLVGEL |
| | LFGVGKASEV LNTIRPAGQP LVDDWDCLKT |
| | MVRTFETHCG SLSEYGMKHM RSFANMCNAG |
| | VQKEQMAVAA GQACVTFPSN PWSSLDEGFS |
| | V |
| AtLEGγ (Arabidopsis thaliana) | SLEHHHHHHE NLYFQGVGTR WAVLVAGSSG |
| SEQ ID NO: 8 | YGNYRHQADV CHAYQILRKG GLKEENIVVL |
| Length: 455 amino acids | MYDDIANHPL NPRPGTLINH PDGDDVYAGV |
| | PKDYTGSSVT AANFYAVLLG DQKAVKGGSG |
| | KVIASKPNDH IFVYYAXHGG PGLVGMPNTP |
| | HIYAADFIET LKKKHASGTY KEMVIYVEAA |
| | ESGSIFEGIM PKDLNIYVTT ASNAQESSYG |
| | TYCPGMNPSP PSEYITCLGD LYSVAWMEDS |
| | ETHNLKKETI KQQYHTVKMR TSNYNTYSGG |
| | SHVMEYGNNS IKSEKLYLYQ GFDPATVNLP |
| | LNELPVKSKI GVVNQRDADL LFLWHMYRTS |
| | EDGSRKKDDT LKELTETTRH RKHLDSAVEL |
| | IATILFGPTM NVLNLVREPG LPLVDDWECL |
| | KSMVRVFEEH CGSLTQYGMK HMRAFANVCN |
| | NGVSKELMEE ASTAACGGYS EARYTVHPSI |
| | LGYSA |
| VuPAL1 (Viola uliginosa) | MKLLAAGVIL VSLLALSGTV AGGLDVDPLR |
| SEQ ID NO: 9 | LPSEAAKFFH NDNSTNDDDS IGTRWAVLIA |
| Length: 484 amino acids | GSKDYHNYRH QADVCHMYQI LRKGGVKDEN |
| | IIVFMYDDIA YNESNPHPGI IINKPGGEDV |
| | YKGVPKDTYG EDVNNINFLA AILGNKSAII |
| | GGSGKVLDTS PNDHIFIYYT DHGAPGKIGM |
| | PSKPYLYADD LVDTLKQKAA TGTYKSMVFY |
| | VEACNAGSMF EGLLPEGTNI YAMAASNSTE |
| | GSWITYCPGA TPDFPPEYDI CLGDLWSITF |
| | LEDCDAHNLR TETVHQQFEL VKKNIAYAST |
| | VSQYGDIPIS KDSLSVYMGT DPANDNRTFV |
| | DENSLKPPLK VIHQRDADLY HLWYKYNKAP |
| | EGSSKKEIAQ KQLLELMSHR AHVDNSITLI |
| | GKLLFGVDKA SKVLNTVRPV GQPLVDDWQC |
| | LKAMIRTFET HCGSLSEYGM KHTLSFANMC |
| | NAGIQKEQLA EAAAQACVTF PSNSYSSSLE |
| | GFSA |
| HaPAL1 (Helianthus annuus) | MACFSYRLIC LLLVLMMVMA LPNGAAAARR |
| SEQ ID NO: 10 | GSDYWDPFIR SPVDLEDDEL GNGTRWALLV |
| Length: 487 amino acids | AGSKGYQSYR HQANVCHAYQ ILKRGGLKDE |
| | NIVVFMYDDI ATCDENPRPG TIIHHPEGGD |
| | VYAGVPKDYT GDAVTADNFF AVILGDKSSV |
| | KGGSGKVIDS KPDDRIFLYY TDHGAAGLLG |
| | MPEKPYVVAN DFVEVLKKKH AMGTYKEMVI |
| | YLEACESGSI FEGLLPEDLN IYAITSTKPE |
| | EPSYIIYCPD MNPPPPPEYT TCLGDTFSVA |
| | WMEDSETHNL KKESLAQQIN KVKERTSMFG |
| | TYANGSHVME YGTKVIKPEK VYLYQGYNPE |
| | TYANGSHVME YGTKVIKPEK VYLYQGYNPE |
| | TANLPANRIH FDKKMESVNQ RDGDLIYLWQ |
| | KYKRSSVSNR AEALKQMTET LRYMAHLDSS |
| | VDMIGVLLFG PQNGGSILRS SRGRGLPLVD |
| | DWDCLKSMTR LFEKHCGLLT EYGMKHMRAF |
| | ANICNNLVEE TEVEEAIIAT CSGKNIGPYA |
| | SLGAYSV |
| OaAEP1b | MGMAHHHHHH MQIFVKTLTG KTITLEVEPS |
| (oldenlandia affinis wild-type) | DTIENVKAKI QDKEGIPPDQ QRLIFAGKQL |
| SEQ ID NO: 11 | EDGRTLSDYN IQKESTLHLV LRLRGGARDG |
| Length: 537 amino acids | DYLHLPSEVS RFFRPQETND DHGEDSVGTR |
| | WAVLIAGSKG YANYRHQAGV CHAYQILKRG |
| | GLKDENIVVF MYDDIAYNES NPRPGVIINS |
| | PHGSDVYAGV PKDYTGEEVN AKNFLAAILG |
| | NKSAITGGSG KVVDSGPNDH IFIYYTDHGA |
| | AGVIGMPSKP YLYADELNDA LKKKHASGTY |
| | KSLVFYLEAC ESGSMFEGIL PEDLNIYALT |
| | STNTTESSWC YYCPAQENPP PPEYNVCLGD |
| | LFSVAWLEDS DVQNSWYETL NQQYHHVDKR |
| | ISHASHATQY GNLKLGEEGL FVYMGSNPAN |
| | DNYTSLDGNA LTPSSIVVNQ RDADLLHLWE |
| | KFRKAPEGSA RKEEAQTQIF KAMSHRVHID |
| | SSIKLIGKLL FGIEKCTEIL NAVRPAGQPL |
| | VDDWACLRSL VGTFETHCGS LSEYGMRHTR |
| | TIANICNAGI SEEQMAEAAS QACASIP |

(Bolded area corresponds to the catalytically active core domain which is prepared
from the zymogen after activation at acetic pH; underlined bolded sequences may be
further processed during the activation process. Expression tags or mutations are
underlined.)

TABLE 3
Glutaminyl cyclases and their amino acid sequences

| | |
|---|---|
| Human glutaminyl cyclase (QC) | VSPSASAWPE EKNYHQPAIL NSSALRQIAE |
| SEQ ID NO: 12 | GTSISEMWQN DLQPLLIERY PGSPGSYAAR |
| Length: 339 amino acids | QHIMQRIQRL QADWVLEIDT FLSQTPYGYR |
| | SFSNIISTLN PTAKRHLVLA CHYDSKYFSH |
| | WNNRVFVGAT DSAVPCAMML ELARALDKKL |
| | LSLKTVSDSK PDLSLQLIFF DGEEAFLHWS |
| | PODSLYGSRH LAAKMASTPH PPGARGTSQL |
| | HGMDLLVLLD LIGAPNPTFP NFFPNSARWE |
| | ERLQAIEHEL HELGLLKDHS LEGRYFQNYS |
| | YGGVIQDDHI PFLRRGVPVL HLIPSPFPEV |
| | WHTMDDNEEN LDESTIDNLN KILQVFVLEY |
| | LHLHHHHHH |
| | (the underlined C-ter sequence is |
| | a His₆ tag added to facilitate |
| | purification) |
| Mouse glutaminyl cyclase (QC) | AWTQEKNHHQ PAHLNSSSLQ QVAEGTSISE |
| SEQ ID NO: 13 | MWQNDLRPLL IERYPGSPGS YSARQHIMQR |
| Length: 327 amino acids | IQRLQAEWVV EVDTFLSRTP YGYRSFSNII |
| | STLNPEAKRH LVLACHYDSK YFPRWDSRVF |
| | VGATDSAVPC AMMLELARAL DKKLHSLKDV |
| | SGSKPDLSLR LIFFDGEEAF HHWSPQDSLY |
| | GSRHLAQKMA SSPHPPGSRG TNQLDGMDLL |
| | VLLDLIGAAN PTFPNFFPKT TRWFNRLQAI |
| | EKELYELGLL KDHSLERKYF QNFGYGNIIQ |
| | DDHIPFLRKG VPVLHLIASP FPEVWHTMDD |
| | NEENLHASTI DNLNKIIQVF VLEYLHL |
| Glutaminyl cyclase | MAIGSVVFAA AGLLLLLLPP SHQQATAGNI |
| (Drosophila melanogaster) | GSQWRDDEVH FNRTLDSILV PRVVGSRGHQ |
| SEQ ID NO: 14 | QVREYLVQSL NGLGFQTEVD EFKQRVPVFG |
| Length: 340 amino acids | ELTFANVVGT INPQAQNFLA LACHYDSKYF |
| | PNDPGFVGAT DSAVPCAILL NTAKTLGAYL |
| | QKEFRNRSDV GLMLIFFDGE EAFKEWTDAD |
| | SVYGSKHLAA KLASKRSGSQ AQLAPRNIDR |
| | IEVLVLLDLI GARNPKFSSF YENTDGLHSS |
| | LVQIEKSLRT AGQLEGNNNM FLSRVSGGLV |
| | DDDHRPFLDE NVPVLHLVAT PFPDVWHTPR |
| | DNAANLHWPS IRNFNRVFRN FVYQYLKRHT |
| | SPVNLRFYRT |
| Glutaminyl cyclase | MATRSPYKRQ TKRSMIQSLP ASSSASSRRR |
| (Arabidopsis thaliana) | FISRKRFAMM IPLALLSGAV FLFFMPFNSW |
| SEQ ID NO: 15 | GQSSGSSLDL SHRINEIEVV AEFPHDPDAF |
| Length: 300 amino acids | TQGLLYAGND TLFESTGLYG KSSVRKVDLR |
| | TGKVEILEKM DNTYFGEGLT LLGERLFQVA |
| | WLTNTGFTYD LRNLSKVKPF KHHMKDGWGL |
| | ATDGKALFGS DGTSTLYRMD PQTMKVTDKH |
| | IVRYNGRESD CIARISPKDG SLLGWILLSK |
| | LSRGLLKSGH RGIDVLNGIA WDSDKQRLFV |
| | TGKLWPKLYQ ILKLQASAKS GNYIEQQCLV |
| Glutaminly cyclase (Conus frigidus) | MMEKVTTAAT YVRLLLLCSA VASNRALQNL |
| SEQ ID NO: 16 | GCGSLTSQYT VDNLSNLTVG MSDDGLRKKA |
| Length: 345 amino acids | LPPLLKPRVS GRRGNFNVRN SIIKWMRREG |
| | WSVQEDPFIA KTPYGWVRFS NVIATLNPRA |
| | ARRVVLACHY DSKLILFHGL SFVGATDSAV |
| | PCALLMDSAK KLRQVFQEKV ADASFQELTL |
| | QFIFFDGEEA YVQWSRSDSL YGARHLAQKW |
| | ASTPDPTAAG LNYLQTIGVF ILLDLIGSAD |
| | TRFANLFNQT AGVYAKLQSI EMCLTENGYL |
| | DATANPLPLF TSEQKQGTIE DDHLPFLRRG |
| | VPVVHLISTP FPSVWHKLSD NLHALDFQRT |
| | ENLARILRLF LVDLL |
| Glutaminly cyclase | MARERRDSKA ATFFCLAWTL CLALPGFPQH |
| (Sistrurus tergeminus) | VSGREDRADW TQEKYSHRPT ILNATCILQV |
| SEQ ID NO: 17 | TSQTNVNRMW QNDLHPILIE RYPGSPGSYA |
| Length: 368 amino acids | VRQHIKHRLQ GLQAGWLVEE DTFQSHTPYG |
| | YRTFSNIIST LNPLAKRHLV IACHYDSKYF |
| | PPQLDGKVFV GATDSAVPCA MMLELARSLD |
| | RQLSFLKQSS LPPKADLSLK LIFFDGEEAF |
| | VRWSPSDSLY GSRSLAQKMA STPHPPGARN |
| | TYQIQGIDLF VLLDLIGARN PVFPVYFLNT |
| | ARWFGRLEAI ERNLYDLGLL NNYSSERQYF |
| | RSNLRRHPVE DDHIPFLRRG VPILHLIPSP |
| | FPRVWHTMED NEENLDKPTI DNLSKILQVF |
| | VLEYLNLG |
| Bacterial glutaminyl cyclase | MPRLVPALLL ILALLPAMAV ARDPVPTQGY |
| (Xanthomonas campestris) | RVVKRYPHDT TAFTEGLFYL RGHLYESTGE |
| SEQ ID NO: 18 | TGRSSVRKVD LETGRILQRA EVPPPYFGEG |
| Length: 267 amino acids | IVAWRDRLIQ LTWRNHEGFV YDLATLTPRA |
| | RFRYPGEGWA LTSDDSHLYM SDGTAVIRKL |
| | DPDTLQQVGS IKVTAGGRPL DNLNELEWVN |
| | GELLANVWLT SRIARIDPAS GKVVAWIDLQ |
| | ALVPDADALT DSTNDVLNGI AFDAEHDRLF |
| | VTGKRWPMLY EIRLTPLPHA AAGKHAQ |

TABLE 4
Protein Sequences and their related mass spectrometry data

| | |
|---|---|
| Ubi-NQL-His | MQIFVKTLTG KTITLEVEPS DTIENVKAKI QDKEGIPPDQ |
| SEQ ID NO: 19 | QRLIFAGKQL EDGRTLSDYN IQKESTLHLV LRLRGGNQLH |
| (calc. 9743, obvs. 9746) | HHHHH |
| Amino acid sequence | |
| Length: 85 amino acids | |
| GI-Ubi | GIMQIFVKTL TGKTITLEVE PSDTIENVKA KIQDKEGIPP |
| SEQ ID NO: 20 | DQQRLIFAGK QLEDGRTLSD YNIQKESTLH LVLRLRGG |
| (calc. 8735, obvs. 8735) | |
| Amino acid sequence | |
| Length: 78 amino acids | |
| GI-GFP | MGIGSKKVSK GEELFTGVVP ILVELDGDVN GHKFSVRGEG |
| SEQ ID NO: 21 | EGDATNGKLT LKGICTTGKL PVPWPTLVTT LTYGVQCFSR |
| (calc. 28344, obvs. 28341) | YPDHMKRHDF FKSAMPEGYV QERTISFKDD GTYKTRAEVK |
| Amino acid sequence | FEGDTLVNRI ELKGIDFKED GNILGHKLEY NFNSHNVYIT |
| Length: 254 amino acids | ADKQKNGIKA NFKIRHNVED GSVQLADHYQ QNTPGIDPGV |
| | LLPDNHYLST QSVLSKDPNE KRDHMVLLEF VTAAGITHGM |
| | DELYKGSGHH HHHH |
| GI-GGGSGGGS-GFP | MGIGSGGGSG GGSKKVSKGE ELFTGVVPIL VELDGDVNGH |
| SEQ ID NO: 22 | KFSVRGEGEG DATNGKLTLK FICTTGKLPV PWPTLVTTLT |
| (calc. 28803, obvs. 28803) | YGVQCFSRYP DHMKRHDFFK SAMPEGYVQE RTISFKDDGT |
| Amino acid sequence | YKTRAEVKFE GDTLVNRIEL KGIDFKEDGN ILGHKLEYNF |
| Length: 261 amino acids | NSHNVYITAD KQKNGIKANF KIRHNVEDGS VQLADHYQQN |
| | TPIGDGPVLL PDNHYLSTQS VLSKDPNEKR DHMVLLEFVT |
| | AAGITHGMDE LYKGSHHHHH H |
| DARPin-NQL | MHHHHHHGSD LGKKLLEAAR AGQDDEVRIL MANGADVNAK |
| SEQ ID NO: 23 | DFYGITPLHL AAAYGHLEIV EVLLKHGADV NAHDWNGWTP |
| (calc. 18846, obvs. 18849) | LHLAAKYGHL EIVEVLLKHG ADVNAIDNAG KTPLHLAAAH |
| Amino acid sequence | GHLEIVEVLL KYGADVNAQD KFGKTPFDLA IDNGNEDIAE |
| Length: 175 amino acids | VLQKAAKLGS GSNQL |
| GI-DARPin | MGISSHHHHH HGSDLGKKLL EAARAGQDDE VRILMANGAD |
| SEQ ID NO: 24 | VNAKDFYGIT PLHLAAAYGH LEIVEVLLKH GADVNAHDWN |
| (calc. 18530, obvs. 18533) | GWTPLHLAAK YGHLEIVEVL LKHGADVNAI DNAGKTPLHL |
| Amino acid sequence | AAAHGHLEIV EVLLKYGADV NAWDKFGKTP FDLAIDNGNE |
| Length: 173 amino acids | DIAEVLQKAA KLN |
| Z_{EGFR}-NQL | MKKGSSHHHH HHLQVDNKFN KEMWAAWEEI RNLPNLNGWQ |
| SEQ ID NO: 25 | MTAFIASLVD DPSQSANLLA EAKKLNDAQA PKVDGSGSNQ |
| (calc. 9085, obvs. 9086) | L |
| Amino acid sequence | |
| Length: 81 amino acids | |
| Z_{EGFR}-Fc-NQL | MKKGSSHHHH HHLQVDNKFN KEMWAAWEEI RNLPNLNGWQ |
| SEQ ID NO: 26 | MTAFIASLVD DPSQSANLLA EAKKLNDAQA PKVDGSGSDK |
| (for monomer, calc. 34759, obvs. 34765) | THTCPPCPAP ELLGGPSVFL FPPKPKDTLM ISRTPEVTCV |
| Amino acid sequence | VVDVSHEDPE VKFNWYVDGV EVHNAKTKPR EEQYNSTYRV |
| Length: 310 amino acids | VSVLTVLHQD WLNGKEYKCK VSNKALPAPI EKTISKAKGQ |
| | PREPQVYTLP PSRDELTKNQ VSLTCLVKGF YPSDIAVEWE |
| | SNGQPENNYK TTPPVLDSDG SFFLYSKLTV DKSRWQQGNV |
| | FSCSVMHEAL HNHYTQKSLS LSPGKGSNQL |

### Example 1: Demonstration of the PAL-QC Cascade Scheme in Model Peptide Ligation Reactions

By catalyzing N-terminal pGlu formation, QC is involved in the maturation of many bioactive peptides and proteins (Busby, Jr., et al., J. Biol. Chem. 262, 8532-8536 (1987); Schilling, S., et al., Biochemistry 41, 10849-10857 (2002); Seifert, F., et al., Biochemistry 48, 11831-11833 (2009)). The efficiency of QCs from different organisms at catalyzing the unimolecular lactamization reaction is ˜10⁵ M⁻¹·S⁻¹ (Seifert, F., et al., Biochemistry 48, 11831-11833 (2009)), while that of PALs in catalyzing the bimolecular ligation reactions is ˜10⁴ M⁻¹·S⁻¹. Wang, Z., et al.; Theranostics 11, 5863-5875 (2021)). This makes QC a particularly attractive enzyme to trap the released glutaminyl leaving group. We first showed that QC efficiently converted the N-terminal Gln to pGlu in four synthetic peptides of the sequence QXGSA (X=L, I, F or V, which are favored by PALs as the P2′ amino acid) (FIG.7). Consistent with previous studies, the presence of a large hydrophobic residue like X at the second position of a glutaminyl peptide did not negatively affect QC on its ability to catalyze the lactamization reaction. Indeed, at 0.0001 eq to the substrate, QC was able to complete the reaction in less than 30 min (pH 7). The reactions of QI-, QL-and QF-peptides had similar rates while that of QV-peptide was about 30% slower (FIG. 7). Since Leu is the most favored P2′ residue of PALs, we chose Asn-Gln-Leu as the tripeptide recognition motif in all acyl donor substrates used in this study.

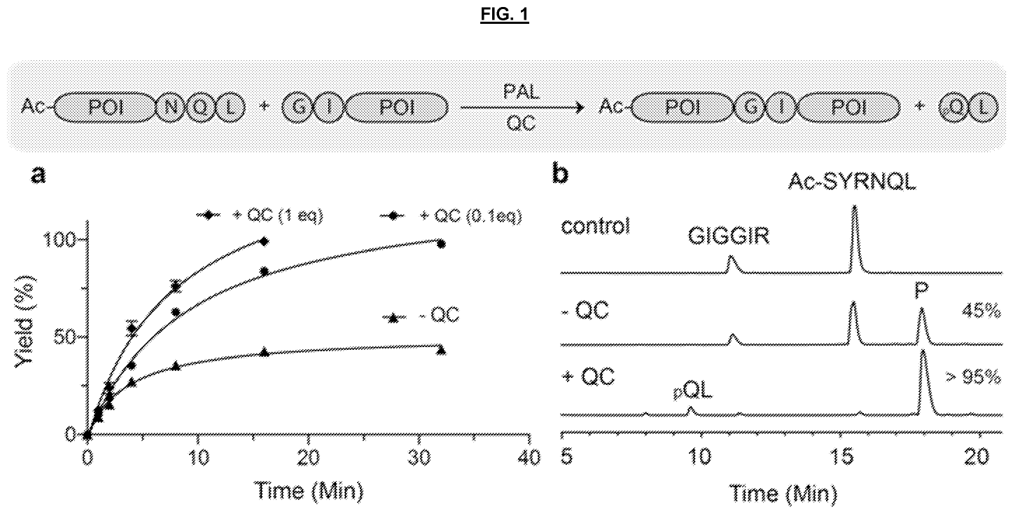

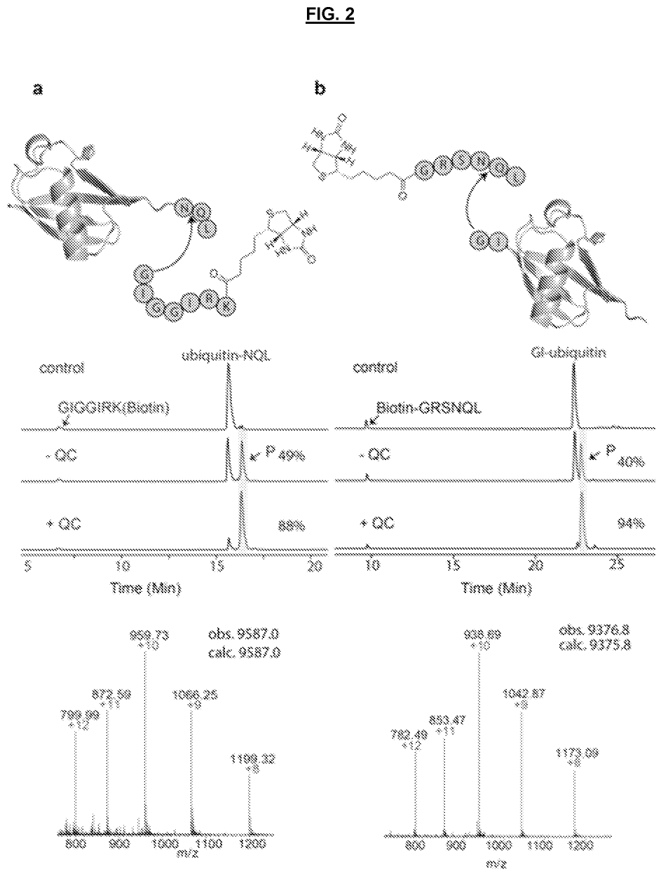

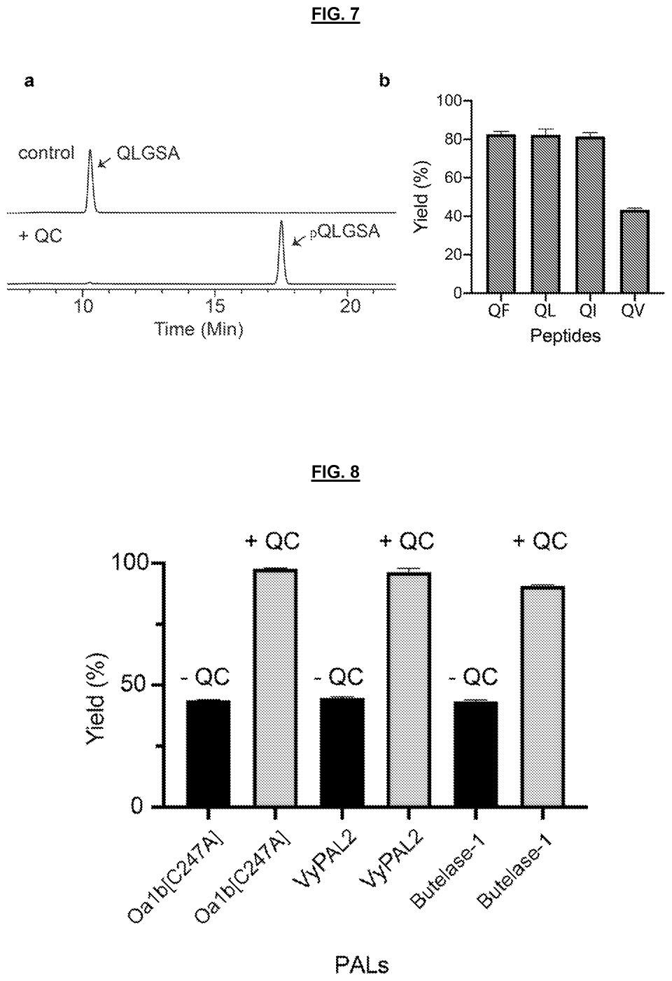

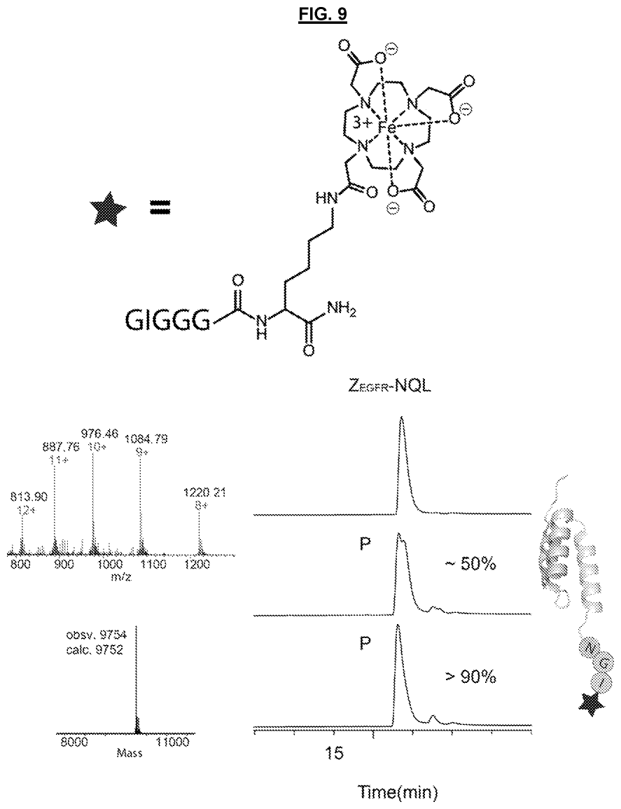

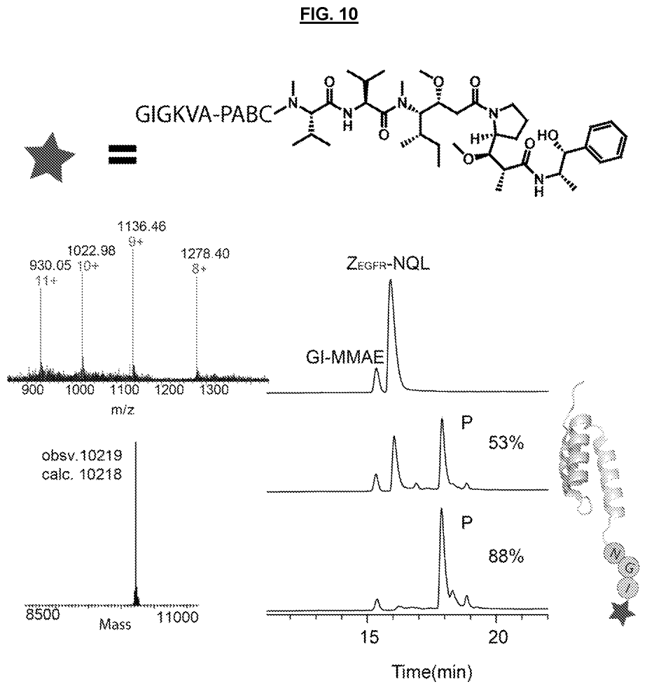

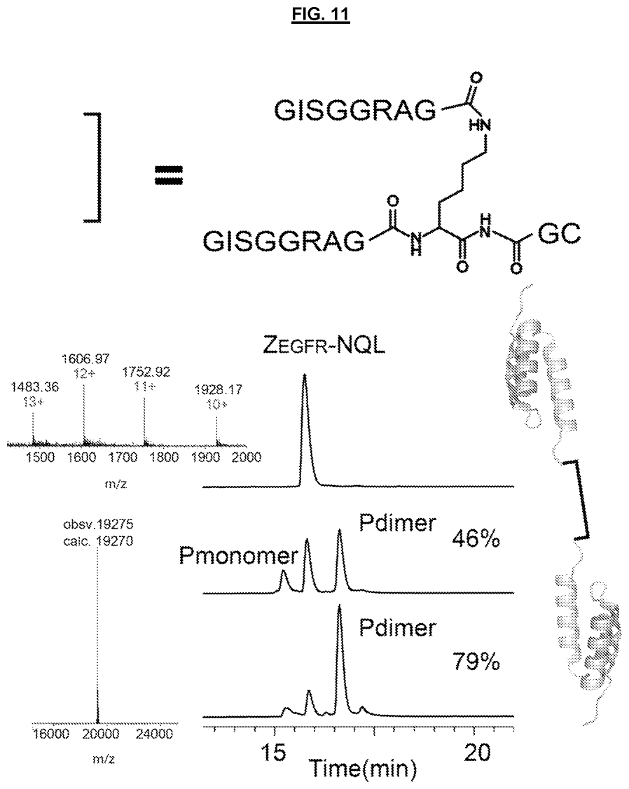

Then we set out to test the cascade enzymatic scheme of the invention in a model ligation reaction using Ac-SYRNQL (5 mM) as the acyl donor and GIGGIR (1 eq) as the acyl acceptor. At a PAL-to-substrate molar ratio of 0.0005:1, the reaction by OaAEP1b-C247A at pH 7 gave the product in ˜45% yield when the reaction reached equilibrium at ˜30 min (FIG. 1a). In contrast, the addition of 0.00005 eq of QC increased the ligation yield to >95% in 32 min (FIGS. 1a and 1b). This drastic yield increase indicated that a very low amount of QC (0.005 mol % to the substrate and 1/10 eq to PAL) was sufficient to quench the released QL dipeptide through pGlu formation. This provided a clear validation of the PAL-QC coupled reaction scheme. When performing the ligation reaction using 0.05 mol % QC (i.e., QC: PAL=1:1), the reaction reached equilibrium faster, but the final yield was almost the same (FIG. 1a). Because the reaction was fast enough at 1/10 QC to PAL, this ratio was used in all following studies. The ligation reaction was also conducted using VyPAL2 or butelase-1 under the same conditions. A similar drastic increase of the product yield was observed when the reaction was done in the presence of QC, demonstrating that QC is compatible with all the three most useful PALs (FIG. 8).

### Example 2: Use of the Cascade Enzymatic Scheme for Protein N- and C-Terminal Labelling

Ubiquitin was then used as a model protein to demonstrate the method in protein labelling reactions. Two recombinant ubiquitin variants, Gl-ubiquitin and ubiquitin-NQL-His₆, were prepared for N- and C-terminal labelling with two biotinylated synthetic peptides, biotin-GRSNQL and GIGGIRK (biotin), respectively. 500 μM of the ubiquitin substrate protein and 1.2 eq of the biotin peptide were used in both ligation reactions which were conducted at pH 7 and 37° C. with 0.5 μM OaAEP1b-C247A (0.001 eq).

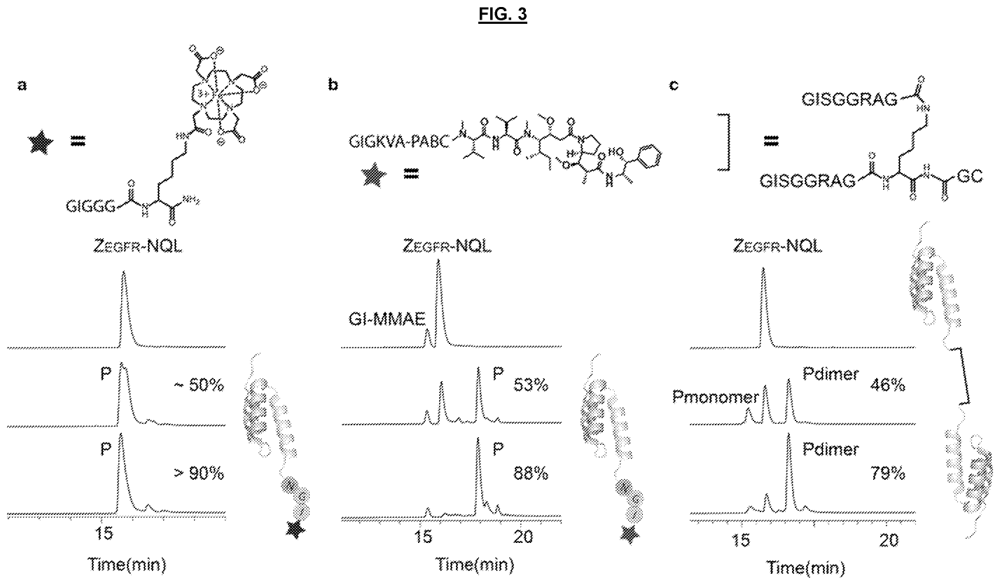

For the ligation reaction of ubiquitin-NQL-His6 with GIGGIRK (biotin), the yield increased from 40 to 94% when 0.0001 eq of QC was added (FIG. 2). Similarly, the yield of ligation between biotin-GRSNQL and Gl-ubiquitin increased from 49% to 88% with the addition of QC. The huge enhancement of the protein C- and N-terminal labelling efficiency by QC further validated the method in protein-to-peptide ligation reactions.

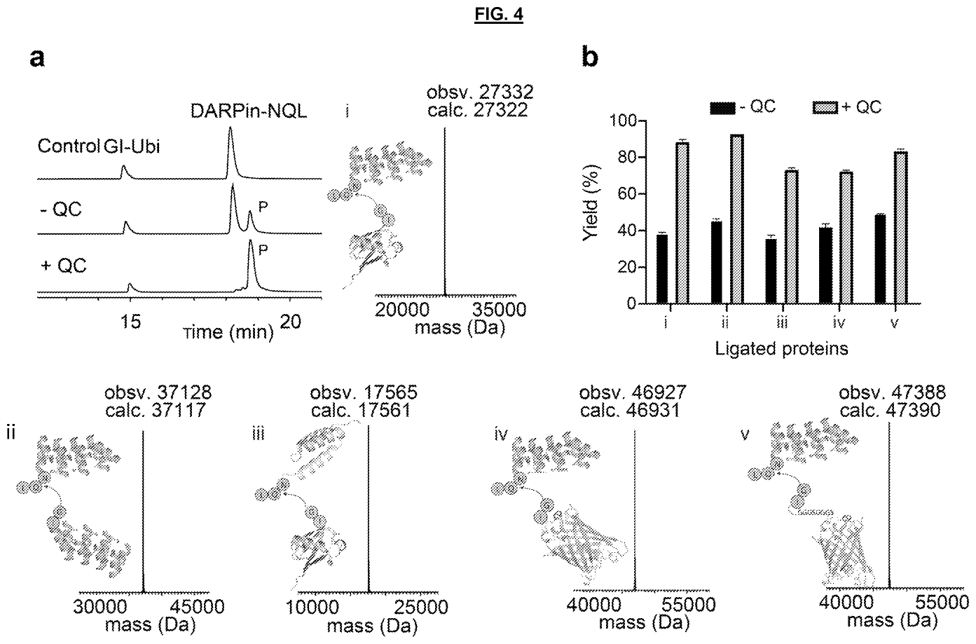

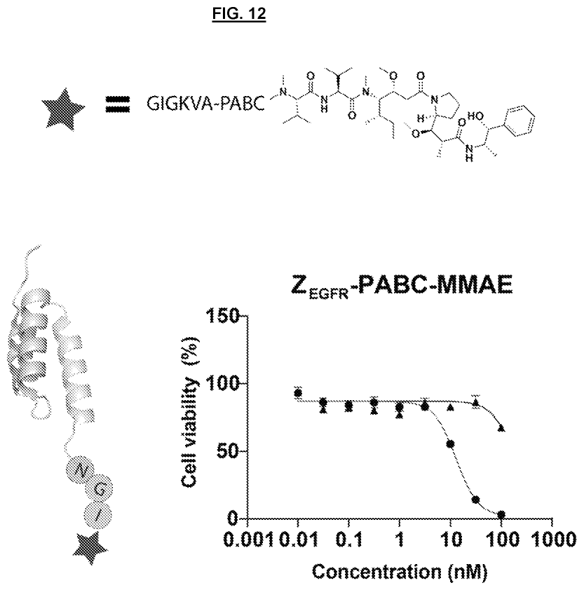

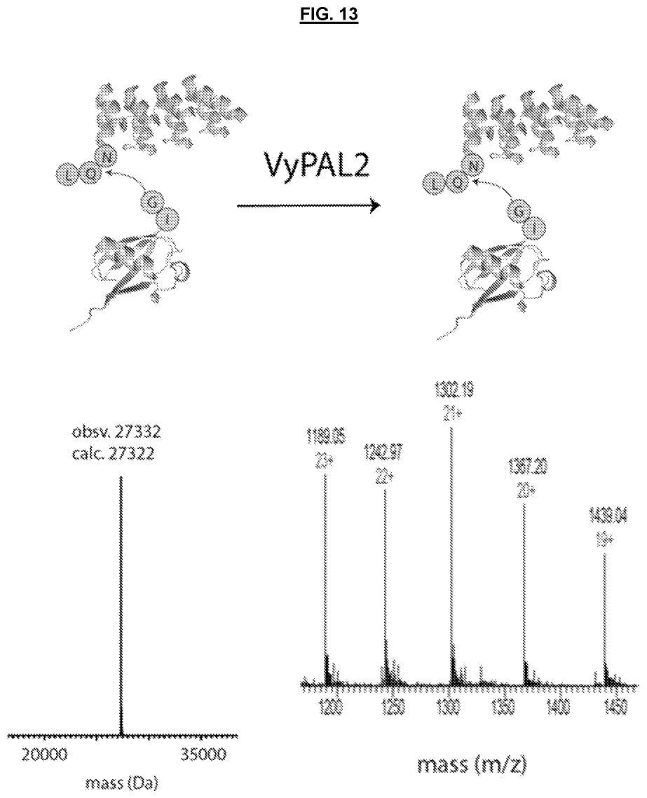

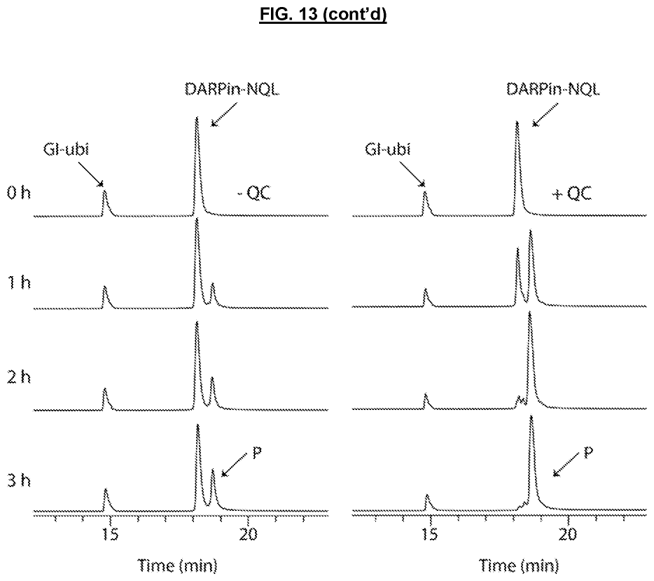

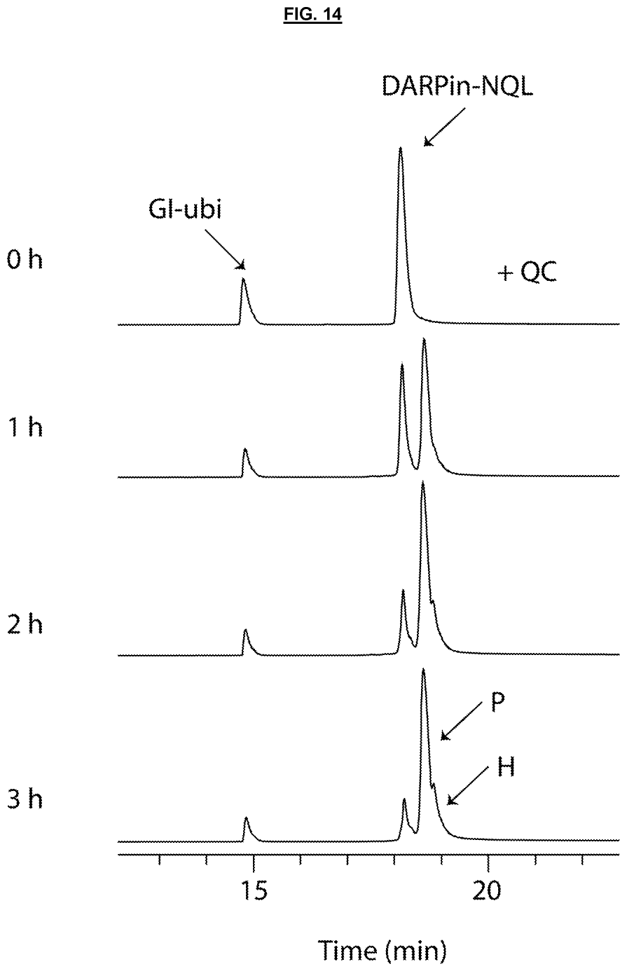

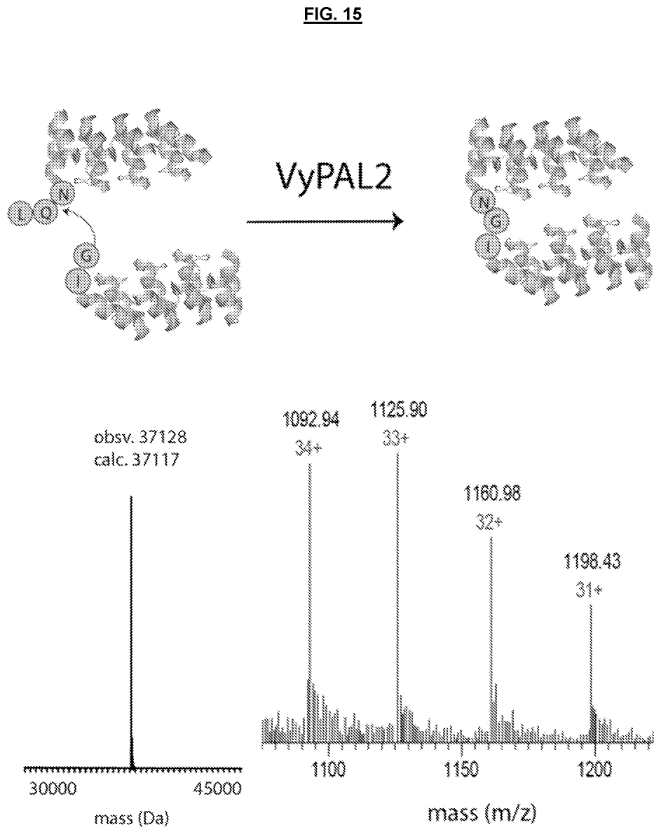

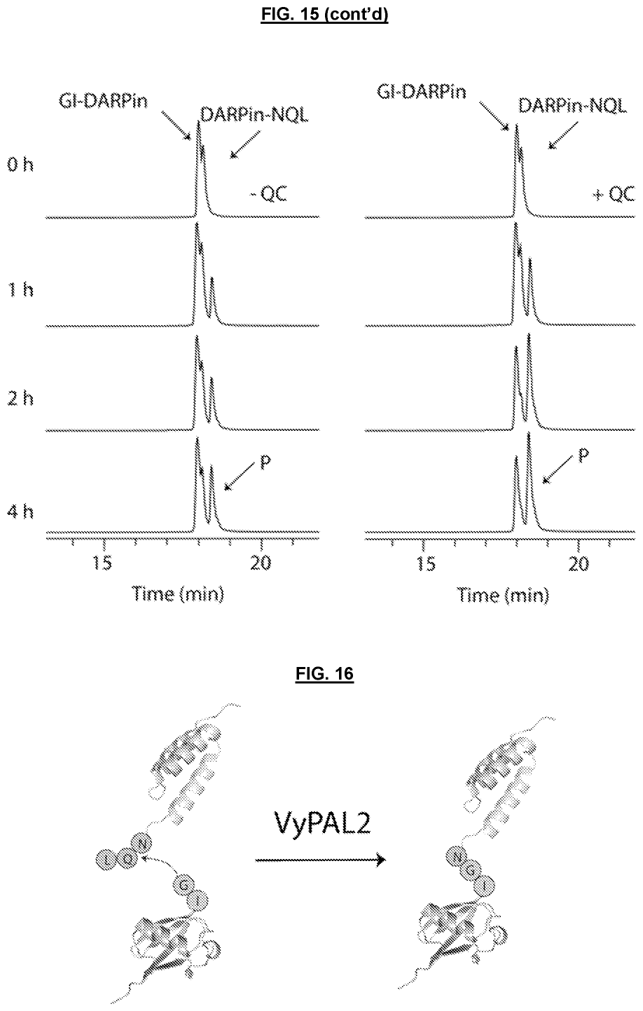

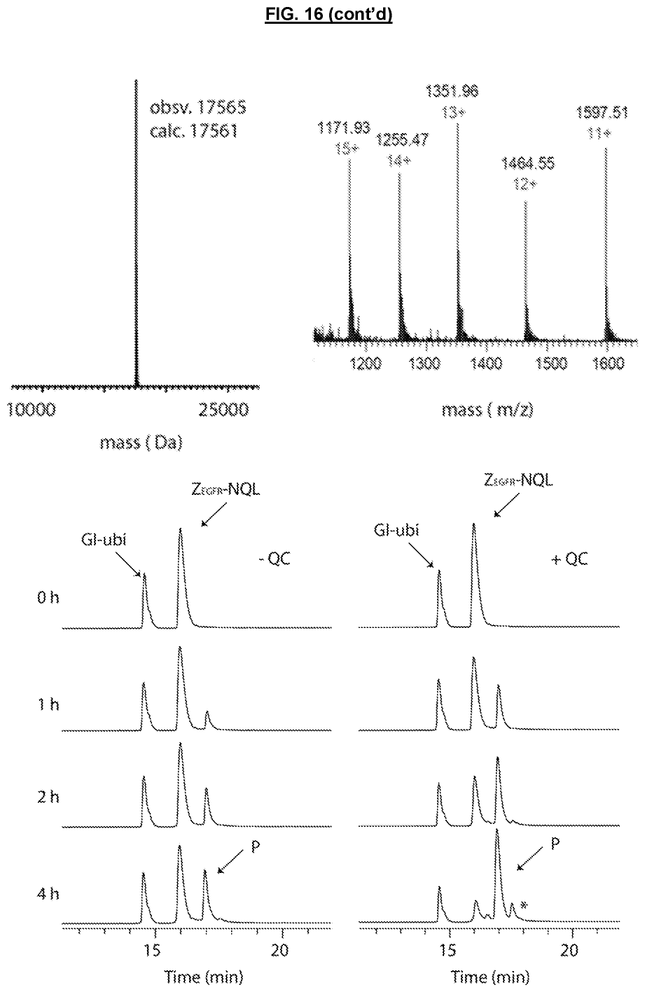

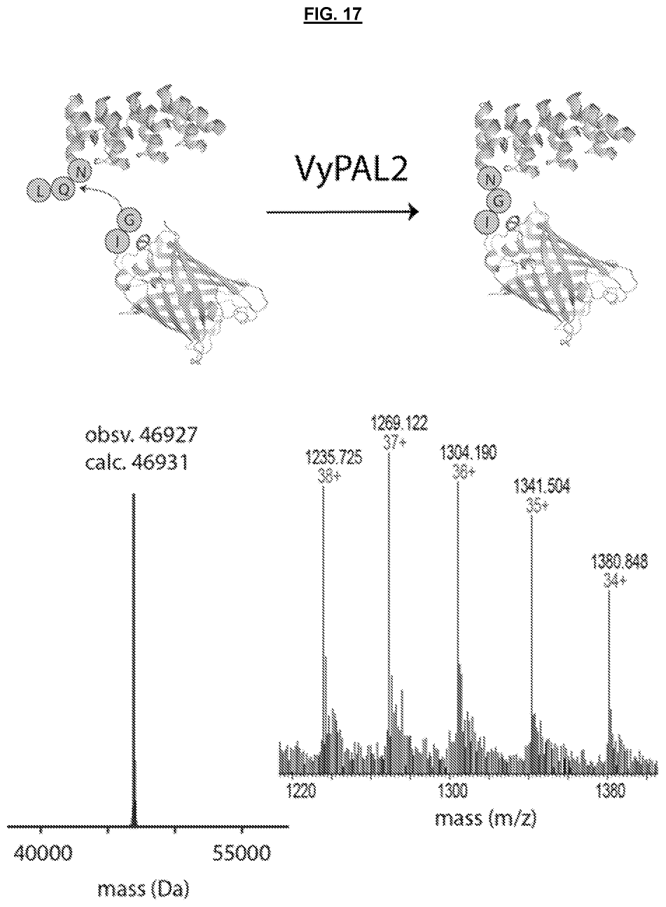

Next, an anti-EGFR affibody protein Z_{EGFR} (Ståhl, S., et al.; Trends Biotechnol. 35, 691-712 (2017)) was C-terminally labelled with functional moieties of potential diagnostic and therapeutic interest. Two special peptides, GIGGGK[Fe(DOTA)] and GIGKVA-PABC-MMAE, were prepared and used as the acyl acceptor substrates for ligation with Z_{EGFR}-NQL (FIGS. 3a and 3b). As the most powerful chelators, DOTA and its derivatives form very stable complexes with certain metal ions (Viola-Villegas, N., et al., Coordination Chemistry Reviews 2009, 253, 1906-1925 (2009)). Disease-targeting proteins can be conjugated with DOTA complexes containing a radionuclide for diagnostic or theranostic applications (Sgouros, G., et al., Nat. Rev. Drug Dis. 19, 589-608 (2020)). In this study, the DOTA complex contained the non-radioactive Fe³⁺ ion for demonstration purpose only. MMAE or monomethyl auristatin E, a highly potent cytotoxic agent, is a common drug payload in antibody-drug conjugates (Chen, H., et al.; Molecules 22, 1281 (2017)), which is often linked to the antibody through PABC—a self-immolative linker for payload release (Doronina, S., et al.; Bioconjugate Chem. 17, 114-124 (2006)). As seen in FIG. 3, the cascade enzymatic method afforded the Z_{EGFR}-[Fe(DOTA)] and Z_{EGFR}-MMAE conjugates in 90% and 88% yields, respectively, whereas in the absence of QC, the yield was only about 50%. The [Fe(DOTA)] complex was stable during HPLC purification and ESI-MS measurement as intact molecular ions were observed of the GIGGGK[Fe(DOTA)] peptide and the Z_{EGFR}-[Fe(DOTA)] conjugate. The Z_{EGFR}-MMAE conjugate was shown to have high cytotoxicity against A431 cells which over-express EGFR but relatively low cytotoxicity against MCF-7 cells which express very low levels of EGFR (FIG. 12). Clearly, these two examples show that our PAL-QC coupled cascade scheme can offer a safer manufacturing process for bioconjugates containing radioactive or toxic payloads as no large excess of reactants is needed for a high-yielding reaction, which minimizes risks of exposure to these hazardous substances.

A C-terminally linked dimer of the Z_{EGFR} protein was also prepared by ligating it with a bivalent peptide substrate containing two Gly-Ile dipeptide acyl acceptors (FIG. 3c). This is a more stringent test of our method, because both nucleophilic sites in the bivalent peptide need to be ligated with the protein. Gratifyingly, using only a slight excess of Z_{EGFR} (1.1 effective equivalence to the bivalent peptide), the cascade scheme gave the desired C-terminal dimer protein product in ca. 80%. In contrast, without QC, only ca. 46% of the C-terminal dimer was obtained and a significant amount of the mono-ligated intermediate was observed (FIG. 3c). The synthesis of such a parallel protein dimer illustrates the utility of our method in preparing such unusual protein conjugates.

### Example 3: Use of the Cascade Enzymatic Reaction Scheme for Protein-Protein Ligation

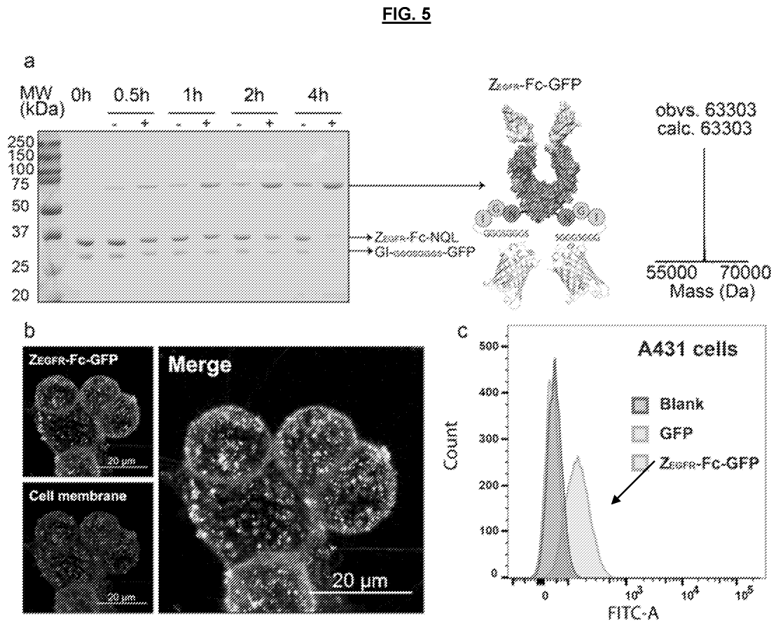

Several proteins were then selected—GFP, ubiquitin, DARPin9-26 (Steiner, D., et al.; J. Mol. Biol. 382, 1211-1227 (2008); Jost, C., et al.; Structure 21, 1979-1991 (2013)) and an anti-EGFR affibody Z_{EGFR} (Ståhl, S., et al.; Trends Biotechnol. 35, 691-712 (2017))—to determine whether the method of the invention could be further extended to protein-protein ligation reactions. We first conducted ligation of DARPin-NQL (400 μM) with GI-ubiquitin (1.8 eq) using VyPAL2 (0.001 eq) at pH 7 and 37° C. Without QC, the reaction yielded the product in 39% in 3 h, whereas the addition of QC increased the yield to 91% (FIG. 4a). Interestingly, we found that, for this DARPin-to-ubiquitin ligation reaction, VyPAL2 was a better ligase than OaAEP1b-C247A because the latter produced a small amount of hydrolysis product DARPin-N-OH (FIG. 14). This phenomenon was not observed in neither peptide-peptide ligation nor peptide-protein ligation reactions. It seems that OaAEP1b-C247A has residual hydrolase activity which could be exacerbated in the difficult, entropically demanding protein-protein ligation reaction. Therefore, VyPAL2 was used for all subsequent inter-protein ligation reactions.

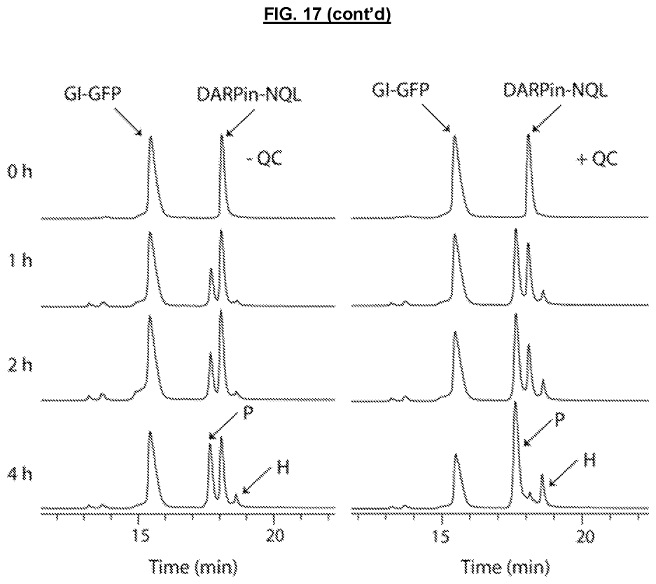

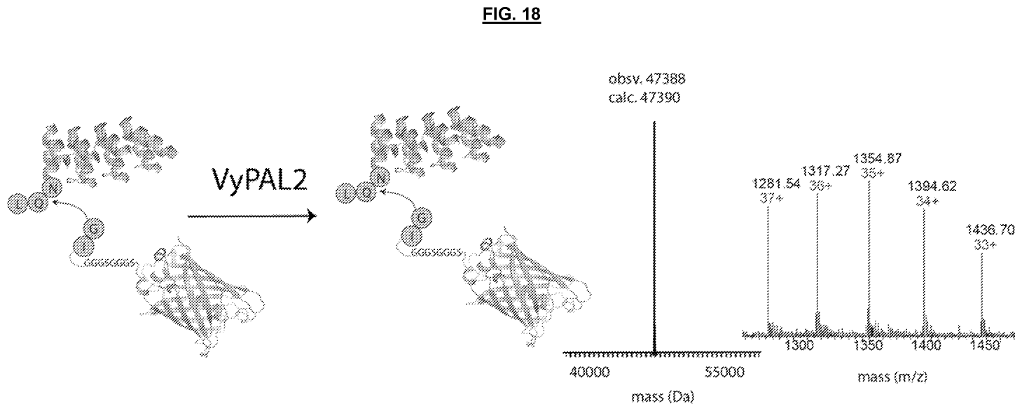

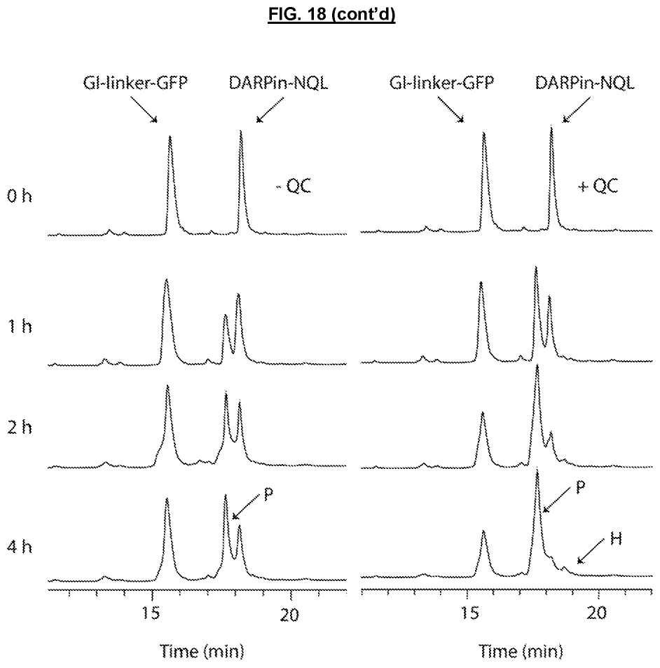

Similarly, ligation of DARPin-NQL (1 eq) with GI-DARPin (1.8 eq) afforded the tandem-linked DARPin-NGI-DARPin in 47% (without QC) and 95% (with QC) (FIG. 4b). Ligation of Z_{EGFR}-NQL with GI-ubiquitin afforded the product in 37% (without QC) and 76% (with QC). DARPin-NQL was also ligated with the much larger GFP protein, GI-GFP, to produce DARPin-NGI-GFP in 42% (without QC) and 74% (with QC). The proximity of the N-terminal GI nucleophile to the rigid B-barrel structure of the GFP protein might have hindered its accessibility, leading to the slightly lower ligation yield and some hydrolysis in this reaction (FIG. 17). Adding a longer spacer after the GI dipeptide improved the accessibility and consequently the ligation yields between DARPin and GI-GGGSGGGS-GFP-50% (without QC) and 85% (with QC). In all cases, the product yields were improved by 33-53% when using QC together with VyPAL2. This improvement is remarkably comparable to that observed in the model peptide ligations, which makes the method of the invention distinctly advantageous to all previous methods (Nguyen, G. K. T., et al.; Angew. Chem. Int. Ed. 54, 15694-15698 (2015); Cao, Y., et al.; Bioconj. Chem. 27, 2592-2596 (2016); Rehm, F. B.H., et al.; J. Am. Chem. Soc. 141 (43), 17388-17393 (2019); Tang, T. M. S., et al.; Chem. Sci. 11, 5881-5888 (2020); Rehm, F. B. H., et al.; Angew. Chem. Int. Ed. 2021, 60, 4004-4008 (2021)).

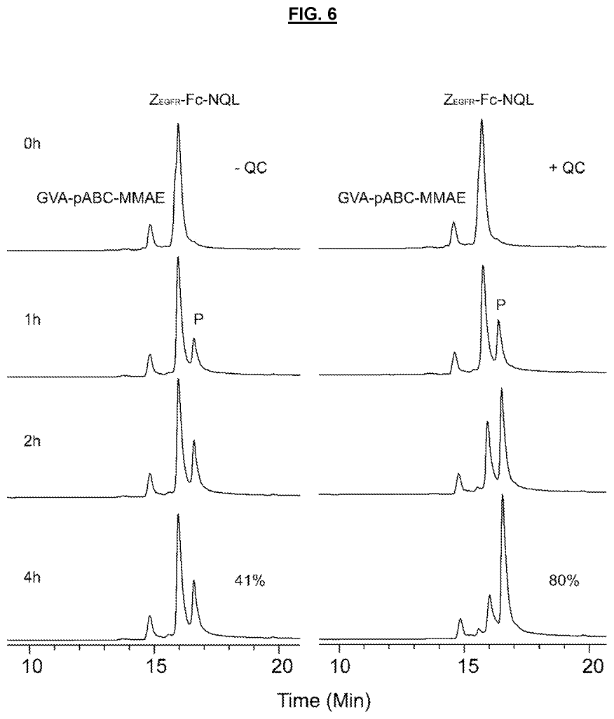

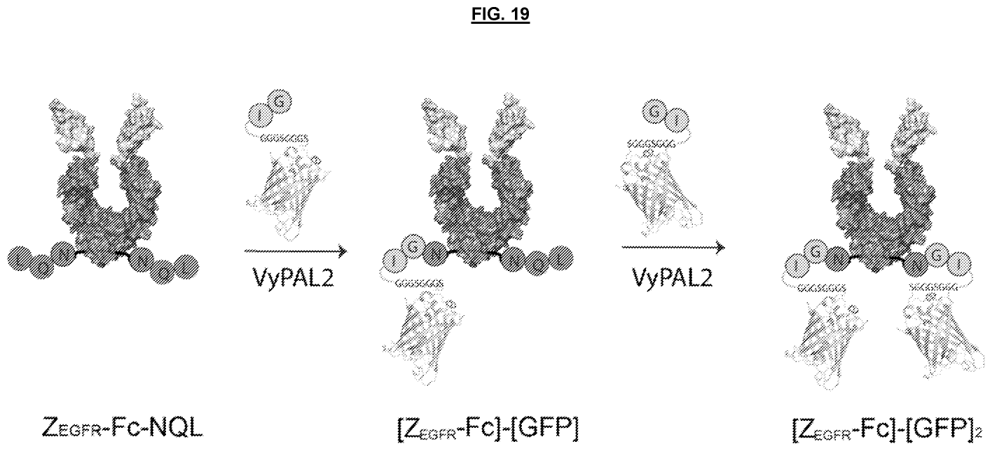

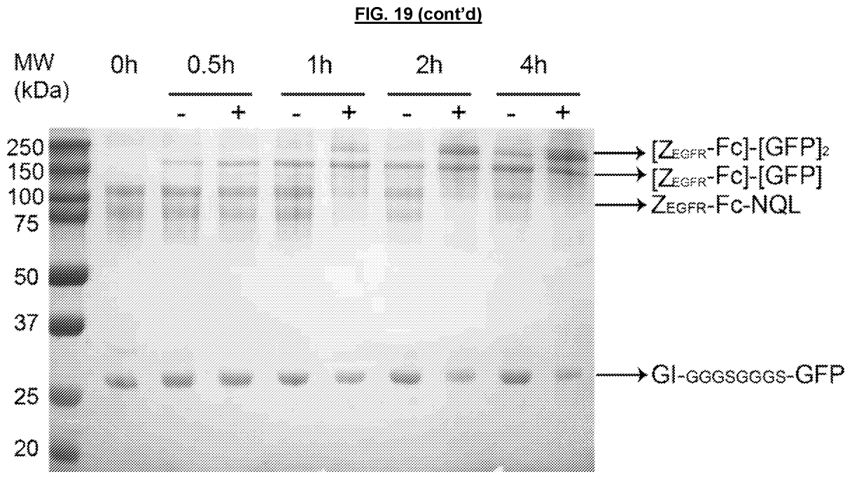

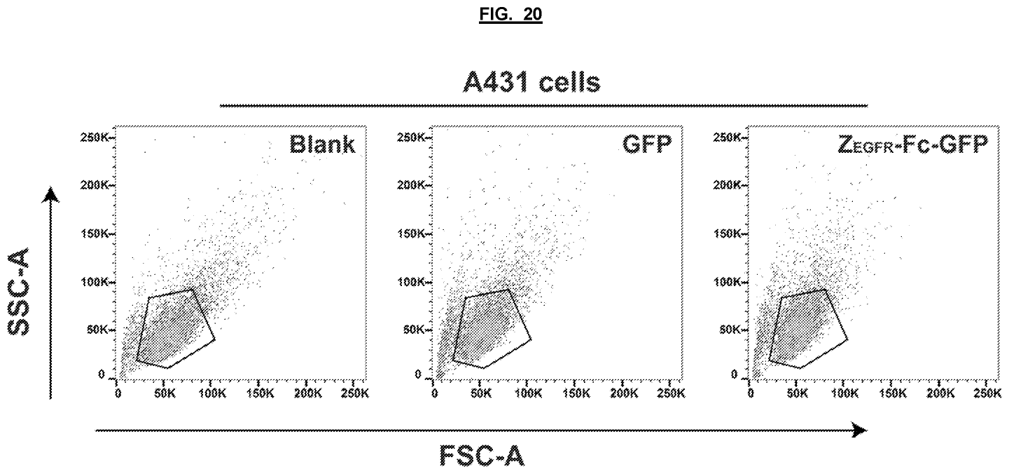

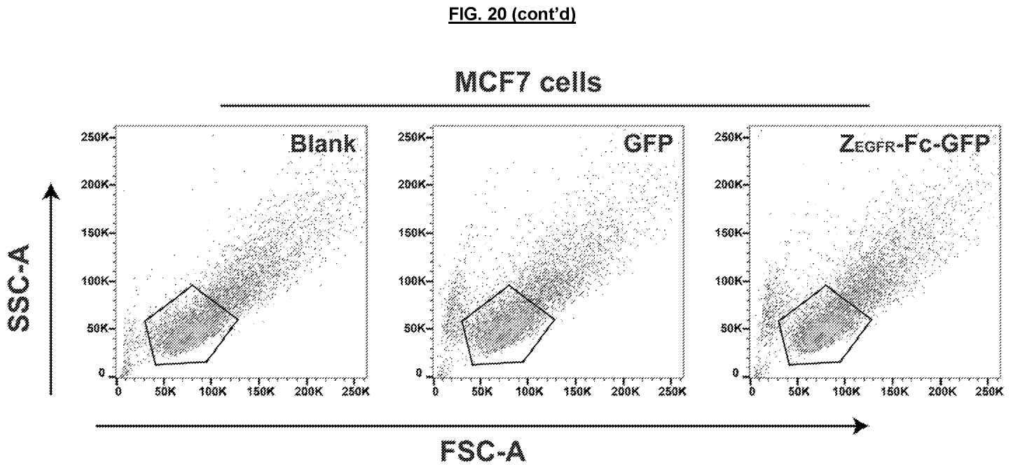

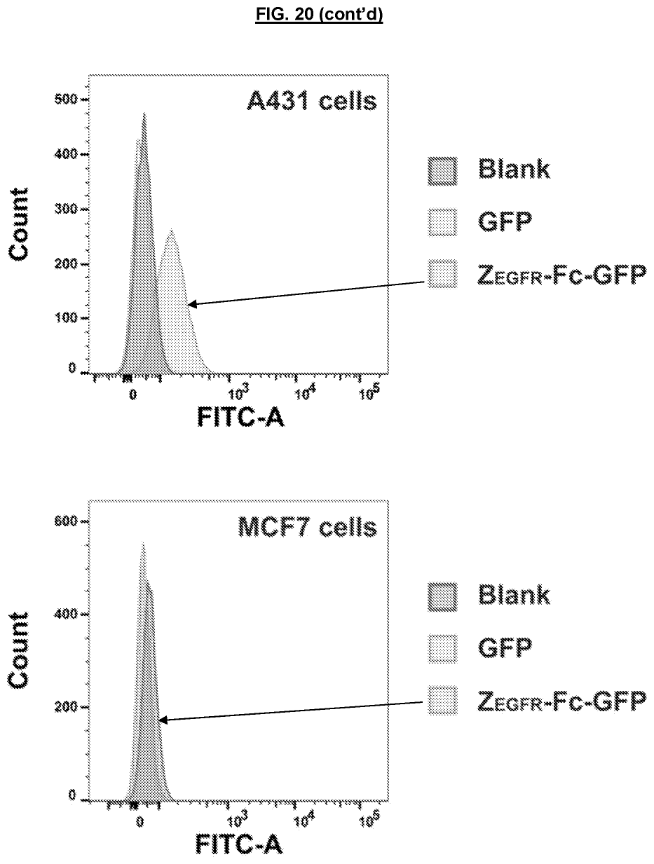

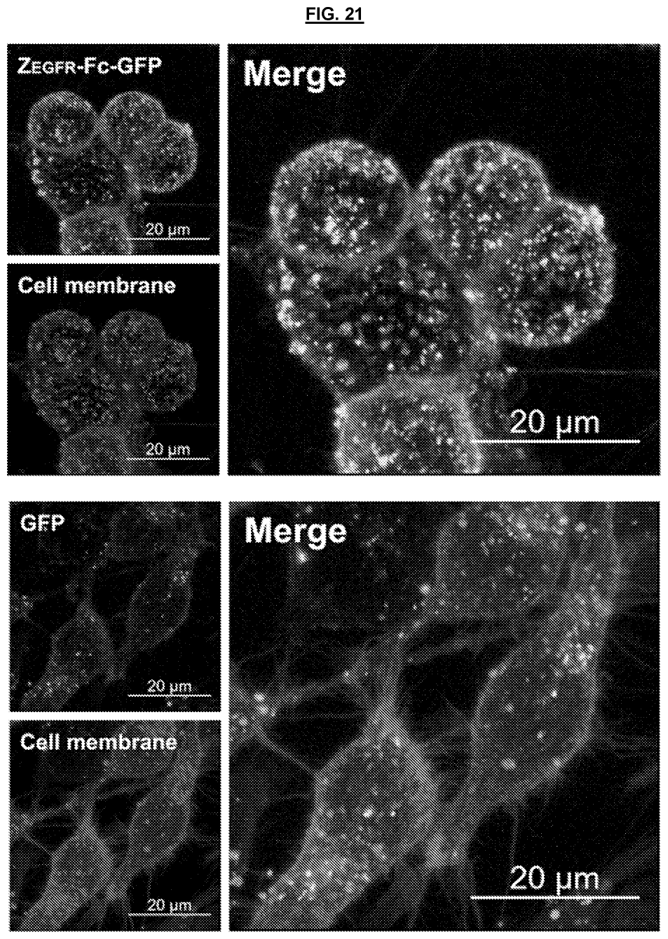

Next, Z_{EGFR}-FC-NQL, a large dimeric fusion protein (MW ˜68 kDa) composed of the affibody Z_{EGFR} and the Fc domain of IgG, was used to ligate with GI-GGGSGGGS-GFP (29 kDa) to get a very large protein product with a mass of ˜126 kDa. The ligation reaction between Z_{EGFR}-FC-NQL (200 μM) and the GFP protein (500 μM) reached ca. 90% yield in the presence of QC (FIG. 5a). This is remarkable considering the large sizes of the proteins and that the acceptor GFP protein substrate was used only at a 1.25 molar equivalence to the donor substrate Z_{EGFR}-Fc-NQL which, as a dimer, has two reaction sites. Furthermore, as seen from the non-reducing SDS-PAGE gel (FIG. 19), in the presence of QC, the final dual ligated product was predominant, whereas in the absence of QC, the mono-ligated product was predominant. FIG. 5b and c show the specific binding of dual-ligated product Z_{EGFR}-Fc-GFP towards A431 cells which overexpresses EGFR, indicating that the receptor binding activity of Z_{EGFR} and fluorogenicity of GFP were preserved after the ligation reaction. This example demonstrates that the VyPAL2-QC coupled method is also suitable for the preparation of large therapeutic protein conjugates.

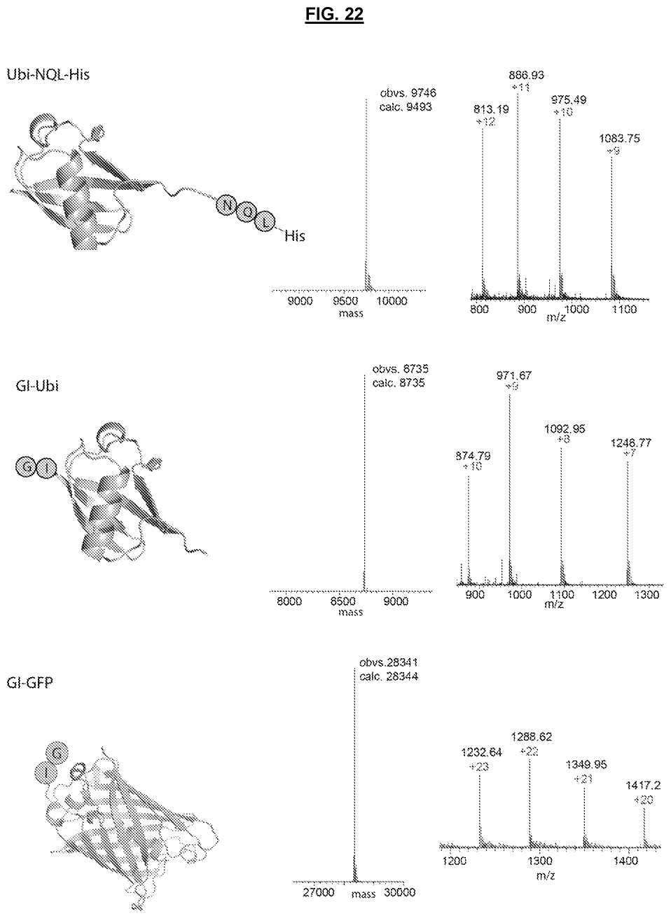

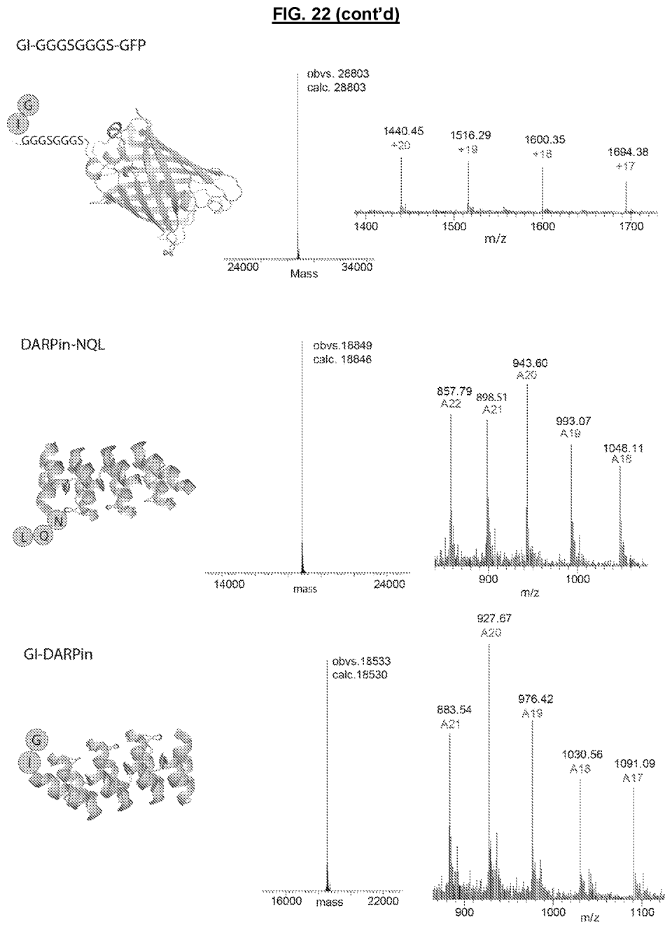

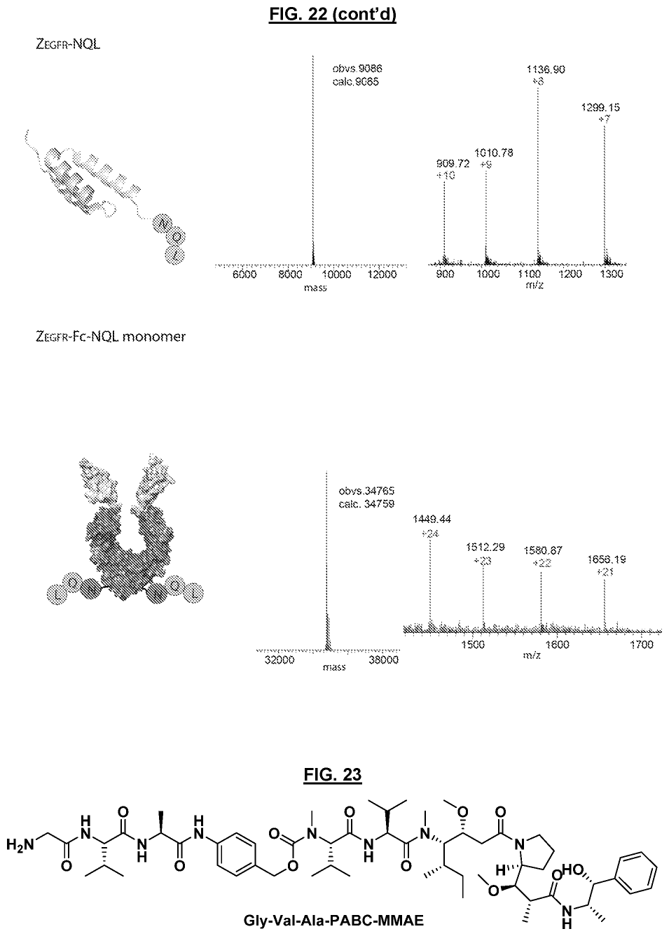

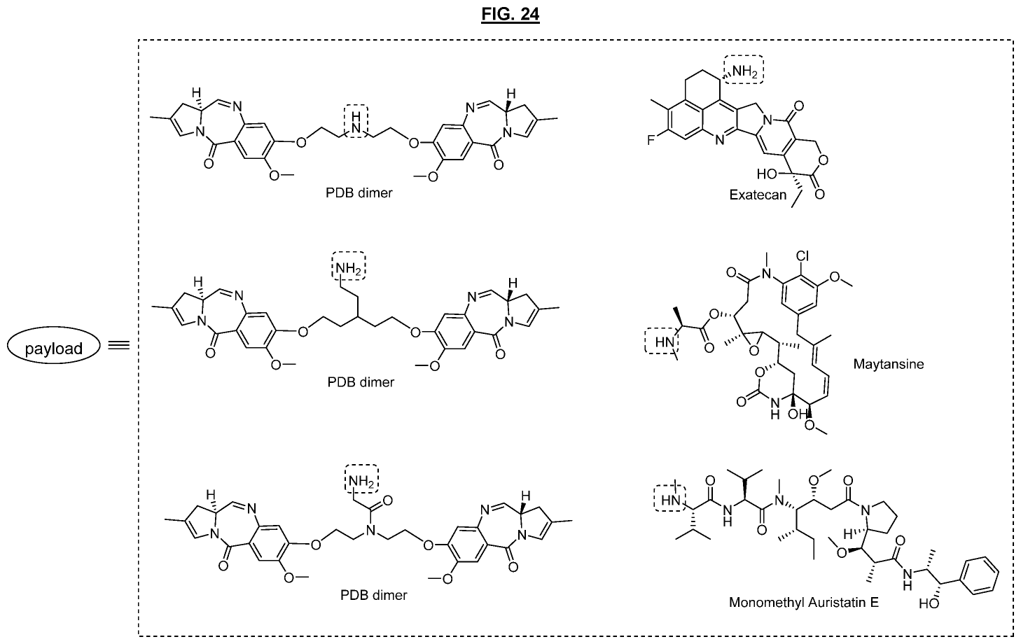

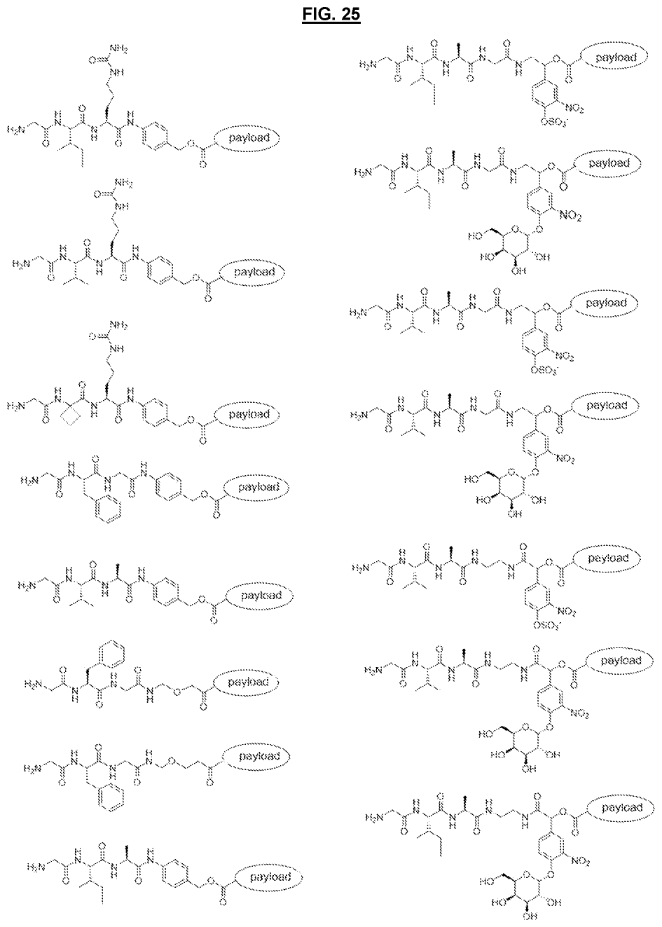

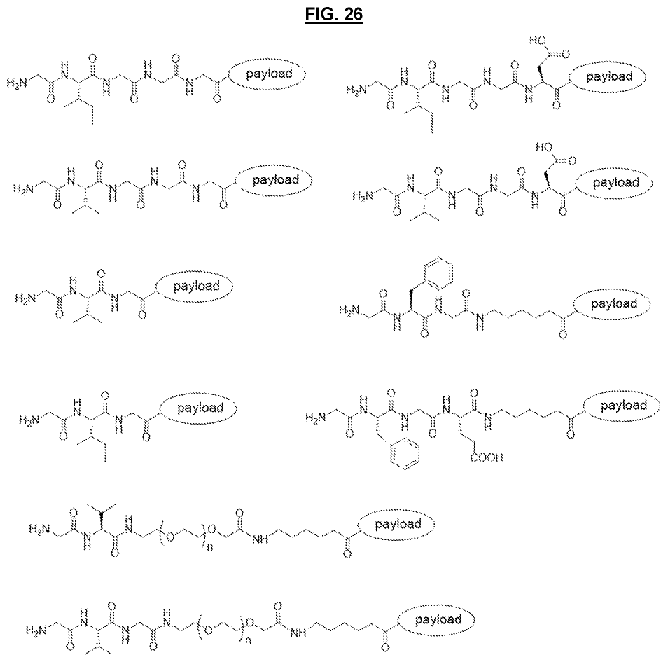

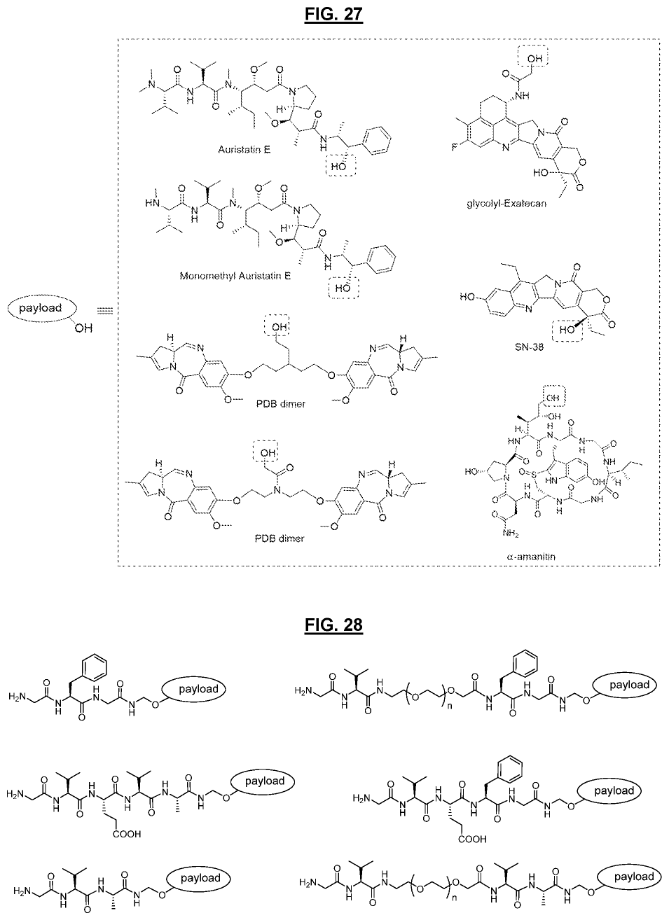

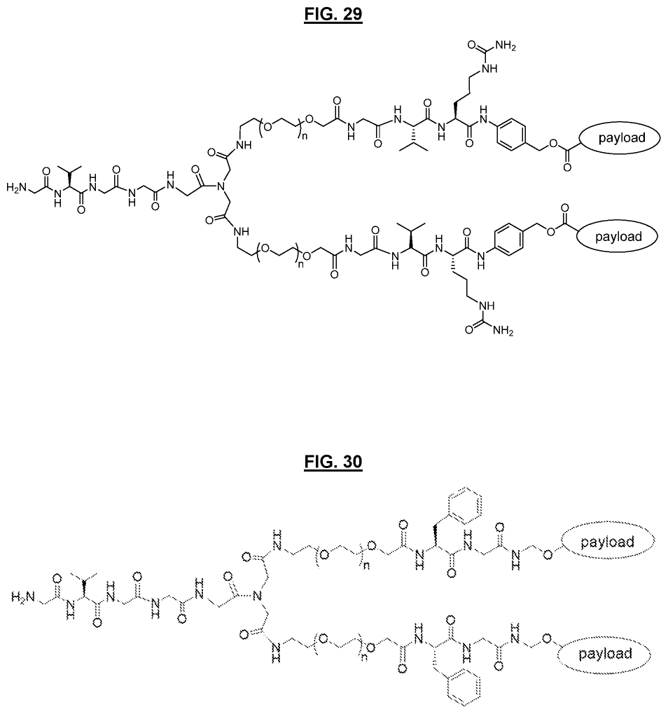

In addition, Z_{EGFR}-Fc-NQL was also ligated with Gly-Val-Ala-PABC-MMAE (FIG. 23). MMAE or monomethyl auristatin E is a potent antimitotic agent. Val-Ala-para-aminobenzylcarbamate (ValAla-PABC) is a linker that is cleavable by intracellular proteases and the Gly-Val dipeptide is a good acyl acceptor nucleophile substrate for PAL enzymes. Ligation of Z_{EGFR}-Fc-NQL (200μM) and Gly-Val-Ala-PABC-MMAE (800 μM) was catalyzed by VyPAL2 (0.002 eq) at pH 7 and 23° C. Again, the absence and presence of QC (0.0002 eq) gave a drastic difference in ligation efficiency (FIG. 6). In the presence of QC, the ligated product was obtained in ca. 80% yield after 4 h, whereas in the absence of it, the yield was only 42%. Because Z_{EGFR}-Fc-NQL is a dimer, the stoichiometric equivalence of the MMAE-containing nucleophile substrate was 2. The conjugate of the Z_{EGFR}-Fc fusion protein with MMAE is akin to an antibody-drug conjugate or ADC. This result firmly proves that the present PAL-QC coupled method can be used to prepare ADCs for the treatment of cancer and other diseases, and there is no need to use a large access of the acyl acceptor substrate in the ligation reaction. It is anticipated that this method will allow efficient ligation of monoclonal antibodies and other proteins with a broad range of linker-payload drug compounds as the acyl acceptor substrates (FIGS. 24-30).

### Example 4: Demonstration of the PAL-QC Cascade Scheme in Model Peptide Ligation Reaction using Mouse QC

The peptide ligation reaction between Ac-SYRNQL and GIGGIR was also tested using mouse QC and OaAEP1b-C247A.

Reaction conditions: 5 mM acyl acceptor and 5 mM acyl donor, OaAEP1b-C247A (0.0005 eq or 0.05 mol %) in 20 mM PBS (pH 7) at 37° C., with QC (0.00005 eq). In the absence of QC, the reaction gave the ligation product in about 45% yield (see FIG. 1). The yields were determined by HPLC (UV absorbance at 220 nm).

The results show that, just like human QC, mouse QC has the same effects in overcoming the reversibility seen in PAL-only ligations and by increasing the yield of a PAL-mediated ligation reaction.

### Summary

As the most powerful transpeptidases known to date, PALs have previously been shown to catalyze peptide and protein cyclization reactions very efficiently (Xia, Y., et al.; Angew. Chem. Int. Ed. 60, 22207-22211 (2021); Zhang, D., et al.; J. Am. Chem. Soc. 143, 8704-8712 (2021)), with a k_{cat}/Kₘ that is at least one order of magnitude higher than that of intermolecular ligation reactions. This is attributed to the entropically favorable nature of the intramolecular reaction. Moreover, the rigid conformation of the cyclized products often makes them resistant to PALs. Therefore, despite being also a transpeptidation reaction, PAL-catalyzed cyclization is usually irreversible. This is not the case for the bimolecular ligation reactions. Their reversibility generally limits the product yields to ≤50% at a 1:1 ratio between the two reaction partners. We show that this problem can be overcome by using a P1′ Gln in the acyl donor substrates since its a-amine can be quenched by lactamization upon cleavage of the Asn-Gln peptide bond. Pyroglutamyl formation can occur spontaneously, but it is a slow process. The reported rate constant of spontaneous pGlu formation of an N-terminal Gln is 1.7×10⁻⁶ s⁻¹ at pH 6, which corresponds to a half-life of about 4.7 days (Seifert, F., et al., Biochemistry 48, 11831-11833 (2009)). Human QC-catalyzed pGlu formation has a k_{cat} of 30 s⁻¹, representing a rate enhancement by seven orders of magnitude (Seifert, F., et al., Biochemistry 48, 11831-11833 (2009). The high efficiency of QC makes it ideally suited for coupled use with PALs. In our cascade enzymatic scheme, QC was used at one-tenth equivalence to the PAL enzyme which was used at 1:1000 or 1:2000 molar ratio to the substrate. Using this scheme, the yield of intermolecular ligations was greatly improved at equal or moderately higher molar equivalence of the acyl acceptor substrate to the acyl donor substrate. Our method is generally applicable with all PALs and to substrates of various sizes ranging from small peptides to large recombinant proteins. Compared to existing methods which utilize metal ions, synthetic chemicals or unnatural elements in the substrates to address the reversibility problem, this method uses an innocuous enzyme. Although the use of another enzyme may lead to cost related issues, this is not a big concern as QC can be easily expressed in E. coli and yeast systems. The high reaction yields and need for very low quantities of the enzymes also facilitate product purification and make the process cost-effective. Overall, this robust cascade enzymatic scheme according to the invention greatly increases the applicability of PAL-mediated ligation in the precision manufacturing of large protein conjugates.

### REFERENCES

Bi, X.; Yin, J.; Nguyen, G. K. T.; Rao, C.; Halim, N. B. A.; Hemu, X.; Tam, J. P.; Liu, C.-F. Enzymatic Engineering of Live Bacterial Cell Surfaces Using Butelase 1. Angew. Chem. Int. Ed. Engl. 2017, 56, 7822-7825.
Bi, X.; Yin, J.; Hemu, X.; Rao, C.; Tam, J. P.; Liu, C.-F. Immobilization and Intracellular Delivery of Circular Proteins by Modifying a Genetically Incorporated Unnatural Amino Acid. Bioconjugate Chem. 2018, 29, 2170-2175.
Bi, X.; Yin, J.; Zhang, D.; Zhang, X.; Balamkundu, S.; Lescar, J.; Dedon, P. C.; Tam, J. P.; Liu, C.-F. Tagging Transferrin Receptor with a Disulfide FRET Probe to Gauge the Redox State in Endosomal Compartments. Anal. Chem. 2020, 92, 12460-12466.
Busby, Jr., W. H.; Quackenbush, G.E.; Humm, J.; Youngblood, W. W.; Kizer, J. S. An Enzyme(s) That Converts Glutaminyl-peptides into Pyroglutamyl-peptides. Presence in Pituitary, Brain, Adrenal Medulla, and Lymphocytes. J. Biol. Chem. 1987, 262, 8532-8536.
Cao, Y.; Nguyen, G. K. T.; Tam, J. P.; Liu, C.-F. Butelase-mediated Synthesis of Protein Thioesters and Its Application for Tandem Chemoenzymatic Ligation. Chem. Commun. 2015, 51, 17289-17292.
Cao, Y.; Nguyen, G. K. T.; Chuah, S.; Tam, J. P.; Liu, C-F. Butelase-Mediated Ligation as an Efficient Bioconjugation Method for the Synthesis of Peptide Dendrimers. Bioconj. Chem. 2016, 27, 2592-2596.
Chen, H.; Lin, Z.; Arnst, K.; Miller, D.; Li, W. Tubulin Inhibitor-Based Antibody-Drug Conjugates for Cancer Therapy. Molecules 2017, 22, 1281
Doronina, S. O.; Mendelsohn, B. A.; Bovee, T. D.; Cerveny, C. G.; Alley, S. C.; Meyer, D. L.; Oflazoglu, E.; Toki, B. E.; Sanderson, R. J.; Zabinski, R. F. Enhanced Activity of Monomethylauristatin F through Monoclonal Antibody Delivery: Effects of Linker Technology on Efficacy and Toxicity. Bioconjugate Chem. 2006, 17, 114-124.
Harris, K. S.; Durek, T.; Kaas, Q.; Poth, A. G.; Gilding, E. K.; Conlan, B. F.; Saska, I.; Daly, N. L.; Van der Weerden, N. L.; Craik. D. J.; Anderson. M. A. Efficient Backbone Cyclization of Linear Peptides by a Recombinant Asparaginyl Endopeptidase. Nat. Commun. 2015, 6, 10199.
Hemu, X.; Sahili, A. E.; Hu, S.; Wong, K.; Chen, Y. Wong, Y. H.; Zhang, X.; Serra, A.; Goh, B. C.; Darwis, D. A.; Chen, M. W.; Sze, S. K.; Liu, C-F.; Lescar, J.; Tam, J. P. Structural Determinants for Peptide Bond Formation by Asparaginyl Ligases. Proc. Natl. Acad. Sci. 2019, 116, 11737-11746.
Hoyt, E. A.; Cal, P. M. S. D.; Oliverira, B. L.; Bernardes, G. L. Contemporary Approaches to Site-selective Protein Modification. Nat. Rev. Chem. 2019, 3, 147-171.
Jost, C.; Schilling, J.; Tamaskovic, R.; Schwill, M.; Honegger, A.; and Pluckthun, A. Structural Basis for Eliciting a Cytotoxic Effect in HER2-overexpressing Cancer Cells via Binding to the Extracellular Domain of HER2. Structure 2013, 21, 1979-1991.
Lotze, J.; Reinhardt, U.; Seitz, O.; Beck-Sickinger, A. G. Peptide-tags for Site-specific Protein Labelling in vitro and in vivo. Mol Biosyst. 2016, 12, 1731-1745.
Nguyen, G. K. T.; Wang, S.; Qiu, Y.; Hemu, X.; lian, Y.; Tam, J. P. Butelase 1 Is an Asx-specific Ligase Enabling Peptide Macrocyclization and Synthesis. Nat Chem Biol. 2014, 10, 732-738.
Nguyen, G. K. T.; Qiu, Y.; Cao, Y.; Hemu, X.; Liu, C.-F.; Tam, J. P. Butelase-mediated Cyclization and Ligation of Peptides and Proteins. Nat. Protoc. 2016, 11, 1977-1988.
Nguyen, G. K. T.; Kam, A.; Loo, S.; Jansson, A. E.; Pan, L. X.; Tam, J. P. Butelase 1: A Versatile Ligase for Peptide and Protein Macrocyclization. J. Am. Chem. Soc. 2015, 137, 15398-15401.
Nguyen, G. K. T.; Cao, Y.; Wang, W.; Liu, C-F.; and Tam, J. P. Site-Specific N-Terminal Labeling of Peptides and Proteins using Butelase 1 and Thiodepsipeptide. Angew. Chem. Int. Ed. 2015, 54, 15694-15698.
Pishesha, N.; Ingram, J. R.; Ploegh, H. L. Sortase A: A Model for Transpeptidation and Its Biological Applications. Annu. Rev. Cell Dev. Biol. 2018, 34, 163-88.
Rehm, F. B. H.; Tyler, T. J.; Xie, J.; Yap, K.; Durek, T.; Craik, D. J. Asparaginyl Ligases: New Enzymes for the Protein Engineer's Toolbox. ChemBioChem 2021, 22, 2079-2086.
Rehm, F. B.H.; Harmand, T. J.; Yap, K.; Durek, T.; Craik, D. J.; Ploegh, H. L. Site-Specific Sequential Protein Labeling Catalyzed by a Single Recombinant Ligase. J. Am. Chem. Soc. 2019, 141 (43), 17388-17393.
Rehm, F. B. H.; Tyler, T. J.; Yap, K.; Durek, T.; Craik, D. J. Improved Asparaginyl-Ligase-Catalyzed Transpeptidation via Selective Nucleophile Quenching. Angew. Chem. Int. Ed. 2021, 60, 4004-4008.
Rosen, C. B.; Francis, M. B. Targeting the N Terminus for Site-selective Protein Modification. Nat. Chem. Biol. 2017, 13, 697-705.
Schilling, S.; Hoffmann, T.; Rosche, F.; Manhart, S.; Wasternack, C.; Demuth. H.-U. Heterologous Expression and Characterization of Human Glutaminyl Cyclase: Evidence for a Disulfide Bond with Importance for Catalytic Activity. Biochemistry 2002, 41, 10849-10857.
Seifert, F.; Schulz, K.; Koch, B.; Manhart, S.; Demuth H.-U.; Schilling, S. Glutaminyl Cyclases Display Significant Catalytic Proficiency for Glutamyl Substrates. Biochemistry 2009, 48, 11831-11833.
Sgouros, G.; Bodei, L.; McDeitt, M. R.; Nedrow, J. R. Radiopharmaceutical Therapy in Cancer: Clinical Advances and Challenges. Nat. Rev. Drug Dis. 2020, 19, 589-608.
Sletten, E. M.; Bertozzi, C. R. Bioorthogonal Chemistry: Fishing for Selectivity in a Sea of Functionality. Angew. Chem. Int. Ed. 2009, 48, 6974-6998.
Spicer, C. D.; Davis, B. G. Selective Chemical Protein Modification. Nat. Commun. 2014, 5, 4740.
Ståhl, S.; Gräslund, T.; Karlström, A. E.; Frejd, F. Y.; Nygren, P. Å.; Löfblom, J. Affibody Molecules in Biotechnological and Medical Applications. Trends Biotechnol. 2017, 35, 691-712.
Steiner, D.; Forrer, P.; Pluckthun, A. Efficient Selection of DARPins with Sub-nanomolar Affinities using SRP Phage Display. J. Mol. Biol. 2008, 382, 1211-1227.
Tam, J. P.; Chan, N.; Liew, H. T.; Tan, S. J.; Chen, Y. Peptide Asparaginyl Ligases—Renegade Peptide Bond Makers. Science China Chemistry 2020, 63, 296-307.
Tang, T. M. S.; Cardella, D.; Lander, A. J.; Li, X.; Escudero, J. S.; Tsai, Y-H.; Luk, L. Y. P. Use of an Asparaginyl Endopeptidase for Chemo-enzymatic Peptide and Protein Labeling. Chem. Sci. 2020, 11, 5881-5888.
Viola-Villegas, N.; Doyle, R. P. The Coordination Chemistry of 1,4,7,10-Tetraazacyclododecane-N,N′, N″, N′″-tetraacetic Acid (H₄DOTA): Structural Overview and Analyses on Structure—Stability Relationships. Coordination Chemistry Reviews 2009, 253, 1906-1925.
Weeks, A. M.; Wells, J. A. Subtiligase-Catalyzed Peptide Ligation. Chem. Rev. 2020, 120, 3127-3160.
Xia, Y.; To, J.; Chan, N.-Y.; Hu, S.; Liew, H. T.; Balamkundu, S.; Zhang, X.; Lescar, L.; Bhattacharjya, S.; Tam, J. P.; Liu, C.-F. N^{Y}-Hydroxyasparagine: A Multifunctional Unnatural Amino Acid That is a Good P1 Substrate of Asparaginyl Peptide Ligases. Angew. Chem. Int. Ed. 2021, 60, 22207-22211.
Yang, R.; Wong, Y. H.; Nguyen, G. K. T.; Tam, J. P.; Lescar, L.; Wu, B. Engineering a Catalytically Efficient Recombinant Protein Ligase. J. Am. Chem. Soc. 2017, 139, 5351-5358.
Zhang, D.; Wang, Z.; Hu, S.; Balamkundu, S.; To, J.; Zhang, X.; Lescar, J.; Tam, J. P.; Liu, C.-F. pH-Controlled Protein Orthogonal Ligation Using Asparaginyl Peptide Ligases. J. Am. Chem. Soc. 2021, 143, 8704-8712.
Zhang, G.; Zheng, S.; Liu, H.; Chen. P. R. Illuminating Biological Processes through Site-specific Protein Labeling. Chem. Soc. Rev. 2015, 44, 3405-3717.
Zhang, Y.; Park, K. Y.; Suazo, K. F.; Distefano, M. D. Recent Progress in Enzymatic Protein Labelling Techniques and Their Applications. Chem. Soc. Rev. 2018, 47, 9106-9136.

1. A method of enzymatic peptide ligation, said method comprising providing
i) a peptidyl asparaginyl ligase (PAL) and a glutaminyl cyclase (QC);
ii) a first peptide or protein having a P1-P1′-P2′ tripeptide PAL motif as an acyl donor, wherein P1 is Asn or Asp, P1′ is Gln or Glu and P2′ is a hydrophobic amino acid or a β-branched amino acid;
iii) a second peptide or protein which may be the same or different to the first peptide or protein, having a P1″-P2″ motif as an acyl acceptor at the N-terminus, wherein P1″ is any amino acid and P2″ is a hydrophobic amino acid or a β-branched amino acid;
iv) contacting the peptidyl asparaginyl ligase (PAL) and the glutaminyl cyclase (QC) with said first and second peptides or proteins;
wherein PAL cleaves the first peptide or protein after P1 in the tripeptide PAL motif and ligates said first peptide or protein to the P1″-P2″ motif of said second peptide or protein, and QC cyclizes P1′ in the released P1′-P2′ dipeptide motif to pyroglutamyl (pGlu).

2-16. (canceled)

17. A conjugate comprising a first peptide or protein and a second peptide or protein which may be the same or different to the first peptide or protein, the second peptide or protein having a N-terminus, wherein one of said first and second peptides or proteins is an epitope-binding peptide or protein, and the other peptide or protein comprises a payload, and wherein the first peptide or protein is ligated to the N-terminus of the second peptide or protein via-P1-P1″-P2″-, wherein:
P1 is Asn or Asp;
P1″ is any amino acid;
P2″ is a hydrophobic amino acid or β-branched amino acid.

18. The conjugate according to claim 17, wherein the C-terminus of the first peptide or protein is ligated to the N-terminus of the second peptide or protein.

19. The conjugate according to claim 17, wherein the epitope-binding peptide or protein is an antibody or functional fragment thereof.

20. The conjugate according to claim 19, wherein the antibody or functional fragment thereof is selected from minibody, diabody, scFv, nanobody and F(ab′)2.

21. The conjugate according to claim 17, wherein P2″ is selected from: Leu, Phe, Tyr, Trp, Val, Ile and Thr.

22. The conjugate according to claim 21, wherein P2″ is Val or Ile.

23. The conjugate according to claim 17, wherein the payload further comprises a payload-releasing linkage.

24. The conjugate according to claim 17, wherein the payload is an imaging agent or a therapeutic agent.

25. The conjugate according to claim 24, wherein the imaging agent is a radiolabel chelator or an optical label.

26. The conjugate according to claim 25, wherein the radiolabel chelator is selected from 1,4,7-triazacyclononanetriacetic acid (NOTA), 1,4,7,10-tetraazacyclododecane-1,4,7,10-tetraacetic acid (DOTA) and 1,4,7-triazacyclononane-1-glutaric acid-4,7-diacetic acid (NODAGA).

27. The conjugate according to claim 24, wherein the therapeutic agent is selected from:
a) Monomethyl auristatin E (MMAE);
b) radiolabelled DOTA;
c) Exatecan;
d) Glycolyl-exatecan;
d) Maytansine;
e) PBD dimer;
f) Auristatin E;
g) SN-38; and
h) α-amanitin.

28. The conjugate according to claim 17, wherein the payload comprises a drug linked to the conjugate via an amine group in the drug, and the drug is selected from: 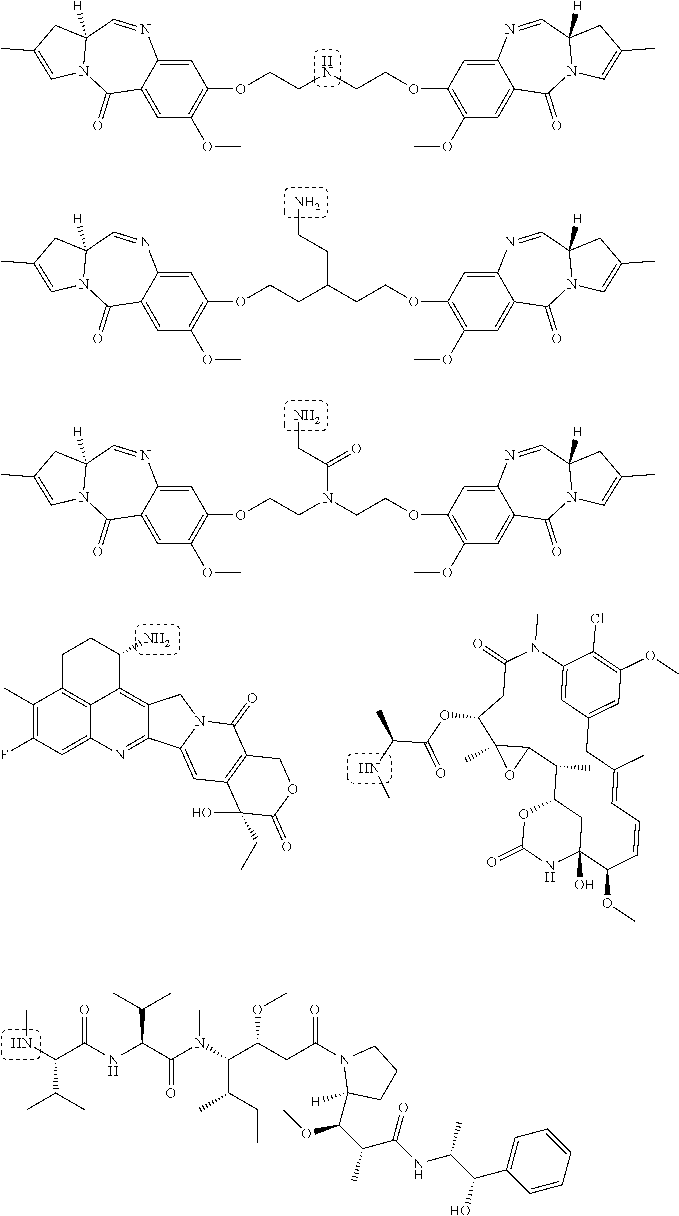
wherein [symbol] indicates the amine group, and the amine group can be modified with a linker.

29. The conjugate according to claim 17, wherein the peptide or protein comprising a payload is selected from:

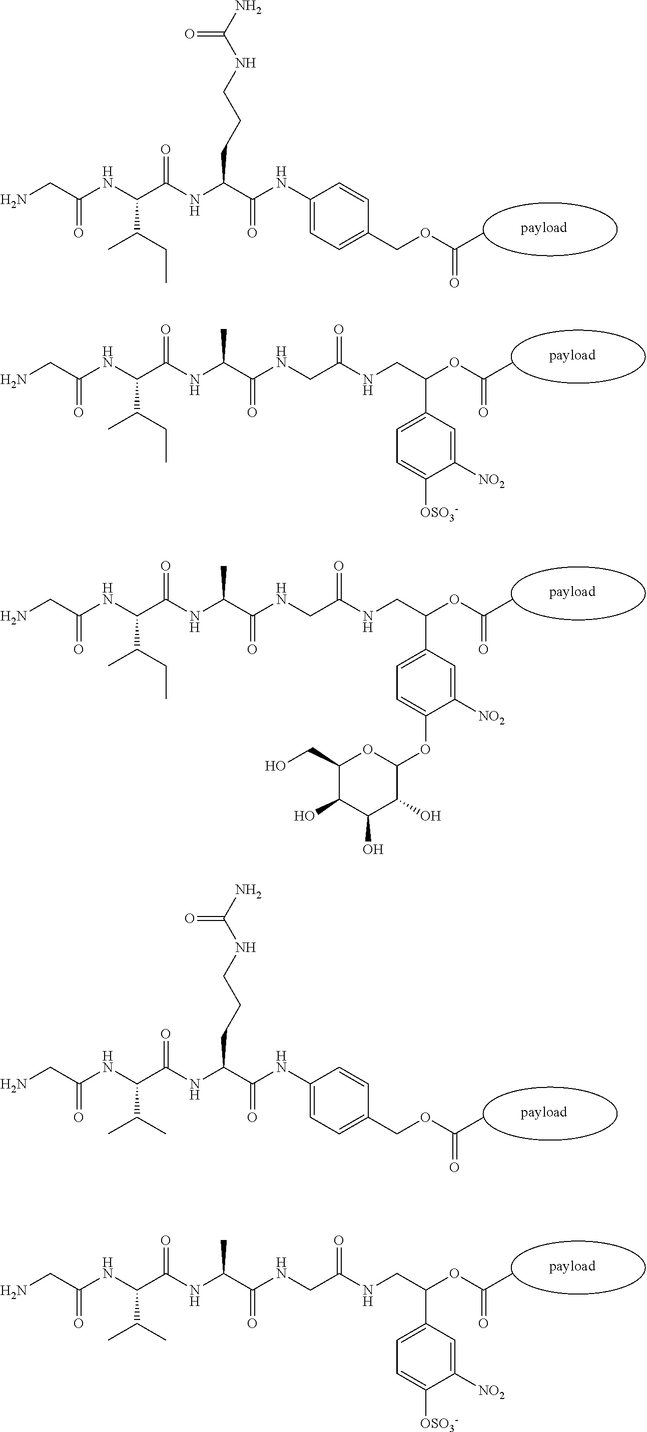

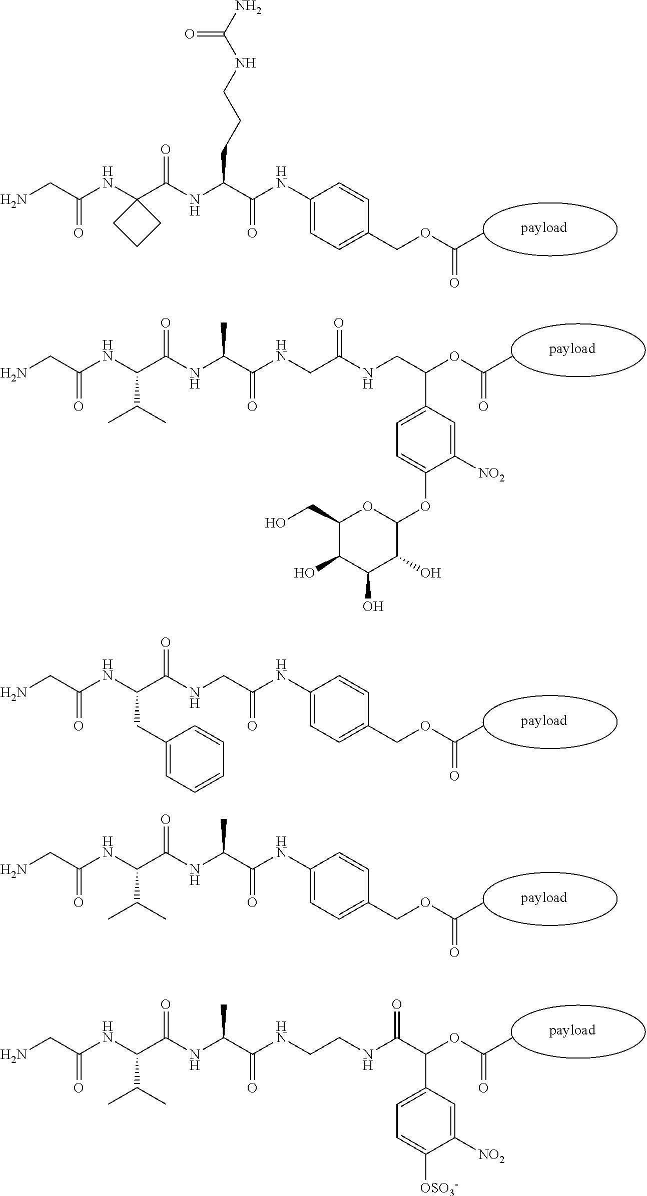

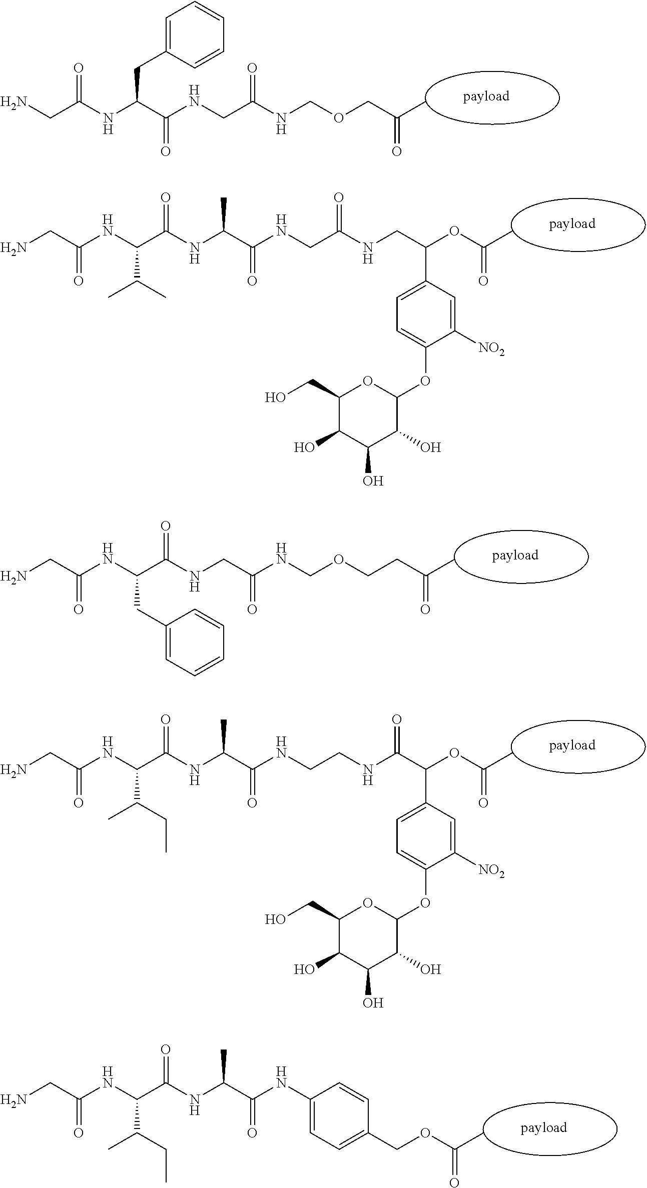

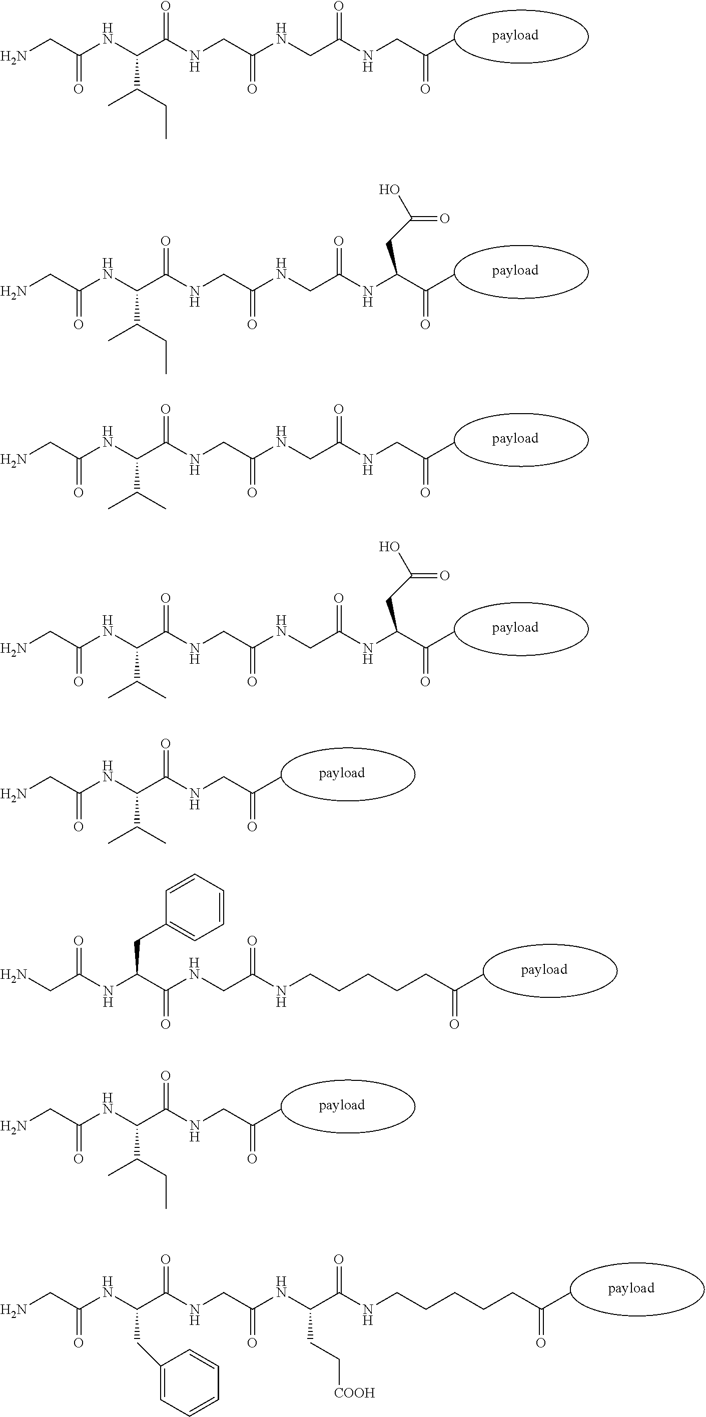

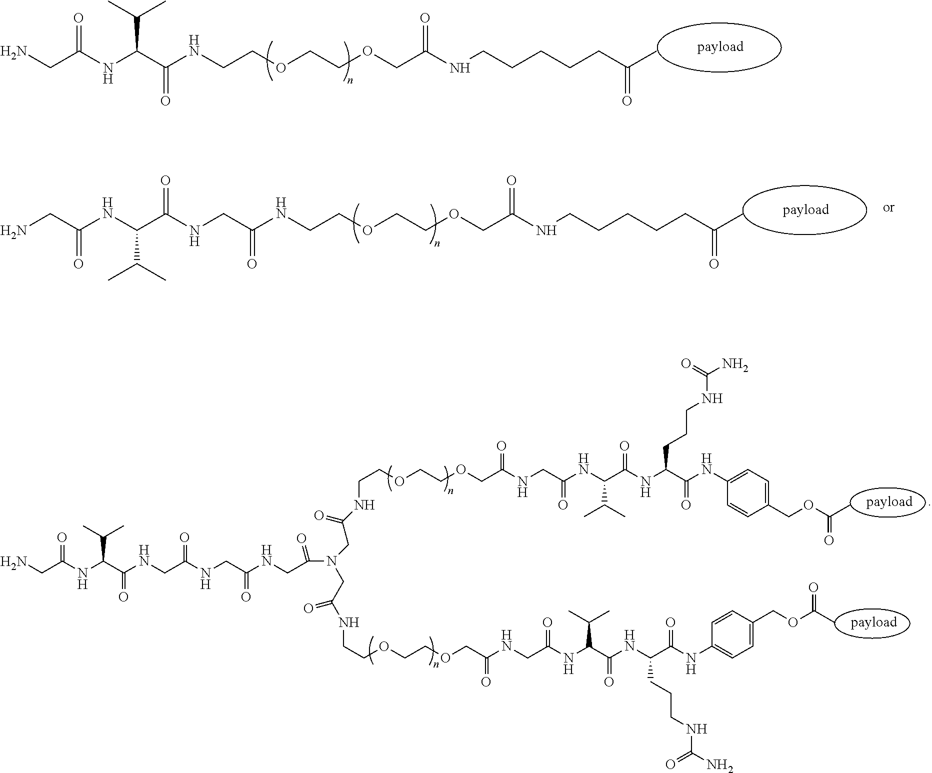

30. The conjugate according to claim 29, wherein [symbol] payload is a drug linked via an amine group in the drug, and the drug is selected from: 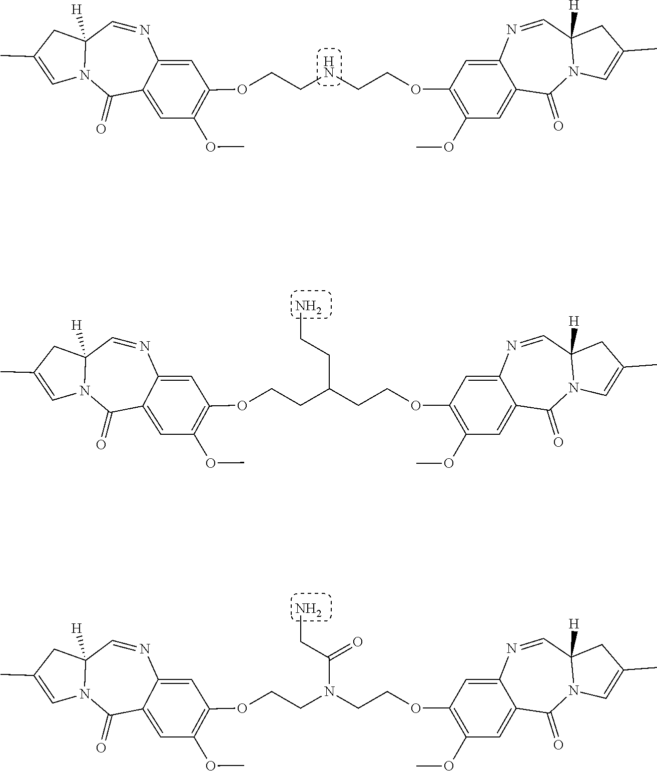 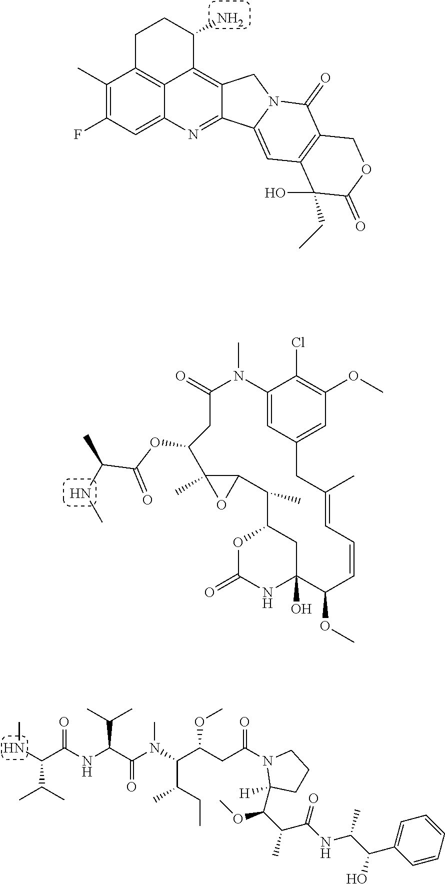
wherein [symbol] indicates the amine group.

31. The conjugate according to claim 17, wherein the payload comprises a drug linked to the conjugate via a hydroxy group in the drug, and the drug is selected from: 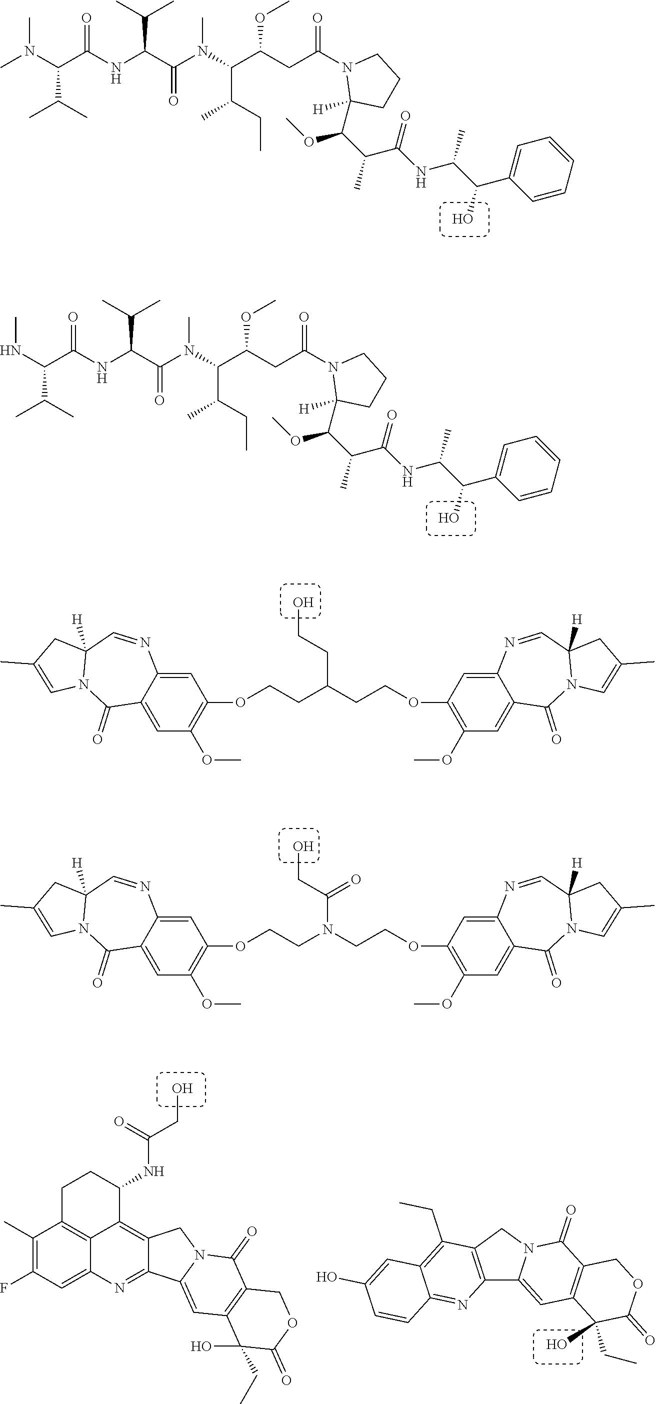 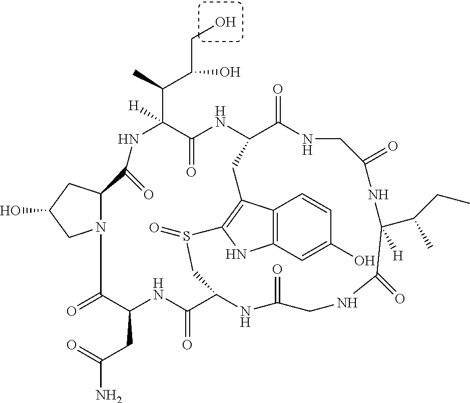 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]
wherein [symbol] indicates the hydroxy group, and the hydroxy group can be modified with a linker.

32. The conjugate according to claim 17, wherein the peptide or protein comprising a payload is selected from:

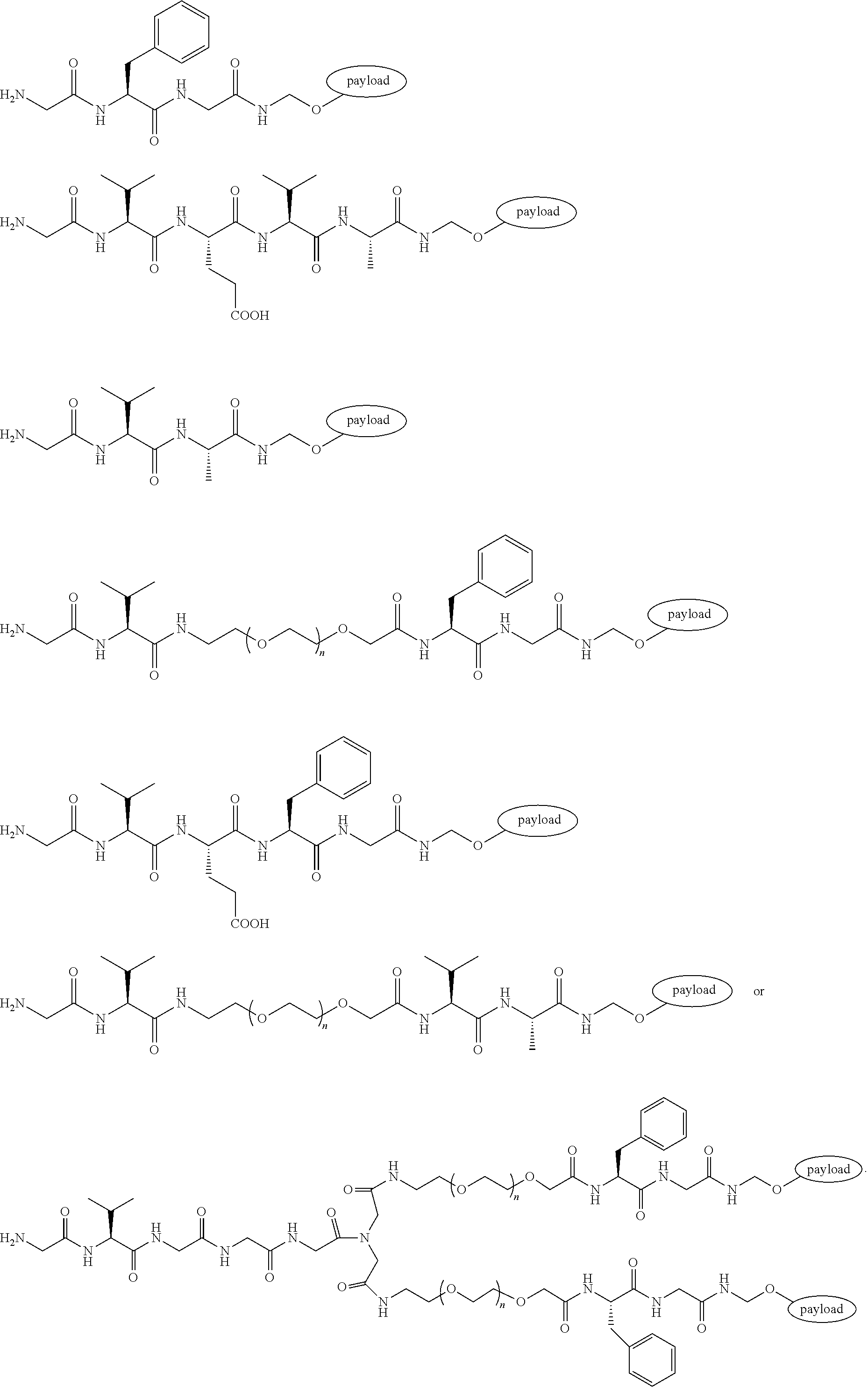

33. The conjugate according to claim 32, wherein [symbol] is a drug linked via a hydroxy group in the drug, and the drug is selected from: 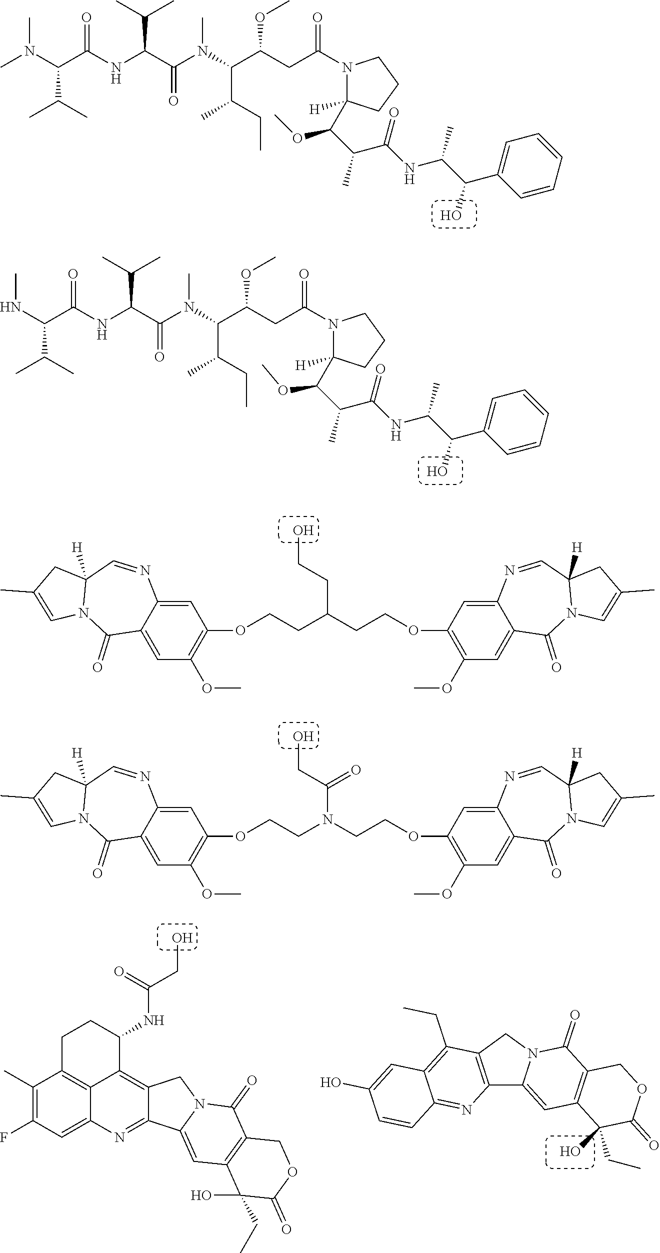 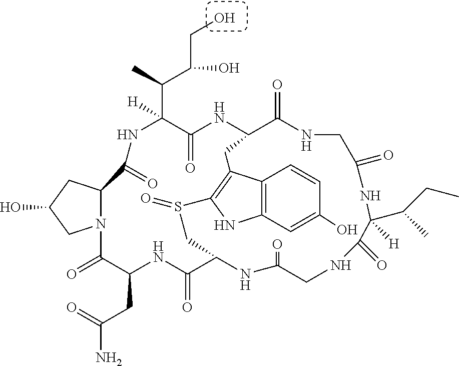 [SMILES: CC[C@H](C)[C@@H]1NC(=O)CNC(=O)[C@@H]2Cc3c([nH]c4cc(O)ccc34)S(=O)C[C@H](NC(=O)CNC1=O)C(=O)N[C@@H](CC(N)=O)C(=O)N1C[C@H](O)C[C@H]1C(=O)N[C@@H]([C@@H](C)[C@@H](O)CO)C(=O)N2]
wherein [symbol] indicates the hydroxy group.

34. The conjugate according to claim 17, wherein the peptide comprising a payload is selected from:

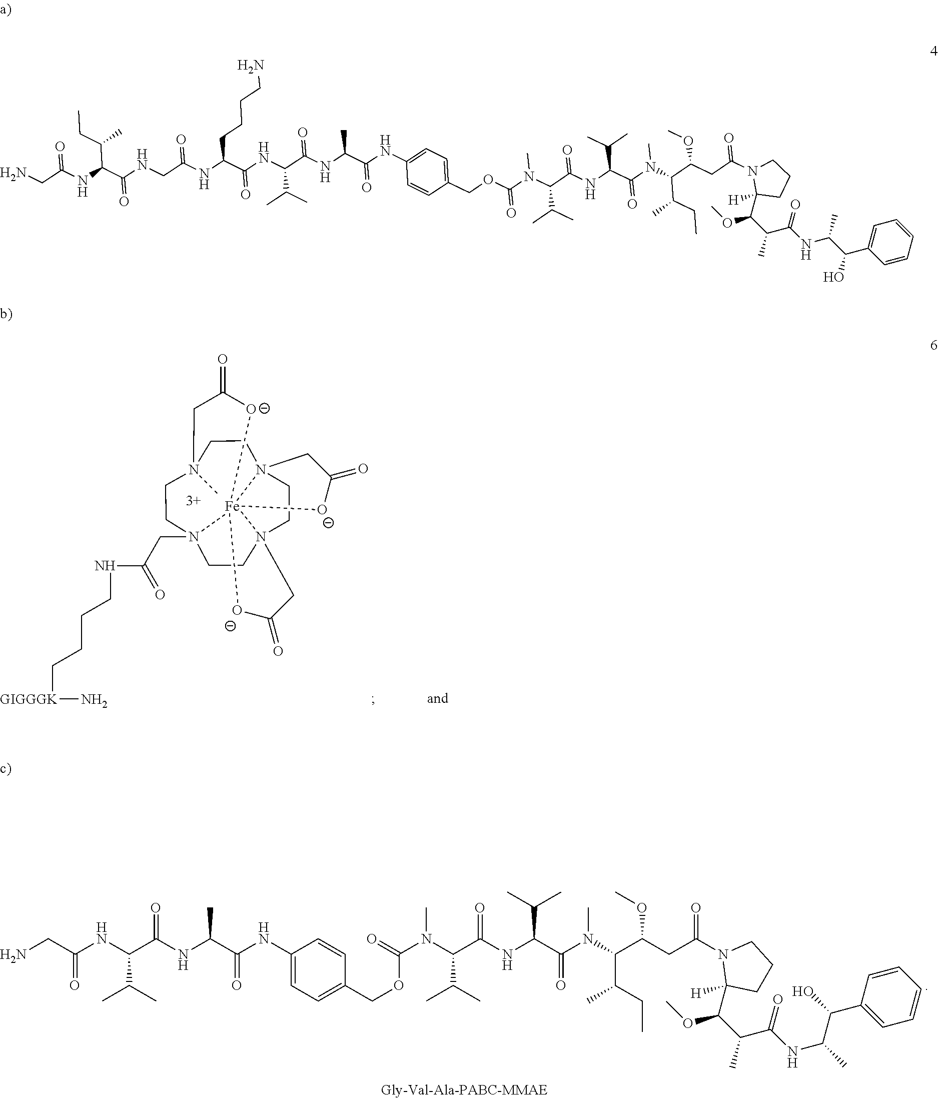

35. A conjugate resulting from a method of enzymatic peptide ligation, said method comprising providing:
i) a peptidyl asparaginyl ligase (PAL) and a glutaminyl cyclase (QC);
ii) a first peptide or protein having a P1-P1′-P2′ tripeptide PAL motif as an acyl donor, wherein P1 is Asn or Asp, P1 is Gln or Glu and P2 is a hydrophobic amino acid or a β-branched amino acid;
iii) a second peptide or protein which may be the same or different to the first peptide or protein, having a P1″-P2″ motif as an acyl acceptor at the N-terminus, wherein P1″ is any amino acid and P2″ is a hydrophobic amino acid or a β-branched amino acid;
iv) contacting the peptidyl asparaginyl ligase (PAL) and the glutaminyl cyclase (QC) with said first and second peptides or proteins;
wherein PAL cleaves the first peptide or protein after P1 in the tripeptide PAL motif and ligates said first peptide or protein to the P1″-P2″ motif of said second peptide or/protein, and QC cyclizes P1′ in the released P1′-P2′ dipeptide motif to pyroglutamyl (pGlu), and wherein one of said first and second peptides or proteins is an epitope-binding peptide or protein and the other peptide or protein comprises a payload.
